(19) United States
(12) Patent Application Publication
(10) Pub. No.: US 2025/0295693 A1
(43) Pub. Date: Sep. 25, 2025

(54) HYBRID TUMOR/CANCER THERAPY BASED ON TARGETING THE RESOLUTION OF OR INDUCING TRANSCRIPTION-REPLICATION CONFLICTS (TRCS)

(71) Applicant: Julius-Maximilians-Universität Würzburg, Würzburg (DE)

(72) Inventors: Martin EILERS, Würzburg (DE); Armin WIEGERING, Würzburg (DE); Gabriele BÜCHEL; Mareike MÜLLER, Würzburg (DE); Bastian KRENZ, Rimpar (DE); Hanna DEINLEIN, Würzburg (DE); Anneli GEBHARD-WOLF, Würzburg (DE)

(73) Assignee: Julius-Maximilians-Universität Würzburg, Würzburg (DE)

(21) Appl. No.: 18/682,248

(22) PCT Filed: Aug. 17, 2022
(86) PCT No.: PCT/EP2022/073008

§ 371 Date: Feb. 8, 2024

(30) Foreign Application Priority Data
Aug. 18, 2021 (EP) 21192020.2

(51) Int. Cl. A61K 35/17; A61K 40/11; A61K 40/31; A61K 40/42; C07K 14/55; C07K 14/705; C07K 14/725; C07K 16/28; C12N 5/0783

(52) U.S. Cl. CPC A61K 35/17; A61K 40/11; A61K 40/31; A61K 40/421; C07K 14/55; C07K 14/7051; C07K 14/70517; C07K 16/2827; C12N 5/0636; A61K 2239/13; A61K 2239/17; A61K 2239/21; C07K 2319/02; C12N 2510/00

(57) ABSTRACT

The present invention relates to a hybrid treatment of a tumor/cancer, said treatment comprises (i) targeting the resolution of transcription-replication conflicts in a tumor/cancer; or (ii) inducing in a tumor/cancer; and the use/administration of an immune cell, or a progenitor cell thereof, which is resistant against/less susceptible to said targeting/inducing. The immune cell, or progenitor cell thereof, is envisaged to target a/said (cell(s) of) a/said (solid) tumor/cancer. The present invention further relates to a respective immune cell, or a progenitor cell thereof. The present invention further relates to a pharmaceutical composition comprising the immune cell and/or a progenitor cell thereof and to a pharmaceutical composition, a kit or a combination (e.g. set of two/three components) comprising the immune cell and/or a progenitor cell thereof and a DDIA. The present invention further relates to methods of screening for a target of a DDIA which is resistant against/less susceptible to said DDIA or for an agent that is capable of inhibiting a target in a cell of a cancer/tumor and thereby inducing DNA damage and/or preventing resolution of DNA damage in said cell of a cancer/tumor; and that is incapable of inhibiting said target which is resistant against/less susceptible to said agent in an immune cell, or progenitor cell thereof, and thereby not inducing DNA damage and/or not preventing resolution of DNA damage in said immune cell or progenitor cell thereof.

This application is the National Phase application of International Application No. PCT/EP2022/073008 filed Aug. 17, 2022, which is incorporated herein by reference in its entirely. International Application No. PCT/EP2022/073008 claims priority to European Patent Application No. 21192020.2 filed Aug. 18, 2021.

The present invention relates to a hybrid treatment of a tumor/cancer, said treatment comprises (i) targeting the tumor/cancer by a chemotherapeutic (e.g. by a DNA damage-inducing agent (DDIA)), in particular targeting the resolution of transcription-replication conflicts (TRCs) in cells of said tumor/cancer; or (ii) inducing (by a DDIA) TRCs in cells of said tumor/cancer. The hybrid treatment according to the invention further comprises the use/administration of an immune cell, or a progenitor cell thereof, which is resistant against said targeting/inducing (e.g. by the DDIA) or which exhibits reduced susceptibility to said targeting/inducing (e.g. by the DDIA). The immune cell, or progenitor cell thereof, is envisaged to target a/said cells of said tumor/cancer. The present invention further relates to an immune cell, or a progenitor cell thereof, which is resistant against a DDIA or which exhibits reduced susceptibility to a DDIA. The present invention further relates to a pharmaceutical composition comprising the immune cell and/or a progenitor cell thereof according to the invention. The present invention further relates to a pharmaceutical composition, a kit or a combination (e.g. set of two or three components) comprising (i) an immune cell and/or a progenitor cell thereof according to the invention and (ii) a DDIA. The present invention further relates to methods of screening for (a mutant of) a target of a which is resistant against said DDIA or has reduced susceptibility to said DDIA or for an agent that is (i) capable of inhibiting a target in a cell of a cancer/tumor and thereby inducing DNA damage and/or preventing resolution of DNA damage in said cell of a cancer/tumor; and that is (ii) incapable of inhibiting (a mutant of) said target which is resistant against said agent or has reduced susceptibility to said agent in an immune cell, or progenitor cell thereof, and thereby not inducing DNA damage and/or not preventing resolution of DNA damage in said immune cell or progenitor cell thereof.

In all organisms, ranging from prokaryotes to humans, deregulated transcription raises the inherent risk of conflicts with the replication fork (Garcia-Muse, Nature Reviews Molecular Cell Biology 17, 2016, 553-563; Hamperl, Cell 167, 2016, 1455-1467). One reason for this is that perturbances in transcription lead to the accumulation of R-loops, which are stable hybrids between nascent mRNA and the double-stranded DNA (Crossley, Mol Cell 73, 2019, 398-411).

R-loop formation displaces one DNA strand, causing frequent single-strand DNA breaks, and are an impediment to a replication fork, causing collisions between RNA polymerases and the replication fork. If collisions occur, double-strand breaks are caused due to the accumulation of excessive torsional stress between the DNA and RNA polymerase complexes. Such conflicts are termed “transcription-replication conflicts” (TRCs) (e.g. Garcia-Muse, loc.cit.). TRCs are particularly difficult to resolve when RNA polymerase stalls, for example due to low nucleotide concentrations (Noe Gonzalez, Nature reviews 22, 2021, 3-21).

A hallmark of virtually all tumors is the presence of mutations that directly or indirectly deregulate RNA transcription, affecting either large groups of genes (such as cell cycle- or growth-promoting genes) and/or global transcription rates. As mentioned, this generates a problem during DNA replication, since transcription and replication act on the same DNA template and the respective enzyme complexes can collide (the mentioned issue of TCRs).

Emerging evidence shows that TCRs are prevalent in tumor cells that grow rapidly in metabolically challenging conditions. Thus, it is likely that mechanisms that resolve such conflicts are critical for the survival of tumor cells and that successfully targeting these mechanisms will open a wide therapeutic window.

Central mechanisms have been identified that enable tumor cells to escape conflicts of the replication fork with RNA polymerases I and II (Hamperl, Cell 167, 2016, 1455-67). It was also shown that targeting these mechanism causes significant DNA damage and allows the eradication of tumors in animal model systems, for example in the pediatric tumor entity, neuroblastoma (Brockmann, Cancer cell 24, 2013, 75-89; Roeschert, Nature Cancer, 2021, https://doi.org/10.1038/s43018-020-00171-8)).

In proteomic analyses, it was observed that MYCN associates with an unexpected set of cellular proteins and that this association is highly dynamic during the cell cycle. Specifically, it was found that the Aurora-A kinase, which associates with MYCN (Brockmann loc.cit.; Otto, Cancer cell 15, 2009, 67-78), competes with several other co-factors and causes a switch in MYCN complexes during the S-phase of the cell cycle. Disruption of this exchange causes replication stress as witnessed by activation of the ATR kinase, which senses stalling of replication forks (Buchel, Cell reports 21, 2017, 3483-97). Subsequent analyses showed that the MYCN protein enables tumor cells to resolve TRCs. By now, two molecular pathways by which MYCN regulates these conflicts have been identified (Roeschert loc.cit.). First, the BRCA1 protein can be recruited to promoters. The major trigger for this is the accumulation of R-loops, which are stable hybrids between the nascent mRNA and one strand of the DNA. R-loops are major obstacles for the advancing replication fork. Recruitment of BRCA1 in turn leads to the recruitment of the mRNA decapping enzyme, DCP1A, which then terminates transcription to resolve R-loop formation (Herold, Nature 567, 2019, 545-9). Second, Aurora-A phosphorylates histone H3.3 at serine 10 ahead of the replication fork. This promotes the formation of heterochromatin, presumably because it blocks histone acetylation by TFIIIC. Aurora-A dependent formation of stable heterochromatin antagonizes R-loop formation (R-loops are free of nucleosomes) during S-phase and is required for S-phase progression (Roeschert loc.cit.).

Further, it was found that triggering replication conflicts by inhibition of the Aurora-A kinase renders tumor cells dependent on the ATR kinase for survival since ATR stabilizes and maintains stalling replication forks. Combined inhibition of both kinases induces rampant and highly tumor-specific DNA damage, with almost all tumor cells undergoing apoptosis. This correlates with an often complete tumor regression, when mice are treated with a combination of low and non-toxic concentrations inhibitors of both kinases. Most importantly, the combined treatment greatly extends survival, often far beyond the end of treatment. Indeed, a subset of animals are cured. Additionally, not only a tumor-intrinsic effect was observed, but also an infiltration and activation of the immune system (Roeschert loc.cit.). However, there are two (human) Aurora-A alleles, T217D and T217E, which have been demonstrated to be resistant against available Aurora-A inhibitors (Sloane, ACS Chem Biol 5, 2010, 563-576).

Further, the induction of DNA damage in tumor cells not only impairs the genome stability of tumor cells, but also sensitizes tumors to immune cell-mediated killing. Specific signaling pathways such as the STING pathway recognize damaged DNA, aberrant RNA species and replication intermediates, induce the synthesis of cytokines and promote antigen presentation (Hopfner, Nature reviews 21, 2020, 501-21). This can be exploited to enhance anti-tumoral cellular immune therapies, since the induction of immunogenic DNA damage enhances cytokine-dependent recruitment and subsequent killing by CAR T cells (Srivastava, Cancer cell 39, 2021, 193-208 e110).

Despite progress in understanding of the molecular factors that initiate and maintain tumor growth, and despite the resulting advances in tumor therapies, however, many tumors, for example solid tumors like pancreatic ductal adenocarcinoma (PDAC) and metastatic colorectal carcinoma (CRC), still present large and unmet clinical needs, and patients continue to have a poor prognosis. One reason for this situation is that such tumors (e.g. PDAC and CRC) are driven by largely “undruggable” mutations, such as mutations in the KRAS oncogene (pancreas) (Borazanci, Clin Cancer Res 23, 2017, 1629-37) and the WNT signaling pathways (colon) (Fearon, Annu Rev Pathol 6, 2011, 479-507). In addition, immune therapeutic approaches have not achieved clinical success in most solid tumors except for a subset of CRC patients with mismatch repair-deficient mutations or microsatellite instability. Further, such tumor entities (e.g. of PDAC and CRC) almost universally harbor mutations that deregulate transcription. For example, in PDAC, the KRAS mutations in conjunction with loss of the CDKN2A tumor suppressor gene (encoding the p16Ink4A cell cycle inhibitor) deregulate E2F-dependent transcription leading to the constitutive expression of cell cycle-regulatory genes. In addition, the loss of the SMAD and p53 tumor suppressor genes deregulate MYC expression, leading to enhanced transcription of growth promoting genes. For example, in CRC, mutations in the WNT signaling pathway deregulate transcription by both RNA Polymerase I (leading to high level of ribosomal RNA synthesis) and RNA polymerase II via the stabilization of a critical transcription co-activator protein, beta-catenin. One main reason that nearly all solid tumors are resistant to current immunotherapy approaches is that the proto-oncogene MYC, which is deregulated in the majority of solid tumors, drives a number of immune evasion mechanisms. One of the critical mechanisms is MYC-driven secretion of lactate into the tumor environment. Therapeutic approaches that lead to the reduction of MYC and thus reduction of immune evasion, however, also inhibit the expression of MYC in immune cells (e.g. T-cells) that could eradicate the tumor. Since MYC is also required for immune cell expansion, these therapies have little or no therapeutic window.

Further, the survival of patients with, for example, metastasized colon tumors is mainly limited by the growth of metastases in the liver. While single metastasis can be cured by simple resection and have a good prognosis, this drops with bilobar metastases. For these metastatic tumors a two-step surgical procedure has been proposed to prevent postoperative liver failure (Lang, Cancer cell 7, 2007, 469-83). In the first step a small part of the liver is cleaned from metastases and portal blood flow to the larger, non-cleaned lobe is cut. While this “cured” section regenerates, the other lobe partially contributes to sustain sufficient liver function. When the “cured” lobe reaches a functionally sufficient volume the still metastases carrying part can be removed, but tumor progression in the tumor-bearing lobe can cause unresectability. Thus there is still the need of a molecular strategy that can be combined with this surgical strategy and that suppresses the growth of colon metastases while allowing liver regeneration. Such a molecular strategy may have the potential to cure a significant fraction of poor-prognosis patients, like those which suffer from bilobar metastases.

Further, although current chemotherapeutic regimes and DDIAs (cf., for example, Wang, J. Biol. Chem. 274(31), 1999, 22060-4), respectively, may be aided by immune therapies, they do not only inhibit the growth of tumor/cancer cells, but also other (highly) proliferating cells like immune cells (or their progenitors). In other words, current chemotherapeutic regimes, and DDIAs, respectively, do not induce DNA damage in a tumor/cancer cell type-specific manner. Thus, there is the drawback that therapeutic effects of, for example, immune cell engagement are also limited by chemotherapeutic drugs and DDIAs, respectively. At the same time, as mentioned, the efficacy of current cellular immune therapies is still limited, in particular in most solid tumors.

There is thus an unmet need in the field of (solid) tumor/cancer treatment to inflict damage in cancers/tumors (e.g. DNA damage in cancer/tumor cells) more effectively and/or in a tumor/cancer cell type-specific manner, i.e. only in the tumor/cancer cells, but not (or at least in a lower degree) in other cells, in particular not in other (highly) proliferating cells like immune cells (or their progenitors), which may be used concomitantly in immunotherapy of (solid) tumors/cancers.

The problem underlying the present invention is therefore the provision of means and methods for an improved medical intervention of (solid) tumors/cancers, in particular in the context of immune cells-aided chemotherapies of (solid) tumors/cancers, more particular DDIA-based and immune cells-aided chemotherapies of (solid) tumors/cancers, especially of those with large and currently unmet clinical needs (“undruggable” (solid) tumors/cancers).

The technical problem is solved by the provision of the embodiments characterized in the claims.

The present invention solves the technical problem because, as documented herein below and in the appended examples, DNA damage can be inflicted in a tumor/cancer cell type-specific manner, i.e. only in the tumor/cancer cells, but not (or at least to a lower degree) in immune cells (or their progenitors), like T-cells or natural killer (NK) cells (or hemocytoblasts ((omni- or multipotent) hematopoietic stem cells), common lymphoid progenitors, common myeloid progenitors, lymphoblasts or myeloblasts). More particular, it is documented herein below and in the appended examples that, one the one hand, DDIAs exist (or can be identified/screened) which target the resolution of TRCs or which induce TRCs in a tumor/cancer cell type-specific manner and, one the other hand, immune cells (or their progenitors) can be protected from a DDIA and TCRs, respectively, by (genetically) engineering them so that they are resistant against the DDIA or exhibit at least reduced susceptibility to the DDIA. The respective/resulting immune cells (or their progenitors) comprise at least one target of said DDIA which is resistant against said DDIA or has reduced susceptibility to said DDIA, for example due to an allele/a mutation, or two or more alleles/mutations, which renders/render said target as being resistant against said DDIA or as having reduced susceptibility to said DDIA.

Further, it is documented herein below and in the appended examples that, one the one hand, targets of DDIAs exist, can be generated and/or can be identified/screened, which are resistant against a DDIA or have reduced susceptibility to a DDIA and, one the other hand, that DDIAs exist, and/or can be identified/screened, which target the resolution of TRCs or which induce TRCs in tumor/cancer cells. Thus, in a hybrid therapy which relies on both, said targets and said DDIAs, the resolution of TRCs can be targeted or TRCs can be induced in a tumor/cancer cell type-specific manner, i.e. only in the tumor/cancer cells, but not (or to a lower degree) in other cells like immune cells (or their progenitors) comprising a resistant/less susceptible target of a DDIA).

Advantageous synergistic effects can be achieved on the basis of these findings:

First, effective DNA damage can be achieved in (solid) tumor/cancer cells, even in cells of (solid) tumors/cancers with large and currently unmet clinical needs (“undruggable” (solid) tumors/cancers), by applying (a) DDIA(s).

Second, the DNA damage sensitizes tumors to immune cell-mediated killing. i.e. an increased/induced immunogenic DNA damage in (solid) tumor/cancer cells) is achieved.

Third, the infiltration and activation of the immune system and the immune cell-mediated killing, respectively, can be protected form the negative impact of chemotherapeutic regimes and DDIAs, respectively. This further provides the option of, for example, increasing the number and/or amount of (an) applied DDIA(s).

In other words, during (solid) tumor/cancer chemotherapies by DDIAs, a less- or un-impaired proliferation of immune cells (or their progenitors) can be achieved and cellular immunotherapies can be empowered to attack (solid) tumors/cancers more effectively. The concomitant aid of (solid) tumor/cancer chemotherapies by anti-tumor/cancer immune cells (or their progenitors) and/or cellular immune therapies can be enhanced.

In the context of this description, TRCs are induced in tumor cells (or the resolution of TRCs is targeted) by, for example, exploiting (a) fundamental cellular process(es) that has/have not been targeted before. This inflicts high tumor/cancer cell DNA damage. At the same time, immune cells (or other cells, like immune progenitor cells) are protected from the drugs (DDIAs) used to cause these conflicts (TRCs); thereby enabling the mentioned less- or un-impaired proliferation of the (immune) cells and empower of the (immune) cells (or their progenitors) and/or cellular immune therapies to more effectively attack (solid) tumors/cancers. This inflicts highly tumor/cancer cell-specific DNA damage.

Herein, work in pancreatic carcinoma (PDAC) and metastatic colon carcinoma (CRC) is exemplarily described. While an initial work was on the pediatric tumor entity, neuroblastoma (see above, Brockmann, loc.cit.; Roeschert, loc.cit.), central mechanisms have now been identified that allow pancreatic and colon tumor cells (and others) to escape TRCs, and it was found that targeting these mechanisms causes rampant DNA damage in these entities. Importantly, not only genetic tools to decipher the molecular mechanisms are used, but also small molecule inhibitors are identified that are either available or under active development. This now enables to trigger TRCs in (solid) tumor/cancer cells and to inflict significant DNA damage in (solid) tumors/cancers with non-genotoxic molecules; especially for PDAC and CRC. Further, it was found that, although cell-intrinsic mechanisms were triggered, the resulting killing of (solid) tumor/cancer cells in vivo also depends on immune cells, in particular T cells.

Further analyses showed that the MYCN protein enables tumor cells to resolve TRCs. By now, two molecular pathways by which MYCN regulates these conflicts have been identified (see above; Roeschert loc.cit.).

Moreover, further molecular pathways by which MYCN regulates TRCs have been identified in the context of this invention. In particular, it has been shown in the context of this invention that MYCN promotes formation of a complex that restarts stalling RNA polymerase II (RNAPII). Stalling of RNAPII is frequent, for example at low nucleotide concentrations, and presents a specific challenge since stalling RNAPII moves backwards and then “normal” elongation factors cannot restart RNAPII. Recruitment of the nuclear exosome, a 3′-5′exonuclease, and a transcription factor termed TFIIS restarts RNAPII and enables it to escape co-directional TRCs.

Further, it has been shown in the context of this invention that the depletion of MYC can break the immune escape mechanisms of tumors. For example, Krenz (Cancer research, 2021, 1677) showed MYC- and MIZ1-dependent vesicular transport of double-strand RNA controls immune evasion in pancreatic ductal adenocarcinoma.

This proof-of-principle can now be used to benchmark all targeted strategies.

Further, a mouse model has been established that mimics the clinical situation and two-step surgical procedure which been proposed for metastasizing colon tumors with growth of metastases, e.g. in the liver, and to prevent postoperative (liver) failure (cf. Lang loc.cit.). When combined with a surgical strategy, e.g. the mentioned two-step surgical procedure, the molecular strategy of the invention can, on the one hand, suppress the growth of colon metastases (e.g. in the one half of the liver) while, on the other hand, allow unimpaired regeneration (e.g. in the other part of the liver). This may have the potential to cure a significant fraction of patients (even if applied only for a limited time period).

The predominant gist of the present invention is the finding that DNA damage (by (a) DDIA(s)) can be inflicted in a (solid) tumor/cancer cell type-specific manner, and that this advantageous kind of DNA damage can be combined with an improved targeted immune therapy. The most relevant means for this purpose are T cells (or other immune cells or progenitors thereof) of the invention which are resistant/less susceptible to the inhibitor(s) (DDIA(s)) which is/are used to inflict the DNA damage in the (solid) tumor/cancer). This leads to the herein provided “hybrid” (solid) tumor/cancer treatment strategy: effectively inflicting DNA damage on (solid) tumor/cancer cells while having them be attacked by less-/un-impaired T cells (and/or other immune cells (and/or while applying another cell therapy e.g. with progenitors thereof)). On this basis, the efficacy of current chemotherapies and/or cellular (immune)therapies can advantageously/synergistically be enhanced.

In the context of the present invention, the “hybrid” tumor/cancer treatment strategy is exemplarily tested by expressing the (human) Aurora-A kinase T217D/E allele/mutation in CAR T cells (or in other (immune) cells or progenitors thereof). It is found that this allele is not inhibited by, for example, LY3295668, a clinically advanced Aurora-A kinase inhibitor that is currently in clinical trials (Gong, Cancer Discovery Discov 9, 2019, 248-63). At the same time, CRISPR/Cas-mediated mutagenesis (or another method of mutagenesis) is used to identify resistance alleles/mutations for other targets and/or other respective DDIAs to be used to induce TRCs and to inflict damage in tumor/cancer cells, respectively. These resistance alleles/mutations are applied to/expressed in T cells (or in other (immune) cells or progenitors thereof) which are rendered resistant (or at least less susceptible) against the DDIA(s) or which exhibits reduced susceptibility to the DDIA(s).

Further, in the context of appended in vivo experiments, a complete tumor eradication in 25% of the animals (mice) was achieved with a combination therapy of Aurora-A and ATR inhibitors, which induces TRCs. This therapeutic success was caused, among other things, by the migration of immune cells into the tumor. This is especially promising since neuroblastoma are immunological cold tumors, meaning that they show only low immune cell infiltration.

However, the immune system was not able to completely eliminate the tumor in 75% of the mice, causing death of the mice after the end of therapy. Tumor re-growth can be explained by the situation that the immune cells are also attacked by the inhibitor and therefor are not able to completely eradicate the tumor cells. In the context of the invention, immune cells are provided that are resistant against the inhibitor and are, for example, able to further/completely eradicate the tumor cells.

In one aspect, the present invention relates to a novel hybrid treatment, in particular of a (solid) tumor/cancer. Said treatment comprises (i) targeting (by a DDIA) the resolution TRCs, in particular in ((a) cell(s) of) a/said (solid) tumor/cancer; or (ii) inducing (by a DDIA) TRCs, in particular in ((a) cell(s) of) a/said (solid) tumor/cancer. This is the chemotherapeutic aspect of the invention. The hybrid treatment according to the invention further comprises the use/administration of a cell, in particular of an immune cell, or a progenitor cell thereof, which is resistant against said targeting/inducing (i.e. against a/the DDIA) or which exhibits reduced susceptibility to said targeting/inducing (i.e. to a/the DDIA). The immune cell, or progenitor cell thereof, is envisaged to target (a/said cell(s) of) a/said (solid) tumor/cancer. This is the immunotherapeutic aspect of the invention; i.e. the aspect of targeted tumor/cancer immunotherapy, such as adoptive T-cell therapy.

In another aspect, the present invention relates to a cell, in particular to an immune cell, or a progenitor cell thereof, which is resistant against a DDIA or which exhibits reduced susceptibility to a DDIA.

In principle, any immune cell, or progenitor cell thereof, may be provided and used in accordance with the invention. Respective immune cells, and progenitors thereof, are well known in the art. These are, for example, described and depicted under https://en.wikipedia.org/wiki/Haematopoiesis (Jun. 26, 2021) or https://www.thermofisher.com/de/de/home/life-science/antibodies/antibodies-learning-center/antibodies-resource-library/cell-signaling-pathways/hematopoiesis-pluripotent-stem-cells.html (Jun. 26, 2021)). A respective exemplary overview of the cells (e.g. immune cells, or progenitor cell thereof) which may be provided and used in accordance with the invention, is given by the appended FIG. 9 (extracted from https://en.wikipedia.org/wiki/Haematopoiesis; on Jun. 26, 2021). Any of the cells depicted in FIG. 9, in particular any of the depicted immune cells, or progenitors thereof, is, in principle, envisaged to be provided/used in accordance with the invention.

An immune cell which is provided and/or used according to the invention may, in principle, be an immune cell selected from the group consisting of T cells/T-lymphocytes (see, for example, Newick, Annual review of medicine 68, 2017, 139-52), NK cells (also known in the art as large granular lymphocytes; see, for example, Xie, EBioMedicine 59, 2020, 102975), small lymphocytes, B cells/B lymphocytes, plasma cells, lymphoid dendritic cells, macrophages, myeloid dendritic cells and mast cells. Particularly preferred (immune) cells provided and/or used according to the invention are those which are capable of (specifically) recognizing and/or attacking/eliminating tumor/cancer cells. In particular, an immune cell which is provided and/or used according to the invention is a lymphocyte, preferably a primary lymphocyte, more preferably a T-cell, more preferably a primary T-cell.

It is generally preferred that a cell which is provided/used in accordance with the invention is a human cell. In principle, however, other non-human cells are not excluded). Thus, it is most preferred that a cell provided/used herein is a human lymphocyte, more preferably a primary human lymphocyte, more preferably a primary human T-cell.

The term “primary” and analogous terms in reference to a cell or cell population as used herein correspond to their commonly understood meaning in the art, i.e., referring to cells that have been obtained directly from living tissue (e.g. a biopsy such as a blood sample) or from a subject, which cells have not been passaged in culture, or have been passaged and maintained in culture but without immortalization.

The cell, in particular lymphocyte, according to the invention can be any cell/lymphocyte described herein or known in the art to be suitable for use, in particular in an (adoptive)(immune) cell therapy (e.g. the immunotherapeutic aspect of the invention). However, it is recognized that the means and methods of the invention may also be applicable for uses outside of therapies, such as in screening methods and/or in model systems, e.g. for use in in vitro screenings/assays or in vivo animal models, or screening methods using these. Therefore, the invention also encompasses (genetically engineered) non-human or human cells/lymphocytes and/or (genetically engineered) cells/lymphocytes derived from cell lines, which may be of human or non-human origin.

Non-limiting examples of lymphocytes (which may be primary lymphocytes or derived from cell lines) include NK cells, inflammatory T-lymphocytes, cytotoxic T-lymphocytes, helper T-lymphocytes, CD3+T lymphocytes, CD4+T lymphocytes, CD8+T lymphocytes, γδ T lymphocytes, invariant T lymphocytes and NK T lymphocytes. It is preferred that the cell of the invention is a (genetically engineered) (primary) NK cell or, more preferably, a (primary) T cell, preferably a human cell, more preferably a (primary) human NK or T cell, and most preferably a (primary) human T cell. AT cell described herein may be, e.g. a CD3+ T cell, CD8+ T cell, a CD4+-T cell, or γδ T cell.

As known in the art, T cells are cells of the adaptive immune system that recognize their target in an antigen specific manner. Typically, these cells are characterized by surface expression of CD3 and a T cell receptor (TCR), which recognizes a cognate antigen in the context of a major histocompatibility complex (MHC). CD4+ T cells recognize an antigen through their TCR in the context of MHC class II molecules that are predominantly expressed by antigen-presenting cells. CD8+ T cells recognize their antigen in the context of MHC class I molecules that are present on most cells of the human body. Methods for identifying, separating and maintaining specific subpopulations of T cells (e.g. as a culture of primary T cells) such as CD3+, CD4+ and/or CD8+ T cells from a cell population (such as a population of peripheral blood mononuclear cells e.g. having been isolated from a patient for the purpose of autologous cell therapy) are well known to those skilled in the art and include flow cytometry, microscopy, immunohistochemistry, RT-PCR or western blot (Kobold, J Natl Cancer Inst 107(2015), 107).

A particular example of a progenitor cell which is provided and/or used according to the invention is a progenitor cell selected from the group consisting of hemocytoblasts ((omni- or multipotent) hematopoietic stem cells), common lymphoid progenitors, common myeloid progenitors, lymphoblasts, myeloblasts of monoblasts. A preferred particular example of a progenitor which is provided and/or used according to the invention is a progenitor cell selected from the group consisting of common lymphoid progenitors, lymphoblasts, prolymphocytes and small lymphocytes.

An immune cell, or a progenitor cell thereof, which is provided and/or used according to the invention may be a cell (preferably an immune cell), or a progenitor cell thereof, of the granulopoiesis, monocytopoiesis or, preferably, lymphopoiesis (for example FIG. 9, 4^{th}, 5^{th} and 6^{th} lines from the left), or may be a mast cell (for example FIG. 9; 3^{rd} line from the left). Examples of these cells, or of progenitors thereof, are depicted in FIG. 9, 4^{th}, 5^{th} and 6^{th} lines from the left.

In principle, a cell, or a progenitor thereof, which is provided and/or used according to the invention may also be a cell, or a progenitor thereof, of the thrombopoiesis or erythropoiesis (for example, FIG. 10, 1^{st} and 2^{nd} lines from the left). However, cells of the granulopoiesis, monocytopoiesis or lymphopoiesis (cf., for example FIG. 10, 4^{th}, 5^{th} and 6^{th} lines from the left), or mast cells (cf., for example FIG. 10; 3^{rd} line from the left) are preferred (see above).

In principle, a (omni- or multipotent) stem cell may also be a cell provided and/or used according to the invention (i.e. being resistant against a DDIA or exhibiting reduced susceptibility to a DDIA). Among the cells provided and/or used according to the invention, immune cell progenitors are preferred, and immune cells are even more preferred.

The cell (in particular the immune cell or progenitor thereof) as provided and/or used according to the invention may be a proliferative/proliferating cell, e.g. a proliferative/proliferating immune cell, a (committed) progenitor cell or a stem cell. As mentioned, such cells can also advantageously be used in (immune) therapy (e.g. the one aspect of the hybrid tumor/cancer therapy), for example in combination with a (solid) tumor/cancer (chemo)therapy (like a (chemo)therapy as described herein (e.g. the other aspect of the hybrid tumor/cancer therapy). Thus, it is particularly advantageous if such cells are resistant against a DDIA or exhibit reduced susceptibility to a DDIA in accordance with the invention.

The (genetically engineered T-) cell of the invention may (further) comprise, e.g. be further engineered with additional nucleic acid molecules to express (in addition to the target with resistancy/less susceptibility to (a) DDIA(s), (an)other polypeptide(s) of use in (immune) cell therapy (e.g. in the one aspect of the hybrid tumor/cancer therapy). Another polypeptide to be expressed may be a (exogenous) T cell receptor, a (exogenous) chimeric antigen receptor (CAR) (e.g. specific for a tumor/cancer of interest), a (exogenous) cytokine receptor (which sequence may or may not be modified relative to the endogenous/wild-type sequence), and/or an endogenous cytokine receptor having a sequence modified relative to the wild-type sequence (i.e a modified endogenous cytokine receptor). Alternately or additionally, the (T-) cell of the invention can be further (genetically) modified to disrupt an/the expression of the endogenous ((T-) cell) receptor, such that it is not expressed or expressed at a reduced level as compared to a (T-) cell absent such modification.

As used herein, the term “reduced expression” and analogous terms refer to any reduction in the expression (e.g. of the endogenous (T cell) receptor) at the cell surface of a (genetically modified) cell when compared to a control cell. The term “reduced” can also refer to a reduction in the percentage of cells in a population of cells that express an endogenous polypeptide (i.e., an endogenous (T cell) receptor) at the cell surface when compared to a population of control cells. Accordingly, the term “reduced expression” (e.g. in connection with the expression of an endogenous (T cell) receptor) encompasses both, a partial knockdown and a complete knockdown (e.g. of the endogenous (T cell) receptor) within the population of (genetically modified) cells. For example, “reduced” means≤5%, ≤10%, ≤20%, ≤30%, ≤40%, ≤50%, ≤75%, ≤90% expression as compared to a control cell.

The cell (in particular the immune cell) provided and/or used according to the invention may comprise/express a (recombinant) (exogenous) T-cell receptor or an artificial T-cell receptor (or both). The (exogenous) artificial T-cell receptor may be generated by recombinant techniques and may, thus, also be termed recombinant (exogenous) artificial T-cell receptor. Recombinant T-cell receptors and artificial T-cell receptors, their (recombinant) generation and means and methods for generating cells that express the same (e.g. on the basis of (genetical) engineering) are well known in the art (see above).

As used herein, an “exogenous (T cell) receptor” refers to receptor whose sequence is introduced into the genome of a cell/lymphocyte (e.g. a human (primary) T cell) that may or may not endogenously express the receptor. Expression of an exogenous (T cell) receptor on an immune effector cell can confer specificity for a specific epitope or antigen (e.g. an epitope or antigen preferentially present on the surface of a tumor/cancer cell or other disease-causing cell). Such exogenous (T cell) receptors can comprise alpha and beta chains or, alternatively, may comprise gamma and delta chains. Exogenous (T cell) receptors useful in the invention may have specificity to any antigen or epitope of interest. Examples of such exogenous (TC)Rs include, but are not limited to, receptors recognizing WT1 (Wilms tumor specific antigen 1; see, e.g. Sugiyama, Japanese Journal of Clinical Oncology 40, 2010, 377-87); receptors recognizing MAGE (see, e.g. WO 2007/032255), receptors recognizing SSX (see, e.g. Zhou, J. Natl. Cancer Inst. 97, 2005, 823-835), receptors recognizing NY-ESO-1 (see, e.g. WO 2005/113595), receptors recognizing HER2neu (see, e.g. WO 2011/0280894) and receptors recognizing other tumor/cancer antigens which are known to the skilled person (for example as disclosed in June, Science 359, 2018, 1361-5).

In a preferred embodiment, the cell (in particular the immune cell) provided and/or used according to the invention comprises/expresses a chimeric antigen receptor (CAR). CARs, their (recombinant) generation and means and methods for generating cells that express the same (e.g. on the basis of (genetical) engineering) are well known in the art (see below and, e.g., Hudecek loc.cit.; Wallstabe loc.cit.; Pommersberger, Current Protocols in Immunology 128, 2020).

CAR refers to an engineered receptor that confers or grafts specificity for an antigen onto a cell, in particular a lymphocyte (e.g. most preferably a (human) (primary) T cell). A CAR typically comprises an extracellular ligand-binding domain or moiety and an intracellular domain that may comprise one or more stimulatory domain(s) that transduce the signals necessary for cell/lymphocyte (e.g. T cell) activation. In some embodiments, the extracellular ligand-binding domain or moiety can be in the form of single-chain variable fragments derived from a monoclonal antibody (scFvs), which provide specificity for a particular epitope or antigen (e.g. an epitope or antigen associated with cancer, such as preferentially express on the surface of a cancer cell or other disease-causing cell). The extracellular ligand-binding domain can be specific for any antigen or epitope of interest. The intracellular stimulatory domain typically comprises the intracellular domain signaling domains of non-TCR T cell stimulatory/agonistic receptors. Such cytoplasmic signaling domains may include, for example, but not limited to, the intracellular signaling domain of CD3ζ, CD28, 4-1BB, OX40, or a combination thereof. A chimeric antigen receptor may further include additional structural elements, including a transmembrane domain that is attached to the extracellular ligand-binding domain via a hinge or spacer sequence.

As with the optionally engineered exogenous TCR, the optional CAR may provide tumor/cancer specificity and allow for the recognition target tumor/cancer or disease cells. Suitable CARs are well known in the art, and include, but are not limited to, anti-EGFRv3-CAR (see, e.g. WO 2012/138475), anti-CD22-CAR (see, e.g. WO 2013/059593), anti-BCMA-CAR (see, e.g. WO 2013/154760), anti-CD19-CAR (see, e.g. WO 2012/079000), anti-CD123-CAR (see, e.g. US 2014/0271582), anti-CD30-CAR (see, e.g. WO 2015/028444), anti-Mesothelin-CAR (see, e.g. WO 2013/142034) and a CAR which binds to an other tumor/cancer antigen which are known to the skilled person (for example as disclosed in June loc. cit.).

The (genetically) engineered/modified cell of the invention, may further (engineered/modified to) express still other (exogenous) cytokine receptor (which may be a wild-type sequence or may have an amino acid sequence modified relative to that of the endogenous/wild type sequence) and/or an endogenous cytokine receptor having a sequence modified from that of the endogenous sequence. As used herein, an “exogenous cytokine receptor” refers to a cytokine receptor whose sequence is introduced into the genome of a lymphocyte (e.g. a human primary T cell) that may or may not endogenously express the receptor. Similarly, “endogenous cytokine receptor” refers to a receptor whose sequence is introduced into the genome of a lymphocyte (e.g. a human primary T cell) that endogenously expresses the receptor. The introduced exogenous or endogenous cytokine receptor may be modified to alter the function of the receptor normally exhibited in its endogenous system, e.g. to provide for dominant-negative receptors (receptors that bind to ligands, but which binding does not elicit endogenous activity). Expression of an exogenous cytokine receptors (modified or not) and/or modified endogenous receptors can confer ligand-specific activity not normally exhibited by the lymphocyte or, in the case of dominant-negative modifications, can act a ligand-sinks to bind cytokines and prevent and/or decrease the ligand-specific activity. One such dominant-negative receptor known in this respect is the dominant-negative TGF-β receptor 2 (DNR; SEQ ID NO:6), a modified TGF-β receptor 2 lacking the intracellular domain of the endogenous molecule which prevents the signal transduction into the cell on TGF-β binding; see, Siegel, PNAS 100, 2003, 8430-8435.

It is preferred in the context of the present invention that the receptor (e.g. recombinant T-cell receptor, artificial T-cell receptor, CAR) to be comprised/expressed by (and on the surface of) the cell (in particular the immune cell) as provided and/or used herein specifically binds to a tumor/cancer antigen. The tumor/cancer antigen may be a tumor-specific antigen (TSA) or to a tumor-associated antigen (TAA). Respective antigens are well known in the art and are, for example, described in DeSelm (Journal of surgical oncology 116, 2017, 63-74), Newick (loc. cit.) and June (loc. cit.). Human tumor/cancer antigens are preferred as antigens to be recognized by the receptors employed in accordance with the invention.

Two examples of (tumor/cancer) antigens to be recognized by the receptors as employed in the context of the invention are the human antigens B7H3 (preferred), mesothelin and ROR1. These antigens are well known and characterized in the art (Hudecek, Blood 116, 2010, 4532-41; Wallstabe, JCI insight 4, 2019; Klampatsa, Expert Opin Biol Ther 21, 2021, 473-86; Majzner, Clin Cancer Res 25, 2019, 2560-74). These antigens are particularly useful in the models/model systems and in the screening methods provided in the context of the invention (see below). In a most preferred, however non-limiting embodiment, the immune cell provided and/or used according to the invention is a CAR T-cell. CAR T-cells which are resistant against a DDIA to be used (in accordance with the invention), or which have reduced susceptibility to said DDIA, are particularly useful in the (hybrid) tumor/cancer treatments of the invention and as described herein. Means and methods for generating CAR T-cells, for example by (technical/genetical) engineering and respective (recombinant) techniques are well known in the art and are, for example, described in Hudecek (loc.cit.), Wallstabe (loc.cit.) and Pommersberger (loc.cit.). For example, CAR T-cells may be generated by lentiviral transduction (cf., for example, Pommersberger (loc.cit.).

Further, means and methods for making a cell, e.g. an immune cell or progenitor cell thereof according to the invention, resistant against a DDIA, or as having reduced susceptibility to said DDIA, are also known in the art (e.g. Neggers, Nat. Commun 9(1), 2018, 502; Cluse loc.cit.; Pommersberger loc.cit.). They are also described herein and in the appended examples (e.g. Example 1 and Example 7). Most relevantly, for this purpose, a target of a DDIA with resistancy against, or reduced susceptibility to, the DDIA may be introduced into the cell; or an allele and/or mutation which confers resistancy against, or reduced susceptibility to, the DDIA on a target within the cell may be introduced into the target and cell, respectively. For example, respective (mutagenized/engineered) targets, and (a pool of) respective cells comprising it, may be generated by using CRISPR-Cas mutagenesis. Respective methods are described in, for example, Neggers (loc.cit.). Transduction, e.g. lentiviral transduction, may be applied in the context of generating the cells of the invention; e.g. for introducing the (mutagenized/engineered) targets into (a pool of) respective cells (see, for example, Prommesberger, loc.cit.). (Pools of) cells may be screened for target identification or drug sensitivity/DDIA sensitivity as, for example, described in Neggers (loc.cit.), Cluse (loc. it.) or Barretina (Nature 483 (7391), 2012, 603-7). Stimulation of (immune) cells, in particular T-cells, and the measurement of their functionality may also be performed in this respect; as, for example, described in Reinwald (The Journal of Immunology 180(9), 2008, 5890-7). Further, measurements of (immune) cell proliferation may be performed; as, for example, described in Quah (Nat. Protoc. 2, 2007, 2049-56).

Several targets of DDIAs with resistancy against, or reduced susceptibility to, a DDIA are also known in the art (e.g. Roeschert loc.cit.; Brockmann loc.cit.; Sloane loc.cit.). Likewise known in the art are alleles and/or mutations which confer resistancy against or reduced susceptibility to a DDIA on a target (e.g. Roeschert loc.cit.; Brockmann loc.cit.; Sloane loc.cit.). Examples of suitable potential targets are listed under item 26, infra. Other targets of DDIAs with resistancy against, or reduced susceptibility to, a DDIA may also be screened/identified by appropriate screening methods (for example as listed under items 34, infra). A prominent, however non-limiting, example of a DDIA resistant/less susceptible target is the (human) Aurora A kinase T217D or T217E mutant. The respective allele is the (human) Aurora A kinase T217D or T217E allel. The respective mutation is the Aurora A kinase T217D or T217E mutation (in mouse Aurora A kinase/AURKA, the corresponding mutations are the T208D or T208E mutations, respectively; examples of database entries of the respective non-mutated human and mouse Aurora A kinases are Genbank AAH02499.1 and NP_035627.1 respectively.). Respective DDIAs are, for example, MLN8054, MLN8237 and LY3295668. Exemplary reference is made in this respect to Roeschert (loc.cit.), Brockmann (loc.cit.), Sloane (loc.cit.), Gang (loc.cit.) and Du (Molecular Cancer 20(15), 2021, 1-27). Particular Aurora A kinases inhibitors that may be used in accordance with the invention are disclosed in Du (loc.cit.), in particular Table 3 and Table 4 thereof (“Compound names” and “Drug name”, respectively).

In principle, any target of a DDIA (e.g. to be used in the chemotherapeutic aspect of the hybrid therapy) may be used in accordance with the invention in a form/version which is resistant against the DDIA, or which has reduced susceptibility to the DDIA, in the (immune) cells/progenitors of the invention (e.g. in the context of the aspect of (immune) cell therapy of the hybrid therapy). Thus, any target of a DDIA which is described herein elsewhere, in particular herein below, may be used in a resistant/less susceptible form/version in the (immune) cells/progenitors of the invention.

Some of the known DDIAs (e.g. PARP inhibitors) work by trapping the target (e.g. PARP) on DNA. Thus, a cell with an (engineered) deletion/depletion of such a target (e.g. PARP) is largely resistant/less susceptible against such DDIAs. Thus, in one embodiment, a cell, e.g. an immune cell or progenitor cell thereof according to the invention, is made resistant against such DDIAs, or as having reduced susceptibility to such DDIAs, by deletion/depletion of such a target (i.e. a target to be trapped on DNA, e.g. PARP) in the cell.

Deletion/depletion can, for example, be achieved by inhibiting/reducing the expression of a target (e.g. on transcription and/or translation level). Respective means and methods are known in the art. These comprise, for example, the use of siRNA/RNAi or small hairpin RNA (shRNA) approaches (e.g. Cluse loc.cit.; Example 1; Example 4). The deletion/depletion (e.g. by shRNA) may be inducible, for example as described herein elsewhere (e.g. by doxycycline, or comparable means/methods; e.g. Example 1; Example 4). What has been said above with respect to “reduced expression” and related terms may also apply here, mutatis mutandis.

Means and methods for generating a cell, e.g. an immune cell or progenitor cell thereof according to the invention are well known in the art and are also described herein elsewhere and in the appended examples. Most relevantly, these means and methods may comprise engineering, in particular technical engineering, more particular genetical engineering. Respective (technical/genetical) engineering means and methods are known in the art and are also described herein and in the appended examples (see above and, for example, Example 1 and Example 7).

It is particularly envisaged in the context of the invention that the cells of the invention are genetically engineered so as to express a resistant/less susceptible target of a DDIA (e.g. as described herein) and, optionally, a further receptor (e.g. T-cell receptor, CAR or another receptor as described herein).

The genetically engineered cell of the invention may either be a directly genetically engineered cell (e.g. lymphocyte), i.e., a cell (e.g. lymphocyte) that has been directly subject to genetic engineering methods, or may be a cell (e.g. lymphocyte) derived from such a cell (e.g. lymphocyte), e.g. a daughter cell or progeny of a cell (e.g. lymphocyte) that was directly genetically engineered. Thus, the genetically engineered cell (e.g. lymphocyte) of the invention may be a directly genetically engineered cell (e.g. lymphocyte) as well as any cell derived therefrom, such as a daughter cell obtained by culture of the directly engineered/modified cell (e.g. lymphocyte).

The genetically engineered cell of the invention (e.g. lymphocyte) may transiently or stably express the resistant/less susceptible target of a DDIA and the optional receptor. Additionally, the expression can be constitutive or constitutional, depending on the system used as is known in the art. (A) respective encoding nucleic acid(s) may or may not be stably integrated into the engineered cell's genome. Methods for achieving stable integration of introduced nucleic acids encoding desired proteins are well known in the art, and the invention encompasses the use of such methods as well as those described herein. Preferably, the herein provided cell (e.g. lymphocyte, preferably human lymphocyte, more preferably primary human lymphocyte, and more preferably (primary) human T cell) has been genetically modified by introducing (a) respective encoding nucleic acid molecule(s) into the cell by using a viral vector (e.g. a retroviral vector or a lentiviral vector; see, for example, Pommersberger (loc.cit.)).

Methods for genetically engineering cells (in particular lymphocytes such as T cells and NK cells) to express polypeptides of interest (e.g. the resistant/less susceptible targets and/or (cell surface) receptors) are known in the art and can generally be divided into physical, chemical, and biological methods. The appropriate method for given cell type and intended use can readily be determined by the skilled person using common general knowledge. Such methods for genetically engineering cells by introduction of nucleic acid molecules/sequences encoding the polypeptide of interest (e.g. in an expression vector) include but are not limited to chemical- and electro-poration methods, calcium phosphate methods, cationic lipid methods, and liposome methods. The nucleic acid molecule/sequence to be transduced can be conventionally and highly efficiently transduced by using a commercially available transfection reagent and/or by any suitable method known in the art or described herein. In addition to methods of genetically engineering cells with nucleic acid molecules comprising or consisting of DNA sequences, the methods disclosed herein can also be performed with mRNA transfection. “mRNA transfection” refers to a method well known to those skilled in the art to transiently express (a) protein(s) of interest, in the present case the resistant/less susceptible target and/or receptor, in a cell, preferably lymphocyte (e.g. T cell). Accordingly, the methods herein may be used to genetically engineer a cell (e.g. lymphocyte) to transiently or stably (either constitutively or conditionally) express the polypeptide(s) of interest. For example, with respect to mRNA transfection, cells (e.g. lymphocytes) may be electroporated with the mRNA coding for the resistant/less susceptible target and/or receptor as described herein by using an electroporation system (such as e.g. Gene Pulser, Bio-Rad) and thereafter cultured by standard cell culture protocols (see, e.g. Zhao, Mol Ther. 13(2006), 151-159). Useful, but non-limiting, methods for genetically engineering cells (in particular lymphocytes such as T cells and NK cells) to express polypeptides of interest (e.g. the resistant/less susceptible targets and/or (cell surface) receptors) are those which make use of transposons, like methods based on the Sleeping Beauty system (e.g. as described in Monjezi, Leukemia 31, 2017, 186-94).

Physical methods for introducing a polynucleotide into a host cell include calcium phosphate precipitation, lipofection, particle bombardment, microinjection, electroporation, and the like; see, e.g. Sambrook, 2012, Molecular Cloning: A Laboratory Manual, volumes 1-4, Cold Spring Harbor Press, NY.

Biological methods for introducing a polynucleotide of interest into a host cell include the use of DNA and RNA vectors. Viral vectors, and especially retroviral vectors, have become the most widely used method for inserting genes into mammalian cells or lentiviral (e.g., human cells such as T cells). Accordingly, retroviral or lentiviral vectors may be used in the methods and cells disclosed herein. Viral vectors, however, can be derived from a variety of different viruses, including but not limited to lentivirus, poxviruses, herpes simplex virus I, adenoviruses and adeno-associated viruses; see, e.g. U.S. Pat. Nos. 5,350,674 and 5,585,362. Non-limiting examples of suitable retroviral vectors for transducing cells (e.g. T cells) include SAMEN CMV/SRa (Clay, J. Immunol. 163(1999), 507-513), LZRS-id3-IHRES (Heemskerk, J. Exp. Med. 186(1997), 1597-1602), FeLV (Neil, Nature 308(1984), 814-820), SAX (Kantoff, Proc. Natl. Acad. Sci. USA 83(1986), 6563-6567), pDOL (Desiderio, J. Exp. Med. 167(1988), 372-388), N2 (Kasid, Proc. Natl. Acad. Sci. USA 87(1990), 473-477), LNL6 (Tiberghien, Blood 84(1994), 1333-1341), pZipNEO (Chen, J. Immunol. 153(1994), 3630-3638), LASN (Mullen, Hum. Gene Ther. 7(1996), 1123-1129), pG1XsNa (Taylor, J. Exp. Med. 184(1996), 2031-2036), LCNX (Sun, Hum. Gene Ther. 8(1997), 1041-1048), SFG (Gallardo, Blood 90(1997), LXSN (Sun, Hum. Gene Ther. 8(1997), 1041-1048), SFG (Gallardo, Blood 90(1997), 952-957), HMB-Hb-Hu (Vieillard, Proc. Natl. Acad. Sci. USA 94(1997), 11595-11600), pMV7 (Cochlovius, Cancer Immunol. Immunother. 46(1998), 61-66), pSTITCH (Weitjens, Gene Ther 5(1998), 1195-1203), pLZR (Yang, Hum. Gene Ther. 10(1999), 123-132), pBAG (Wu, Hum. Gene Ther. 10(1999), 977-982), rKat.43.267bn (Gilham, J. Immunother. 25(2002), 139-151), pLGSN (Engels, Hum. Gene Ther. 14(2003), 1155-1168), pMP71 (Engels, Hum. Gene Ther. 14(2003), 1155-1168), pGCSAM (Morgan, J. Immunol. 171(2003), 3287-3295), pMSGV (Zhao, J. Immunol. 174(2005), 4415-4423), or pMX (de Witte, J. Immunol. 181(2008), 5128-5136). Most preferred are lentiviral vectors. Non-limiting examples of suitable lentiviral vectors for transducing T cells are, e.g. PL-SIN lentiviral vector (Hotta, Nat Methods. 6(2009), 370-376), p156RRL-sinPPT-CMV-GFP-PRE/Nhel (Campeau, PLoS One 4(2009), e6529), pCMVR8.74 (Addgene Catalogoue No.:22036), FUGW (Lois, Science 295(2002), 868-872, pLVX-EF1 (Addgene Catalogue No.: 64368), pLVE (Brunger, Proc Natl Acad Sci USA 111(2014), E798-806), pCDH1-MCS1-EF1 (Hu, Mol Cancer Res. 7(2009), 1756-1770), pSLIK (Wang, Nat Cell Biol. 16(2014), 345-356), pLJM1 (Solomon, Nat Genet. 45(2013), 1428-30), pLX302 (Kang, Sci Signal. 6(2013), rs13), pHR-IG (Xie, J Cereb Blood Flow Metab. 33(2013), 1875-85), pRRLSIN (Addgene Catalogoue No.: 62053), pLS (Miyoshi, J Virol. 72(1998), 8150-8157), pLL3.7 (Lazebnik, J Biol Chem. 283(2008), 11078-82), FRIG (Raissi, Mol Cell Neurosci. 57(2013), 23-32), pWPT (Ritz-Laser, Diabetologia. 46(2003), 810-821), pBOB (Marr, J Mol Neurosci. 22(2004), 5-11), and pLEX (Addgene Catalogue No.: 27976). A non-limiting example of a suitable lentiviral vector is disclosed in Pommersberger (loc.cit.).

The invention also encompasses vectors comprising nucleic acid molecules encoding the resistant/less susceptible target and/or receptor described herein. As used herein, the term “vector” relates to a circular or linear nucleic acid molecule that can autonomously replicate in a host into which it has been introduced. The term “vector” as used herein particularly refers to a plasmid, a cosmid, a virus, a bacteriophage and other vectors commonly used in genetic engineering as described herein or as is known in the art. Preferably, the disclosed vectors are suitable for the transformation of cells, like, for example lymphocytes, preferably human lymphocytes and more preferably human primary lymphocytes, including but not limited to NK cells and T cells such as CD8+ T cells, CD4+ T cells, CD3+ T cells, γδ T cells, invariant T cells and NK T cells. Vectors of use in connection with the present invention comprise a nucleic acid sequence encoding the full-length target and/or peptide, or (a) functional variant(s) thereof, like, for example, (a) functional fragment(s).

It will be appreciated that the vectors disclosed herein may contain additional sequences to allow function such as replication or expression of a desired sequence in the cell system. For example, the vectors may comprise the nucleic acid molecule encoding the target and/or receptor, or for (a) functional variant(s) thereof, under the control of regulatory sequences. The term “regulatory sequence” refers to DNA sequences that are necessary to effect the expression of coding sequences to which they are operably linked. As is understood in the art, the nature of such control sequences differs depending upon the host organism. In prokaryotes, control sequences generally include promoters, ribosomal binding sites, and terminators. In eukaryotes control sequences generally include promoters, terminators and, in some instances, enhancers, sequences encoding transactivators and/or transcription factors. The term “control sequence” is intended to include, at a minimum, all components the presence of which are necessary for expression, and may also include additional advantageous components, e.g., to allow replication. Regulatory or control sequences (including but not limited to promoters, transcriptional enhancers and/or sequences), which allow for induced or constitutive expression of the target and/or receptor, or its variant or fragment, as described herein, may be employed. Suitable promoters include but are not limited to the CMV promoter, the UBC promoter, PGK, the EF1A promoter, the CAGG promoter, the SV40 promoter, the COPIA promoter, the ACT5C promoter, or the TRE promoter (e.g., as disclosed in Qin, PLoS One. 5(2010), e10611); the Oct3/4 promoter (e.g., as disclosed in Chang, Molecular Therapy 9(2004), S367-S367 (doi: 10.1016/j.ymthe.2004.06.904)); or the Nanog promoter (e.g., as disclosed in Wu, Cell Res. 15(2005), 317-24).

The vectors of use in the present invention are preferably expression vectors. Suitable expression vectors have been widely described in the literature and the determination of the appropriate expression vector can be readily made by the skilled person using routine methods. Preferably, the vectors disclosed herein comprises a recombinant polynucleotide (i.e., a nucleic acid sequence encoding the target and/or receptor, or (a) functional variant(s)) as well as expression control sequences operably linked to the nucleotide sequence to be expressed. The herein described vectors may also comprise a selection marker gene and a replication-origin ensuring replication in the host (i.e. a genetically engineered (e.g., transduced) cell, like, for example, lymphocyte such as a T cell). Moreover, the herein provided vectors may also comprise a termination signal for transcription. Between the promoter and the termination signal may be at least one restriction site or a polylinker to enable the insertion of a nucleic acid molecule encoding a polypeptide desired to be expressed (e.g. a nucleic acid sequence encoding the target and/or (a) functional variant(s) thereof). The use of expression vectors, including insertion of the encoding nucleic acid molecule/sequence and the harvest of the expressed polypeptide, is routine in the art. Non-limiting examples of vectors suitable for use in the present invention include cosmids, plasmids (e.g., naked or contained in liposomes) and viruses (e.g., lentiviruses, retroviruses, adenoviruses, and adeno-associated viruses) that incorporate the nucleic acid molecules encoding CCR8, or a functional variant or fragment thereof. Of preferred use is a viral vector, especially a lentiviral vector.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). Alternately, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids may be naturally occurring or synthetic lipids. Lipids suitable for use in methods of nucleic acid molecule delivery to a host cell (i.e., to genetically engineer the host cell) can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.).

Regardless of the method used to introduce exogenous nucleic acids into a host cell, in order to confirm the presence of the recombinant DNA sequence in the target cell (i.e., to confirm that the cell has been genetically engineered according to the methods disclosed herein), a variety of assays may be performed. Such assays include, for example, “molecular biological” assays well known to those of skill in the art, such as Southern and Northern blotting, RT-PCR, PCR and sequencing; “biochemical” assays, such as detecting the presence or absence of a particular polypeptide, e.g. by immunological means (ELISAs and/or Immuno blots) or by assays described herein to identify whether the cell exhibits a property or activity associated with the engineered polypeptide, i.e. assays to assess whether the lymphocyte (more preferably a human primary lymphocyte such as an NK cell or T cell) exhibits CCR8 activity.

The genetic engineering methods disclosed herein may be applied to the (immune) cells or progenitors of the invention, for example, lymphocytes (e.g. T cells or NK cells).

As described herein, the genetically engineered cell (e.g. lymphocyte) of the present invention may be recombinantly modified with a nucleic acid sequence encoding (and driving/permitting expression of) the herein described resistant/less susceptible target and/or receptor, or (a) functional variant(s) thereof. In the case of cells bearing natural anti-tumor specificity (such as tumor-infiltrating lymphocytes (TIL see, e.g. Dudley, J Clin Oncol. 31(2013), 2152-2159)) or antigen-specific cells sorted from the peripheral blood of patients for their tumor-specificity by flow cytometry (Hunsucker, Cancer Immunol Res. 3(2015), 228-235), the genetically engineered cells described herein may only be modified to express the target, or (the) functional variant(s) thereof.

The cells, in particular the (primary) lymphocytes described herein can be isolated and/or obtained from a number of tissue sources, including but not limited to, peripheral blood mononuclear cells isolated from a blood sample, bone marrow, lymph node tissue, cord blood, thymus tissue, tissue from a site of infection, ascites, pleural effusion, spleen tissue, and/or tumors by any method known in the art or described herein. In a non-limiting example in the context of a T cell, a genetically engineered primary T cell of the present invention is that having been obtained and/or isolated from a T cell population from subject (preferably a human patient). Methods for isolating/obtaining specific populations of lymphocytes (including T cells) from patients or from donors are well known in the art and include as a first step, for example, isolation/obtaining a donor or patient sample known or expected to contain such cells, e.g. a blood or bone marrow sample. After isolating/obtaining the sample, the desired cells, e.g. NK cells or T cells, are separated from the other components in the sample. Methods for separating a specific population of desired cells from the sample are known and include, but are not limited to, e.g. leukapheresis for obtaining T cells from the peripheral blood sample from a patient or from a donor; isolating/obtaining specific populations from the sample using a FACSort apparatus; and selecting specific populations from fresh biopsy specimens comprising living lymphocytes by hand or by using a micromanipulator (see, e.g. Dudley, Immunother. 26(2003), 332-342; Robbins, Clin. Oncol. 29(20011), 917-924; Leisegang, J. Mol. Med. 86(2008), 573-58). The term “fresh biopsy specimens” refers to a tissue sample (e.g. a tumor tissue or blood sample) that has been or is to be removed and/or isolated from a subject by surgical or any other known means. The isolated/obtained cells are subsequently cultured and expanded according to routine methods known in the art for maintaining and/or expanding the desired primary cell and/or primary cell population. For example, in the context of T cells, culture may occur in the presence of an anti-CD3 antibody; in the presence of a combination of anti-CD3 and anti-CD28 monoclonal antibodies; and/or in the present of an anti-CD3 antibody, an anti-CD28 antibody and one or more cytokines, e.g. interleukin-2 (IL-2) and/or interleukin-15 (IL-15) (see, e.g. Dudley, Immunother. 26(2003), 332-342; Dudley, Clin. Oncol. 26(2008), 5233-5239).

As is well known in the art, it is also possible to isolate/obtain and culture/select one or more specific sub-populations of T cells, which methods are also encompassed by the invention. Such methods include but are not limited to isolation and culture of primary T cell sub-populations such as CD3+, CD28+, CD4+, CD8+, and γδ, as well as the isolation and culture of other primary lymphocyte populations such as NK T cells or invariant T cells. Such selection methods can comprise positive and/or negative selection techniques, e.g. wherein the sample is incubated with specific combinations of antibodies and/or cytokines to select for the desired subpopulation. The skilled person can readily adjust the components of the selection medium and/or method and length of the selection using well known methods in the art. Longer incubation times may be used to isolate desired populations in any situation where there is or are expected to be fewer desired cells relative to other cell types, e.g. such as in isolating tumor infiltrating lymphocytes (TIL) from tumor tissue or from immunocompromised individuals. The skilled person will also recognize that multiple rounds of selection can be used in the disclosed methods.

Enrichment of the desired population is also possible by negative selection, e.g. achieved with a combination of antibodies directed to surface markers unique to the negatively selected cells. In a non-limiting example, cell sorting and/or selection via negative magnetic immunoadherence or flow cytometry that uses a cocktail of monoclonal antibodies directed to cell surface markers present on the cells negatively selected can be used. For example, to enrich for CD4+ T cells by negative selection, a monoclonal antibody cocktail typically including antibodies specific for CD14, CD20, CD11b, CD16, HLA-DR, and CD8 is used. The methods disclosed herein also encompass removing T regulatory cells, e.g. CD25+ T cells, from the population to be genetically engineered. Such methods include using an anti-CD25 antibody, or fragment thereof, or a CD25-binding ligand, such as IL-2.

The (genetically engineered) cell (e.g. lymphocyte) of the invention may be a (genetically engineered) autologous cell (e.g. (primary) lymphocyte). The term “autologous” refers to any material isolated, derived and/or obtained from the same individual to whom it is later to be re-introduced, e.g. in the context of an autologous adoptive (immune) cell therapy, such as autologous adoptive T cell therapy (ACT) wherein the same individual is both the donor and recipient. In the context of an autologous cell/transplant, (stem) cells may collected from the patient, may be stored, for example frozen (in liquid nitrogen), and/or may be subjected to transplant conditioning. The (stem) cells may be intrinsically normal, and may, for example, be collected/used for the purpose of allowing blood cell recovery, for example after the administration of a chemotherapy (for example at high doses that would irreversably damage/kill the (stem) cells which remain in the patients body during the chemotherapy). The patient's (stem) cells, may, for example, following transplant conditioning, returned to the patient's body and, for example, (help to) produce healthy (immune) cells, like, for example, blood cells (e.g. red blood cells, white blood cells and/or platelets). Accordingly, in the context of the invention disclosed herein, the genetically engineered cell (e.g. lymphocyte) may be a (genetically engineered) autologous cell (e.g. (primary) lymphocyte), including but not limited to a genetically engineered (primary) autologous NK cell or a (primary) autologous T cell, such as a (primary) autologous CD8+ T cell, a (primary) autologous CD4+ T cell, a (primary) autologous γδ T cell, a (primary) autologous invariant T cell or a (primary) autologous NK T cell. However, the methods and materials disclosed herein (e.g. the genetically engineered lymphocyte) are not limited to autologous cells (e.g. lymphocytes) isolated and/or derived from the subject to be subsequently treated with the cell (e.g. lymphocyte) (and/or to the use of). The methods disclosed herein also encompass the use and production of genetically engineered allogeneic cells (e.g. (primary) lymphocytes). As appreciated in the art, an “allogenic cell” (e.g. “allogeneic lymphocyte”) is a cell (e.g. lymphocyte (e.g. a T cell)) isolated from a donor of the same species as the recipient but not genetically identical to the recipient. It is envisaged in this context that the human leucocyte antigens (HLA) of the donor are acceptable matches to the patient's HLA. The (stem) cell donor may be related to the patient, or may be an unrelated volunteer, found through a donor registry search (such as the National Marrow Donor Program). Allogenic cells can be used in (adoptive) therapies without or, preferably, with further modification, e.g. to reduce or inactivate the allogenic reactions in the intended recipient by the engineered cell (e.g. T cell) to the host (e.g., graft versus host reactions) as well as those immune reactions of the host against the engineered cell (e.g. T cell) (e.g. host versus graft reactions). Such modifications can be made by any method known in the art and/or described herein (such cells are known in the art and referenced herein as “non-alloreactive” or “off-the-shelf” (T-)cells).

The donor and/or recipient of the cells (e.g. lymphocytes) as disclosed herein, including the subject to be treated with the allogenic or autologous genetically engineered cells (e.g. (primary) lymphocytes), may be any living organism in which an immune response can be elicited (e.g. mammals). Examples of donors and/or recipients as used herein include humans, dogs, cats, mice, rats, monkeys and apes, as well as transgenic species thereof, and are preferably humans.

Also provided herein is a method for the production of a (genetically) engineered cell, e.g. lymphocyte (e.g. a human (primary) T cell)) expressing the resistant/less susceptible target and/or receptor as described herein, or (a) functional variant(s) thereof. This method may comprise the step(s) of modifying (e.g. transducing) the cell to express the target and/or receptor, or (a) functional variant(s) thereof, culturing the modified cell under conditions allowing the expression of the target and/or receptor, or (a) functional variant(s) thereof, and recovering said (genetically) engineered cell.

The (genetically) engineered cells (e.g. lymphocytes) of the invention are preferably cultured under controlled conditions, outside of their natural environment. In particular, the term “culturing” as used herein indicates that the engineered cells are maintained in vitro. The (genetically) engineered cells (e.g. lymphocytes) are cultured under conditions allowing the expression of the target and/or receptor, or its functional variant(s). Conditions that allow the maintenance of cells (e.g. lymphocytes) and expression of a desired transgene therein are commonly known in the art and include, but are not limited to culture in the presence of agonistic anti-CD3- and anti-CD28 antibodies, as well as one or more cytokines such as interleukin 2 (IL-2), interleukin 7 (IL-7), interleukin 12 (IL-12) and/or interleukin 15 (IL-15). After expression of the target and/or receptor or (a) functional variant(s)/fragment(s) thereof, as described herein, the (genetically) engineered cell is recovered or otherwise isolated from the culture.

The cells (e.g. lymphocytes) as described herein may be activated and/or expanded as is known in the art. Thus, methods according to the invention may also include a step of activating and/or expanding a (primary) cell (e.g. lymphocyte) or cell (e.g. lymphocyte) population. This can be done prior to or after genetic engineering of the cells, using the methods well known in the art, e.g. as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. As appreciated in the art, such methods can encompass culturing the cells with appropriate agents such as agents that activate stimulatory receptors (e.g. agonistic antibodies) and/or target ligands of endogenous or recombinant receptors as routine in the art. Said cells can also be expanded by co-culturing with tissue or cells expressing target ligands of endogenous or recombinant receptors, including in vivo, for example in the subject's blood after administrating said cells to the subject.

A cell, e.g. an immune cell or progenitor cell thereof, according to the invention, is envisaged to comprise at least one (e.g. also two or more, three or more) target(s) of a DDIA which is (are) resistant against the DDIA or has (have) reduced susceptibility to the DDIA. For example, the cell and/or the target may carry a mutation, or result from a mutation or allele, or (from) two or more mutations or alleles (e.g. 3, 4, 5 or even more), which renders/render said target as being resistant against said DDIA or as having reduced susceptibility to said DDIA. Such a cell, and/or the target, may be made resistant against/less susceptible to the DDIA as described herein elsewhere.

In the cell of the present invention, the resistant/less susceptible target of a DDIA may be introduced in addition to the (native/wildtype) non-resistant/non-less susceptible target or may replace the same. It is, in general, envisaged that the resistant/less susceptible target exhibits the same function(s) as the (native/wildtype, e.g. non-mutated) target, in particular with respect to the avoidance/resolution of TRCs and inimpaired transcription/replication, respectively, even in presence of the DDIA.

The resistance or reduced susceptibility against a DDIA may be a conditional resistance and reduced susceptibility, respectively. “Conditional” resistance/reduced susceptibility in accordance with the invention means that the resistance/reduced susceptibility (e.g. abundance and/or activity of the respective target with resistance/reduced susceptibility against a DDIA) can be controlled (switched off or reduced (and switched on or increased)). The resistance/reduced susceptibility may be conditional to the presence of an agent (see below for examples). In other words, the resistance/reduced susceptibility may be inducible by an agent. In yet other words, the resistance/reduced susceptibility may be triggered by the application of, and respectively may occur only in the presence of, an agent. Further, the (induced) conditional resistance/reduced susceptibility may be restricted or switched off by removing a respective agent. In other words, the (triggered) resistance/reduced susceptibility may be restricted or switched off by terminating the application of, and respectively may no longer occur (or to a lower degree) in the absence of, a respective agent.

Controlling (in particular restricting or switching off) the resistance/reduced susceptibility in a cell, e.g. an immune cell or progenitor cell thereof, may be advantageous under certain situations. For example, this controlling addresses the recurrent need in (conventional) immune cell therapies (e.g. CAR T cell therapies) to terminate the function/proliferation of the immune cells (e.g. CAR T-cells), for example because a (life threatening) overreaction occurs. Especially in such a situation, the resistance/reduced susceptibility, and thus function/proliferation (in the presences of (a) DDIA(s)) can be switched off/reduced.

Means and methods for controlling the expression/abundancy of a protein (e.g. (resistant) target according to the invention) and of conditionally expressing a given function (e.g. resistance/reduced susceptibility according to the invention) are known in the art (e.g. Yesbolatova, NATURE COMMUNICATIONS 11, 2020, 5701; Lawlor, Cancer Res (9), 2006, 5491-601; Nabet, NATURE CHEMICAL BIOLOGY 14, 2018, 431-41).

For example, the resistance and reduced susceptibility, respectively, may be conditional to an FKB analogue (e.g. FKBP1; a FKBP1-dependent regulation system is, for example, described in Nabet loc.cit.), to auxin or an auxin derivative (e.g. Yesbolatova loc.cit.) or to a steroid hormone (e.g. estrogen; an estrogen-dependent regulation is, for example, described in Lawlor loc.cit.). Further, the resistance and reduced susceptibility, respectively, may be conditional to doxycycline (Stieger, Advanced drug delivery reviews 61, 2009, 527-41).

In the context of one particular aspect of the invention, (a) cardiac glycoside(s) (CG(s)) may be used in the context of the herein described cancer/tumor treatments (in particular the chemotherapy or the hybrid treatment comprising the chemotherapy). (A) CG(s) may, for example, be used in place of (a) herein described DDIA(s) or agent(s) which prevent(s) resolution of TRCs (or which provoke(s) TRCs).

The invention further relates to a cell, in particular to an immune cell, or a progenitor cell thereof, which is resistant against a CG, or which exhibits reduced susceptibility to a CG. What is said herein elsewhere with respect to the (immune)cell of the invention, in particular with respect to the (immune)cell of the invention which is resistant against a DDIA or which exhibits reduced susceptibility to a DDIA, and with respect to the respective pharmaceutical compositions, uses, kits, methods of generating, methods of screening, (cell, tissue, organoid, animal) models etc., also applies to the (immune)cell, in particular to an immune cell, or a progenitor cell thereof, which is resistant against a CG, and to the respective pharmaceutical compositions, uses, kits, methods of generating, methods of screening, (cell, tissue, organoid, animal) models etc, mutatis mutandis.

The technical meaning of “CG” is well known in the art and the term “CG” is accordingly used herein. In particular, CGs are chemically characterized by containing (a) deoxy sugar residue(s) glycosidically linked to (a) steroid derivative(s) (or to (a) derivative(s) of gonane). Mechanistically, CGs A) inhibit the sodium-potassium ATPase (Na⁺/K⁺-ATPase) in the cell membrane; and(/or) B) affect relevant (downstream) intracellular pathways, such as the translation of the MYC oncoprotein. A CG to be used in accordance with the invention may prevent/reduce the translation of MYC. This, in turn, may prevent/reduce resolving TRCs in tumor cells. Examples of CGs are known in the art and these are, in principle, envisaged to be used in accordance of the invention. Particular, non-limiting examples of CGs that may be used in accordance of the invention are Cymarin, Coumarin, Ouabain, Digitoxin, Digoxin, Acetyldigitoxin, and Deslanoside.

Cells, in particular immune cells, or progenitor cells thereof, which are resistant against CGs, or which exhibit reduced susceptibility to CGs, can readily be provided/generated by relying on the present disclosure and on means and methods well known in the art and/or as described herein elsewhere and in the appended examples. What is said herein elsewhere with respect to the means and methods for generating a DDIA-resistent target/cell, e.g. an immune cell or progenitor cell thereof, also applies here, mutatuis mutandis. In particular CG-resistant targets may be provided/generated for the purpose of providing/generating CG-resistant cells; for example a CG-resistant Na⁺/K⁺-ATPase. Mutation technologies may be used in this respect, like the CRISPR-Cas technology. Respective means and methods are, for example, described in Neggers (Nat Commun 9(1), 2018 502). For example, cells with (a) mutagenized gene(s)/allele(s) of a target of a CG may be generated (e.g. by using CRISPR-Cas). Further, resistance alleles of Na⁺/K⁺-ATPase are known (e.g. Hiyoshi, Br J Cancer 106(11), 2012, 1807-15). In particular, it is known that the mouse allele of Na⁺/K⁺-ATPase is resistant to CG inhibitors.

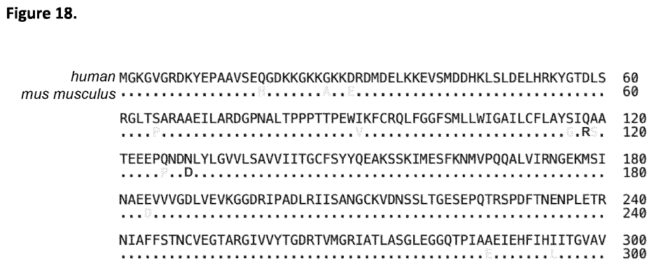

For example, due to a minor difference in the amino acid sequence (see, for example, FIG. 18), the affinity of murine Na⁺/K⁺-ATPase (SEQ ID NO.2) for CGs is approximately 1000-fold lower than that of human Na⁺/K⁺-ATPase (SEQ ID NO.1). A Na⁺/K⁺-ATPase (like the human Na⁺/K⁺-ATPase) may be a target to be present in the cancer/tumor cells to be treated and/or a CG-resistent Na⁺/K⁺-ATPase (like the murine Na⁺/K⁺-ATPase) may be present in the (immune)cell, or in the progenitor cell thereof, which is resistant against a CG, or which exhibits reduced susceptibility to a CG. A CG-resistent Na⁺/K⁺-ATPase may be the murine Na⁺/K⁺-ATPase (SEQ ID NO.2) as such, or a Na⁺/K⁺-ATPase of another origin (e.g. rat or, preferably, human) but with the relevant CG-resistency-conferring difference(s) in the amino acid sequence (cf., for example, FIG. 18).

For example, CG-resistency-conferring (mutations at) amino acid positions are positions 118 and(/or) 129, relative to the murine and/or human Na⁺/K⁺-ATPase (SEQ ID NOs.2 and 1, respectively), or (mutations at) homologous positions in a Na⁺/K⁺-ATPase of another origin. Particular CG-resistency-conferring amino acid residues are a glutamine (R) at position 118 and(/or) an asparagine (D) at position 129, relative to the murine or human Na⁺/K⁺-ATPase (SEQ ID NOs.2 and 1, respectively), or a R and(/or) a D at homologous positions in a Na⁺/K⁺-ATPase of another origin.

A CG-resistent Na⁺/K⁺-ATPase may be Na⁺/K⁺-ATPase with (a) CG-resistancy-conferring mutation(s), e.g. at positions 118 and(/or) 129 relative to the murine or human Na⁺/K⁺-ATPase (SEQ ID NOs.2 and 1, respectively), or at (a) homologous position(s) in a Na⁺/K⁺-ATPase of another origin. A preferred particular example of a CG-resistent Na⁺/K⁺-ATPase is the human Na⁺/K⁺-ATPase (SEQ ID NOs. 1) with (a) CG-resistancy-conferring mutation(s) at positions 118 and(/or) 129, in particular with a Q→R mutation at position 118 and(/or) a N→D mutation at position 129 (cf. FIG. 18). This preferred particular example of a CG-resistent Na⁺/K⁺-ATPase (and any other CG-resistent Na⁺/K⁺-ATPase disclosed herein) may further share one or more of the other amino acid differences which correspond to the amino acid differences of the murine Na⁺/K⁺-ATPase (SEQ ID NOs. 2) as compared to the human Na⁺/K⁺-ATPase (SEQ ID NOs. 1); e.g. 1, 2, 3, 4, 5, 6, 7, 8, 9, 10 or 11 of the Q→H, G→A, D→E, S→P, I→V, S→G, A→S, Q→P, E→D, A→E, and I→L amino acid differences (cf. FIG. 18).

In the (immune)cell, or in the progenitor cell thereof, a CG-resistent Na⁺/K⁺-ATPase may (ectopically) be expressed, or the endogenous Na⁺/K⁺-ATPase may be mutated (e.g. by the CRISPR/Cas9 mutation technology), so as to achieve CG resistancy.

When reference is made herein to Na⁺/K⁺-ATPase, the Na⁺/K⁺-ATPase subunit alpha-1 is particularly meant.

In one aspect, the present invention relates to a pharmaceutical composition comprising (as the/an active ingredient) the (immune) cell (and/or a progenitor cell thereof) according to the invention, i.e. an (immune) cell (and/or progenitor cell thereof) which is resistant against a DDIA or which exhibits reduced susceptibility to a DDIA.

In accordance with this aspect, the pharmaceutical composition may comprise only an (immune) cell (preferred), or only a progenitor cell thereof, of the invention (as the active ingredient). The pharmaceutical composition may, however, also comprise both, the (immune) cell and the progenitor cell thereof according to the invention, or the (immune) cell and another cell described herein. The progenitor cell thereof may, for example, be a (hematopoietic) stem cell. The (immune) cell may be a T-cell (or another lymphocyte), in particular a CAR T-cell. The (hematopoietic) stem cell(s), but likewise also the (immune) cell, may be in form of/provided by a transplant, e.g. cell transplant, e.g. stem cell transplant. The transplant may be an autologous (stem cell) transplant or from an allogenic (stem cell) transplant (see above).

In one aspect, the present invention relates to a pharmaceutical composition, a kit or a combination (set of at least two (or three) components) comprising (e.g. in at least two (or three) different vials)
(i) an (immune) cell and/or a progenitor cell thereof according to the invention (first (and second) component); and
(ii) a DDIA (second (or third) component).

Advantageously, the herein described kit further comprises optionally (a) reaction buffer(s), storage solutions (i.e., preservatives), wash solutions and/or remaining reagents or materials required for the performance of the methods disclosed herein. Parts of the kit of the invention can be packaged individually in vials or bottles or, for example, in combination in containers or multicontainer units.

What has been said with respect to the (immune) cell/progenitor cell above, also applies here mutatis mutandis. For example, one of the components (e.g. comprised in one vial) may be an (immune) cell or a progenitor cell thereof of the invention. A further of these components may be a DDIA (e.g. comprised in another vial), in particular a DDIA against which the (immune) cell/progenitor cell, is resistant or has reduced susceptibility. A further component (e.g. in a third vial) may be another cell, e.g. a (hematopoietic) (stem) cell with resistancy/reduced susceptibility with respect to said DDIA.

A pharmaceutical composition, kit or combination comprising the (immune) cell (or progenitor cell thereof) according to the invention, the (hematopoietic) stem cell according to the invention (or another cell of the invention), or both, can, for example, advantageously be used in a chemotherapeutic treatment of (solid) tumors/cancers by using a DDIA (or two or more DDIAs). For example, in such a treatment, the DDIA(s) target(s) the tumor/cancer (e.g. by introducing TCRs or by targeting resolution of TCRs in respective tumor/cancer cells), the immune cell (or progenitor cell thereof) targets the tumor/cancer cells immunologically, e.g. in the context of an (adoptive) immune cell therapy (for example through binding of a CAR to (a) TSA(s) or TAA(s). In addition, the (hematopoietic) stem cells (or other cells) may replace/supplement other (endogenous/native) cells which undesirably may also be impaired by the used DDIA(s), like, for example, (endogenous/native) hematopoietic stem cells). The negative impact of the used DDIA(s) can thus be further reduced or compensated by the third component, e.g. the (hematopoietic) stem cell (or another cell).

As also evident from this description and from the appended examples, the means and methods provided herein, in particular the (immune) cell (or progenitor cell) of the invention, are particularly useful and may be used in the treatment of tumors/cancers, more particular in the treatment of tumors, even more particular in the treatment of solid tumors and in the treatment of (solid) tumors/cancers with large and currently unmet clinical needs (“undruggable” (solid) tumors/cancers in other words). Thus, in one aspect, the present invention relates to a pharmaceutical, kit or combination according to the invention; or to (i) an (immune) cell and/or a progenitor cell thereof according to the invention; and (ii) a DDIA, in particular a DDIA as described herein, for use in treating a cancer and/or a tumor (i.e. the respective cancer/tumor cells), more particular a tumor, even more particular a solid tumor; or for use in treating a proliferative disease.

In general, any tumor or cancer may be envisaged to be treated in accordance with the invention. “Cancer” is especially meant to refer to malignant cancers, including malignant tumors. “Tumor” is especially meant to refer to solid tumors, including malignant and non-malignant tumors. The meaning of “cancer” in accordance with the invention also comprises solid cancers/tumors. It is particularly envisaged that malignant tumors/cancers are to be treated in accordance with the invention. The treatment of malignant solid tumors is even more preferred. The terms “cancer” and “tumor” are used accordingly in the context of the invention. Thus, when reference is made to “tumor” in the context of the invention, solid tumors are particularly meant, more particular, malignant solid tumors are meant. However, the treatment of non-malignant tumors and unsolid tumors is not necessarily excluded. Although, in general, any tumor/cancer, may be treated in accordance with the invention, the treatment of un-solid cancers/tumors, e.g. cancers/tumors of the blood (hematoma), is less preferred.

The term “cancer” or “proliferative disease” as used herein means any disease, condition, trait, genotype or phenotype characterized by unregulated cell growth and/or replication as is known in the art.

In a preferred embodiment, the means and methods provided herein, in particular the (immune) cells (or progenitors thereof) of the invention, or the pharmaceutical composition, kit, combination or (combination of) immune cell and/or progenitor cell thereof and DDIA of the invention, or the hybrid therapy according to the invention, are to be used in the treatment of solid tumors. As mentioned, the solid tumors, and also other tumors/cancers to be treated in accordance with the invention, may be tumors/cancers with large and currently unmet clinical needs or “undruggable” tumors/cancers.

The tumor/cancer to be treated in accordance with the invention, may be a malignant and/or metastasizing tumor tumor/cancer. As described herein and in the appended examples, the means and methods of the present invention are particularly useful for, and may be employed in, the treatment of malignant and/or metastasizing solid tumors.

The present invention further relates to the treatment of metastases and/or to the reduction or prevention of (the formation of) the same, and/or to the reduction or prevention of the growth of the same, and/or to the treatment (including prevention) of tumors/cancers which result in the formation of such metastases. The means and methods provided herein, in particular the (immune) cells (or progenitors thereof) of the invention (or the pharmaceutical composition, kit, combination or (combination of) immune cell and/or progenitor cell thereof and DDIA of the invention), are thus to be used in the treatment (including prevention) of (the formation of) metastases and/or to the reduction or prevention of the same, and/or to the reduction or prevention of the growth of the same, and/or to the treatment (including prevention) of tumors/cancers which result in the formation of such metastases.

The present invention further relates to the means and methods provided herein, in particular the (immune) cell (or progenitor cell), the pharmaceutical composition, kit or combination of the invention, for use in the regression of a tumor/cancer.

The present invention further relates to the means and methods provided herein, in particular the (immune) cell (or progenitor cell), the pharmaceutical composition, kit or combination of invention, more particular the DDIA described and disclosed herein (e.g. the DDIA to be screened as described herein), for use in sensitizing cells of a tumor/cancer to an (adoptive) immune cell therapy, e.g. to a therapy with an (immune) cell of the invention. Thus, the present invention also relates to an (immune) cell therapy including the use of (a) DDIA(s) for sensitizing cells of a tumor/cancer to the (immune) cell therapy.

Further, (immune) cell-mediated killing of cells of a tumor/cancer, in particular by an (immune) cell of the invention, may be performed in accordance with the invention (separately and independently from, or in combination with, any of the other treatment methods described herein, e.g. chemotherapy with (a) DDIA(s)).

Further, escape from (immune) cell-mediated killing/surveillance/regression (immune) of (cells of) a tumor/cancer, in particular escape from an (immune) cell of the invention, may be prevented in accordance with the invention (separately and independently from, or in combination with, any of the other treatment methods described herein, e.g. chemotherapy with (a) DDIA(s)).

The present invention further relates to the means and methods provided herein, in particular the immune cell (or progenitor cell), the pharmaceutical composition, kit or combination of invention, more particular the DDIA described and disclosed herein (e.g. the DDIA to be screened as described herein), for use in controlling an (immune) cell therapy. It is particularly envisaged that said controlling and/or said (immune) cell therapy comprises the use of an (immune) cell or progenitor cell thereof according to the invention. In this context, the conditional resistance or conditional reduced susceptibility as described herein above may be employed.

The present invention further relates to the means and methods provided herein, in particular the (immune) cell (or progenitor cell), the pharmaceutical composition, kit or combination of invention, for use in mimicking an intact immune system (separately and independently from, or in combination with, any of the other treatment methods described herein, e.g. chemotherapy with (a) DDIA(s)). Mimicking an intact immune system is considered to be particularly useful in an immune deficient patient. Mimicking an intact immune system can be achieved by administering the (immune) cells of the invention. In particular, the mimicking of an intact immune system for the (cells of) a tumor/cancer as described herein is envisaged. This means that the administered (immune) cells of the invention serve like cells of the immune system and, as such, target said (cells of) a tumor/cancer (for example in cases where the deficient immune system itself fails to do so).

A tumor/cancer to be treated in accordance with the invention may, in principle, be any tumor/cancer. However, the mechanisms, means and methods described herein are considered to be particularly advantageous for MYC/MYCN-driven tumors/cancers. For example, (c-)MYC (herein also MYC) binds only very weakly to Aurora-A kinase (Dauch, Nat Med 22, 2016, 744-753). Thus, especially in this context, a DDIA-resistant/less susceptible variant of Aurora-A kinase (e.g. the T217D or T217E mutant) is particularly useful to be comprised in the (immune) cell/progenitor of the invention. Thus, it is particularly envisaged in the context of the invention to treat MYC/MYCN-driven tumors/cancers. This in fact encompasses the majority of all tumors/cancers, in particular the vast majority of all tumors.

In the context of the invention, A) targets (e.g. Na⁺/K⁺-ATPase) have been identified (or may be identified) that lead to a reduction of MYC in the cancer/tumor and(/or) to a potential visibility of the tumor to the immune system; and(/or) B) (immune) cells, or progenitor cells thereof, are modified so that they are independent of the identified target, for example, in their function and expansion potential. As mentioned, such (immune) cells may be provided/generated by providing/generating a respective (DDIA-/CG-)resistant target.

In one embodiment, the cancer/tumor to be treated in accordance with the invention is a C-Myc-, L-Myc- and/or N-Myc-driven cancer/tumor. Respective cancers/tumours are known in the art and are, for example, colon, lung and pancreas cancers/tumors.

In one embodiment, the cancer/tumor to be treated in accordance with the invention is a cancer/tumor which expresses one or more tumor marker(s) as described herein, e.g. in Table 1. Evidence exists that also the tumors/cancers as refereed to in Table 1 are MYC/MYCN-driven tumors/cancers. Thus, the mechanisms, means and methods described herein are considered to be particularly advantageous also for these particular cancers.

In general, non-limiting examples of cancers and/or tumors to be treated in accordance with the invention are selected from the group consisting of colorectal tumors/cancers, brain tumors/cancers, ovarian tumors/cancers, prostate tumors/cancers, pancreatic tumors/cancers, breast tumors/cancers, renal tumors/cancers, nasopharyngeal carcinoma, hepatocellular carcinoma, melanoma, skin tumors/cancers, oral tumors/cancers, head and neck tumors/cancers, esophageal tumors/cancers, gastric tumors/cancers, cervical tumors/cancers, bladder tumors/cancers, lymphoma, chronic or acute leukemia (such as B, T, and myeloid derived), sarcoma, lung tumors/cancers and multidrug resistant tumors/cancers. This includes the treatment (and prevention) of metastases which derive from any of the above-mentioned tumors/cancers. Examples of particular solid tumors to be treated are selected from the group consisting of PDAC, CRC and (pediatric) neuroblastoma.

Further, particular but non-limiting examples of cancers/tumors to be treated in accordance with the invention are selected from the group consisting of
(i) pancreas cancers/carcinomas and/or tumors, in particular PDAC;
(ii) colon cancers/carcinomas and/or tumors, in particular (metastatic) CRC, and/or resulting metastases (e.g. in the liver);
(iii) (pediatric) neuroblastoma;
(iv) udenocarcinoma;
(v) glioblastoma; or
(vi) melanoma.

The present invention further relates to a pharmaceutical composition comprising (and the herein-described pharmaceutical compositions may further comprise) a DDIA as defined and described herein, or a (novel) DDIA to be screened in accordance with the respective screening methods of the invention. In one aspect, also this pharmaceutical composition may be for use in treating a cancer/tumor as defined and described herein. In this context, it is particularly envisaged that the cancer/tumor is PDAC or CRC and/or metastases resulting therefrom.

The terms “treatment”, “treating” and the like are used herein to generally mean obtaining a desired pharmacological and/or physiological effect. The effect may be prophylactic in terms of completely or partially preventing a disease or symptom thereof, and/or may be therapeutic in terms of partially or completely curing the disease or condition, and/or adverse effect attributed to the disease or condition. The term “treatment” as used herein covers any treatment of a disease or condition in a subject and includes: (a) preventing and/or ameliorating a proliferative disease (preferably tumor/cancer) from occurring in a subject that may be predisposed to the disease; (b) inhibiting the disease, i.e., arresting its development, such as inhibition of cancer progression; (c) relieving the disease, i.e. causing regression of the disease, such as the repression of cancer; and/or (d) preventing, inhibiting or relieving any symptom or adverse effect associated with the disease or condition. Preferably, the term “treatment” as used herein relates to medical intervention of an already manifested disorder, e.g. the treatment of a diagnosed tumor/cancer.

The treatment or therapy (i.e., comprising the use of a medicament/pharmaceutical composition comprising a genetically engineered cell (e.g. lymphocyte) as disclosed herein) may be administered alone or in combination with appropriate treatment protocols for the particular disease or condition as known in the art. Non-limiting examples of such protocols include but are not limited to, administration of pain medications, administration of chemotherapeutics (DDIAs; preferred), therapeutic radiation, and surgical handling of the disease, condition or symptom thereof. Accordingly the treatment regimens disclosed herein encompass the administration of the genetically engineered cell (e.g. lymphocyte) expressing resistant target and/or receptor, or (a) functional variant(s) thereof, together with none, one, or more than one treatment protocol suitable for the treatment or prevention of a disease, condition or a symptom thereof, either as described herein or as known in the art.

Administration “in combination” or the use “together” with other known therapies encompasses the administration of the medicament/pharmaceutical composition comprising a genetically engineered cell (e.g. lymphocyte) as disclosed herein before, during, after or concurrently with any of the co-therapies disclosed herein or known in the art. The genetically engineered cells (e.g. lymphocytes) disclosed herein (or the pharmaceutical composition/medicament comprising such cells (e.g. lymphocytes) can be administered alone or in combination with other therapies or treatments during periods of active disease, or during a period of remission or less active disease.

When administered in combination, the (genetically engineered) (immune) cell (e.g. lymphocyte) immunotherapy (e.g. adoptive (T) cell therapy, ACT) and/or any additional therapy, can be administered in an amount or dose that is higher, lower or the same than the amount or dosage where each therapy or agent would be used individually, e.g. as a monotherapy. In certain embodiments, the administered amount or dosage of the (genetically engineered) (immune) cell (lymphocyte) therapy, and/or at least one additional agent or therapy is lower (e.g. at least 20%, at least 30%, at least 40%, or at least 50%) than the amount or dosage of the corresponding therapy(ies) or agent(s) used individually.

The (genetically engineered) cell of the invention may have been recombinantly modified ex vivo to express the resistant target and/or receptor, or (a) functional variant(s) thereof. Alternately or additionally, the (genetically engineered) cell (e.g. lymphocyte) may be pulsed with (a) tumor antigen(s), for example prior to modification with the respective nucleic acid molecule(s).

The (genetically engineered) cells (e.g. lymphocytes) of the invention may undergo robust in vivo (T cell) expansion upon administration to a patient, and may remain persist in the body fluids for an extended amount of time, preferably for a week, more preferably for 2 weeks, even more preferably for at least one month. Although the (genetically engineered) cells (e.g. lymphocytes) according to the invention are expected to persist during these periods, their functional life span is envisaged to be in an appropriate range. For example, it is not expected to exceed more than a year, no more than 6 months, no more than 2 months, or no more than one month. The cells of the invention may also be additionally engineered with safety switches that allow for potential control of the cell therapeutics (see also above). Such safety switches of potential use in cell therapies are known in the art and include (but are not limited to) the engineering of the cells to express targets allowing antibody depletion (e.g. truncated EGFR; Paszkiewicz, J Clin Invest 126(2016), 4262-4272), introduction of artificial targets for small molecule inhibitors (e.g. HSV-TK; Liang, Nature 563(2018), 701-704) and introduction of inducible cell death genes (e.g. icaspase; Minagawa, Methods Mol Biol 1895(2019), 57-73).

The administration of the cells (e.g. lymphocytes) or population of cells (e.g. of lymphocytes) according to the present invention may be carried out in any convenient manner, including by aerosol inhalation injection, ingestion transfusion, implantation or transplantation. The medicaments and compositions described herein may be administered subcutaneously, intradermally, intratumorally, intranodally, intramedullary, intramuscularly, by intravenous or intralymphatic injection, or intraperitoneally. The cells (e.g. lymphocytes), medicament and/or compositions of the present invention are preferably administered by intravenous injection.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. For example, the genetically engineered cells (e.g. lymphocytes) of the invention may be administered to the subject at a dose of 10⁴ to 10¹⁰ (T) cells/kg body weight, preferably 10⁵ to 10⁶ (T) cells/kg body weight. In the context of the present invention the cells (e.g. lymphocytes) may be administered in such a way that an upscaling of the (T) cells to be administered is performed by starting with a subject dose of about 10⁵ to 10⁶ (T) cells/kg body weight and then increasing to dose of 10¹⁰ (T) cells/kg body weight. The cells or population of cells can be administrated in one or more doses.

The term “medicament” is used interchangeably with the term “pharmaceutical composition” and relates to a composition suitable for administration to a patient, preferably a human patient. The medicament/pharmaceutical composition may be administered to an allogenic recipient, i.e. to recipient that is a different individual from that donating the (T) cells, or to an autologous recipient, i.e. wherein the recipient patient also donated the (T) cells. Alternately the medicament/pharmaceutical composition may comprise non-allogenic cells (e.g. lymphocytes), (“off the shelf” cells (e.g. lymphocytes) as known in the art). Regardless of the species of the patient, the donor and recipient (patient) are of the same species. It is preferred that the patient/recipient is a human.

In the manufacture of a pharmaceutical formulation according to the invention, the (genetically engineered) cells (e.g. lymphocytes) are typically admixed with a pharmaceutically acceptable carrier excipient and/or diluent and the resulting composition is administered to a subject. The carrier must, of course, be acceptable in the sense of being compatible with any other ingredients in the formulation and must not be deleterious to the subject or (engineered) cells. Examples of suitable pharmaceutical carriers are well known in the art and include phosphate buffered saline solutions, water, emulsions, such as oil/water emulsions, various types of wetting agents, sterile solutions etc. The carrier may be a solution that is isotonic with the blood of the recipient. Compositions comprising such carriers can be formulated by well known conventional methods. The pharmaceutical compositions of the invention can further comprise one or more additional agents useful in the treatment of a disease in the subject. Where the (genetically-modified) cell (e.g. lymphocyte) is a (primary) human T cell (or a cell derived therefrom), pharmaceutical compositions of the invention can further include biological molecules, such as cytokines (e.g., IL-2, IL-7, IL-15, and/or IL-21), which promote in vivo cell proliferation and engraftment. The (genetically modified) cells (e.g. lymphocytes) of the invention can be administered in the same composition as the one or more additional agent or biological molecule or, alternatively, can be co-administered in separate compositions/containers.

In addition, the kit may contain instructions for use. The manufacture of the described kit preferably follows standard procedures, which are known to the person skilled in the art. Further, any of the pharmaceutical compositions, cells, active ingredients, combinations, kits etc. of the invention may be provided together with an instruction manual or instruction leaflet for use. The instruction manual/leaflet may comprise guidance for the skilled person/attending physician how to treat or prevent a disease, disorder or symptom as described herein in accordance with the invention, in particular cancers/tumors and proliferative diseases. In particular, the instruction manual/leaflet may comprise guidance as to the herein described mode of administration/administration regimen (for example route of administration, dosage regimen, time of administration, frequency of administration). In principle, what has been said herein elsewhere with respect to the mode of administration/administration regimen may be comprised in the instruction manual/leaflet. More particular, the instruction manual/leaflet may comprise guidance as to the herein described hybrid tumor/cancer therapy.

In the context of one aspect of the invention, it is envisaged that DNA damage is induced, e.g. in the cancer/tumor cells, by using a proteolysis targeting chimera (PROTAC). In particular, the PROTAC is envisaged to inhibit/deplete (the function of) a respective target (e.g. a target as described herein elsewhere; like a target as described under item 26, infra). Thereby, the resolution of TRCs in cancer/tumor cells may be targeted by a PROTAC; or TRCs in cancer/tumor cells may be induced by a PROTAC.

In accordance with this aspect, also (immune) cells, or progenitors thereof, are provided that are resistant against the PROTAC, or which exhibit reduced susceptibility to the PROTAC. Respective resistant/less susceptible targets and alleles/mutations which confer resistancy against/less susceptibility to a PROTAC when comprised in these cells are known in the art or can be screened/identified with the screening methods described herein.

PROTACs to be used in accordance with the invention may be based on an immunomodulator such as thalidomide or a thalidomide derivative (e.g. lenalidomide or pomalidomide). Lenalidomide or pomalidomide are particular examples of PROTACs which may be used in accordance with this particular aspect of the invention.

An example of a target of a PROTAC (i.e. a target which inhibition/depletion induces DNA damage and/or prevents the resolution of TRCs or induces TRCs in cancer/tumor cells) is cereblon. Accordingly, cereblon with (a) mutation(s) conferring resistancy against/less susceptibility to a PROTAC (e.g. against/to lenalidomide or pomalidomide), and/or (a) respective allele(s), may be comprised in the (immune) cells, or progenitors thereof, of the invention. An example of a respective resistancy-/less susceptibility-conferring allele/mutation is the cereblon V3911 allele/mutation. Further provided is a hybrid therapy which uses a PROTAC for inducing DNA damage (e.g. in the cancer/tumor cells), and (immune) cells, or progenitors thereof, which are resistant against/less susceptible to the PROTAC.

In general, the PROTAC technology is well known in the art and has initially been described by Sakamoto (PNAS 98 (15), 2001: 8554-9). A PROTAC is meant to be a heterobifunctional small molecule composed of two active domains (and a linker). A PROTAC works by inducing selective intracellular proteolysis of the respective target. More particular, PROTACs usually consist of two covalently linked protein-binding molecules: one capable of engaging an E3 ubiquitin ligase, and another that binds to a target (meant for inhibition/depletion). Recruitment of the E3 ligase to the target results in ubiquitination and subsequent inhibition/depletion of the target by the proteasome.

What has been said herein elsewhere with respect to the described means and methods for an improved medical intervention of tumors/cancers also applies to this particular aspect of the invention, mutatis mutandis.

In general, the patient is to be treated in accordance with the invention is envisaged to be any patient in need of the treatment. The patient is to be treated in accordance with the invention may be an immune competent patient or an immune deficient patient. In one particular (non-limiting) aspect, the patient is an immune competent patient.

In general, DDIAs are well known in the art (see, for example, Wang, J Biol Chem 274(31), 1999, 22060-4). The term “DDIA” and its respective technical meaning is accordingly used herein. In particular, a DDIA to be used in accordance with the invention may be a transcription-replication conflict-inducing agent (TRCIA). The meaning of “TRCIA” as used throughout the invention is envisaged to include both, agents which prevent/target resolution of TRCs, or agents which (directly) introduces TRCs. The meaning of TRCs is also known in the art and used accordingly herein. An exemplary outline of the understanding of TRCs as known in the art is given in the following:

Deregulated transcription raises the inherent risk of conflicts with the replication fork ((Garcia-Muse loc.cit.; Hamperl loc.cit.). One major reason for this is that perturbances in transcription lead to the accumulation of R-loops, which are stable hybrids between nascent mRNA and the double-stranded DNA (Crossley loc.cit.). R-loop formation displaces one DNA strand, causing frequent single-strand DNA breaks, and are an impediment to the replication fork, causing collisions between RNA polymerases and the replication fork. If collisions occur, double-strand breaks are caused due to the accumulation of excessive torsional stress between the DNA and RNA polymerase complexes. TRCs are particularly difficult to resolve when RNA polymerase stalls, for example due to low nucleotide concentrations (Noe Gonzalez loc.cit.). Co-directional conflicts may also be employed. Without being bound by theory, these may occur because the replication fork moves faster than RNA polymerases (cf. e.g. Hamperl, loc.cit.). Co-directional conflicts may activate potential targets of DDIAs, e.g. the ATM kinase. Thus, the means and methods provided herein may be even more potent when relying on combined with the inhibition of such potential targets of DDIAs, e.g. inhibitors of ATM kinase; see below for further details.

A DDIA to be used in accordance with the invention may be a small molecule. Multiple examples of small molecule DDIAs are described herein elsewhere (e.g. in item 27, infra).

Any suitable target can, in principle, be used as the target in accordance with the invention, i.e. as the target of a DDIA (like the DDIAs as described and disclosed herein). Being a “target” and “targeting”, respectively, in accordance with the invention means that, once a target is impaired by a DDIA, DNA damage occurs, for example due to/as a result of TCRs. As a result, the proliferation/cell growth is reduced or inhibited. What has been said herein elsewhere with respect to DDIA, DNA damage and TCRs applies here, mutatis mutandis. In one aspect, “targeting” in accordance with the invention means impairing (ongoing) DNA replication (cf., for example, Wang loc.cit.). “Impairing” in this context means that the function (e.g. in DNA replication) is negatively impaired, e.g. (partially) reduced or (entirely) inhibited. That is, a target is impaired once its function (e.g. in DNA replication) is (partially) reduced or (entirely) inhibited, or once the target is depleted (partially or entirely). DNA replication is impaired, once it is (partially) reduced or (entirely) inhibited and/or once TCRs occur. As also mentioned herein elsewhere, a DNA damage may occur in case the resolution of TRCs is impaired (e.g. in ((a) cell(s) of) a tumor/cancer), or in case TRCs are (directly) induced (e.g. in ((a) cell(s) of) a tumor/cancer).

“Reduced”/“reduction” (e.g. “reduced susceptibility”, “reduced function”, “reduced expression”), “partial” (e.g. “partial depletion”), “less” (e.g. “less susceptibility”, “less susceptible”) etc. in the context of the invention means, for example, at most 95%, at most 90%, at most 80%, at most 70%, at most 60%, at most 50%, at most 40%, at most 30%, at most 20%, at most 10%, or at most 5%. This relates to, for example, the full/unimpaired susceptibility, depletion, function, expression, occurrence etc. (e.g. of the target/target expression, DNA replication, receptor/receptor expression etc.). For example, “reduced susceptibility” of a target in accordance with the invention means at least 5%, at least 10%, at least 20%, at least 30%, at least 40%, at least 50%, at least 60%, at least 70%, at least 80%, at least 90% or at least 95 of the unimpaired function of the target (e.g. in unimpaired transcription and/or replication; and/or in resolving TRCs). For example, “resistant/resistancy” with respect to a target in accordance with the invention means unimpaired, or at lease almost inimpaired (e.g. more than 95%), function of the target (e.g. in unimpaired transcription and/or replication; and/or in resolving TRCs).

Means and methods for determining DNA damage, and for determining whether a given compound is a DDIA, are known in the art (e.g. Quah loc.cit.; Barrentina loc.cit.) and are described in the appended Examples (e.g. Example 1 and 7).

Non-limiting examples of targets of DDIAs to be employed in accordance with the invention are
(i) Aurora A kinase;
(ii) Ataxia telangiectasia and Rad3-related (ATR) kinase;
(iii) PAFc complexes;
(iv) PAF1 (e.g. CDC73, LEO1, CTR9);
(v) CDK9;
(vi) CDK12;
(vii) cyclinK/CDK12 complexes;
(viii) splicing factors (e.g. SF3B1, RBM39) and/or transcription termination factors;
(ix) SNRNP70;
(x) CPSF1;
(xi) CPSF3;
(xii) PNUTs/PP11 phosphatase complex;
(xiii) NUAK1/ARK5;
(xiv) RNA polymerase 1 (POL1);
(xv) ATM kinase;
(xvi) Na⁺/K⁺-ATPase, in particular human Na⁺/K⁺-ATPase;
(xvii) USP28;
(xviii) Topoisomerase I;
(xix) Topoisomerase II; and
(xx) Poly(ADP-ribose)-Polymerase.

One particular example of a target in accordance with the invention is the Aurora A Kinase. It has been reported that the inhibition of Aurora A Kinase results in TCRs; and that there are existing Aurora A Kinase mutations/alleles which have been demountrated to confer resistancy against available Aurora A Kinase inhibitors (e.g. T217D and T217E; see Roeschert loc.cit.; Sloane loc. cit).

Another particular example of a target in accordance with the invention is the Na⁺/K⁺-ATPase, in particular the human Na⁺/K⁺-ATPase. It has been reported that the inhibition of Na⁺/K⁺-ATPase causes DNA damage. Further, resistance alleles of this enzyme are known (Hiyoshi, Br J Cancer 106(11), 2012, 1807-15). In particular, it is known that the mouse allele of Na⁺/K⁺-ATPase is resistant to inhibitors, in particular to CGs.

References for the native and resistant versions of the Aurora A Kinase and the Na⁺/K⁺-ATPase can be found in Sloane, ACS Chem Biol 5, 2010 563-576 and Steinberger, Cell Chem Biol 26 2019, 699-710 e696, respectively.

Particular DDIAs which may be used in accordance with the invention are DDIAs which target Myc, i.e. which result in a reduction/depletion of (the expression and/or function of) Myc. DDIAs which can be used as Myc-targeting DDIAs are described herein elsewhere and are known in the art.

Non-limiting examples of particular DDIAs to be employed in accordance with the invention are
(i) Aurora A kinase inhibitors (e.g. MLN8054, MLN8237 (Alisertib; Millennium), LY3295668); it is preferred that (at the relevant doses) the Aurora A kinase inhibitors specifically attack Aurora A (and, for example, not Aurora B));
(ii) ATR kinase inhibitors (e.g. AZD6738 (Astra-Zeneca), BAY 1895344 (Bayer));
(iii) PAFc complex inhibitors or inhibitors of any subunit of the PAFc complex;
(iv) CDK9 inhibitors (e.g. AZD4573, NVP-2, CYC065 (fadraciclib), THAL-SNS-03);
(v) CDK12 inhibitors (e.g. SR4835, THZ-531);
(vi) cyclinK/CDK12 complexes inhibitors (e.g. CR-8);
(vii) splicing and/or termination complexes inhibitors (e.g. insidulam, SPI-21 (Bahat, Mol Cell 76, 2019, 617-31 e614), Pladienolide B, H3B-8800)
(viii) SNRNP70 inhibitors;
(ix) CPSF1 inhibitors;
(x) CPSF3 inhibitors (e.g. JTE-607);
(xi) PNUTs/PP11 phosphatase complex inhibitors (e.g. calyculin A);
(xii) NUAK1/ARK5 inhibitors (e.g. BAY-880 (Bayer), ON-123300, XMD-1571, HTH-01-015);
(xiii) POL1 inhibitors (e.g. CX-5461);
(xiv) ATM kinase inhibitors (e.g. KU-60019, KU-559403, AZD1390);
(xv) Na⁺/K⁺-ATPase inhibitors (e.g. coumarin, ouabain, cymarin, digitoxin, digoxin, acetyldigitoxin, and deslanoside.);
(xvi) USP28 inhibitors (e.g. FT206, AZ1);
(xvii) Topoisomerase I inhibitors (e.g. Irinotecan, topotecan, campthotecin);
(xviii) Topoisomerase II inhibitors (e.g. etoposide, doxorubicin, daunorubicin); and
(xix) Poly(ADP-ribose)-Polymerase inhibitors (e.g. olaparib, veliparib).

In principle, one or more, i.e. at least two, different DDIAs, may be administered in accordance with the invention. For example, 2 or more, 3 or more, 4 or more or 5 or more, 6 or more, 7 or more different DDIAs may be administered. However, most commonly, 1, 2, 3 or 4 different DDIAs may be administered in accordance with the invention. As also shown herein and in the appended examples, 2 different DDIAs may be administered advantageously in accordance with the invention. For example, one of said two (or more) different DDIAs may be an ATR kinase inhibitor (e.g. at a low dose) or an ATM kinase inhibitor (e.g. at a low dose). An ATR kinase inhibitor is preferred to be administered as one of the two (or more) DDIAs.

An example of a particular combination of two DDIAs to be used in accordance with the invention (or to be combined with one or more further DDIA(s)) is a CDK12 inhibitor (e.g. SR4835, THZ-531) and an ATR kinase inhibitor (e.g. AZD6738, BAY 1895344) or an ATM kinase inhibitor (e.g. KU-60019, KU-559403, AZD1390). Another example is a SNRNP70 inhibitor or a CPSF1 inhibitor and an ATR kinase inhibitor (e.g. AZD6738, BAY 1895344). A further example is a NUAK1 inhibitor (e.g. BAY-880, ON-123300, XMD-1571, HTH-01-015) and an ATR kinase inhibitor (e.g. AZD6738, BAY1895344). A further example is an RNA polymerase I inhibitor (e.g. CX-5461) and an ATR kinase inhibitor (e.g. AZD6738, BAY 1895344). Yet a further example is a splicing and/or termination complexe(s) inhibitor (e.g. insidulam, SPI-21, Pladienolide B, H3B-8800) and an ATR kinase inhibitor (e.g. AZD6738, BAY 1895344).

Non-limiting examples of both, at least one particular target of a DDIA with resistance against/reduced susceptibility to said DDIA and said DDIA, are selected from the group consisting of
(i) Aurora A kinase T217E or T217D mutant (or another DDIA-resistant Aurora A kinase mutant) and MLN8054, MLN8237 or LY3295668, respectively (e.g. for use in the treatment of (pediatric) neuroblastoma);
(ii) CDK12 C1039S mutant and THZ-531, respectively (e.g. for use in the treatment of CDK12-dependent tumors, like triple-negative breast cancer/tumor);
(iii) RBM39 G268V mutant and indisulam, respectively (e.g. for use in the treatment of MYC or MYCN-driven tumors, like colon, pancreatic and small cell lung cancers/tumors);
(iv) murine Na⁺/K⁺-ATPase (or another CG-resistant Na⁺/K⁺-ATPase; see herein elsewhere) and coumarin, ouabain, digitoxin, cymarin, digoxin, acetyldigitoxin, deslanoside, or another Na⁺/K⁺-ATPase inhibitor (CG), respectively (e.g. for use in the treatment of MYC-dependent cancers/tumors, like colon and pancreatic cancers/tumors);
(v) topoisomerase I with (a) mutation(s) that confer(s) resistance to (a) topoisomerase I inhibitor(s) and a topoisomerase I inhibitor, respectively, e.g. a topoisomerase I F361S, G363C and/or R364H mutant and campthotecin, respectively; or a topoisomerase I S365G, R621H and/or E710G mutant and irinotecan, respectively (e.g. for use in the treatment of the (solid)cancers/tumors as disclosed and described herein);
(vi) topoisomerase II with (a) mutation(s) that confer(s) resistance to (a) topoisomerase inhibitor(s) and a topoisomerase II inhibitor, respectively, e.g. a topoisomerase II P501, G776 and/or K505 mutant and etoposide, doxorubicin or mitoxantron, respectively; and
(vii) a deletion of the cellular PARP gene and a PARP inhibitor, respectively (e.g. olaparib or veliparib) (e.g. for use in the treatment of the cancers/tumors as disclosed and described herein).

In one aspect, the present invention relates to method of screening for a target of a DDIA which is resistant against said DDIA or has reduced susceptibility to said DDIA. Said method of screening may comprise the steps of:
(a) generating a pool or library of (immune) cells with one or more mutations (e.g. (a) point mutation(s) and/or (small) deletions) in (a) gene(s)/(an) allele(s) of one or more (potential) target(s) of a given DDIA;
(b) contacting said pool or library of (immune) cells with a/said given DDIA;
(c) selecting cells of said pool or library of (immune) cells which are resistant against said DDIA or have reduced susceptibility to said DDIA;
(d) recovering the cells which have been selected according to step (c); or, preferably and
(e) recovering from said cells as recovered according to step (d) (optionally including sequencing) said mutated gene(s)/allele(s) of one or more (potential) target(s) of a given DDIA, thereby identifying said target of a DDIA which is resistant against said DDIA or has reduced susceptibility to said DDIA.

A pool or library of cells with (a) mutagenized gene(s)/allele(s) of a target of a DDIA may be generated (e.g. according to step (a), supra) by using CRISPR-Cas, in particular CRISPR-Cas mutagenesis. Respective means and methods are, for example, described in Neggers (Nat Commun 9(1), 2018 502). Also the identification of a resistant/less susceptible target of a DDIA (e.g. according to step (e), supra) may be performed according to Neggers (loc.cit.)

The above-described method of screening for a resistant/less susceptible target of a DDIA, in particular step (e) thereof, may also be performed by using viral, e.g. lentiviral, libraries and/or by be screening of (lentiviral) libraries, for example as described in Cluse (Methods Mol Biol 1725, 201-27). In this context, (lentiviral) transduction of (immune) cell (e.g. (CAR) T-cells) may also be employed, as, for example described in Prommesberger (loc.cit.). Further, stimulation of (T-) cells, and measurement of their functionality, may be employed, as, for example, described in Reinwald (loc.cit.). Further, (immune) cell proliferation may be measured, as, for example, described in Quah (loc.cit). Moreover, screening of cell lines/cells for drug (DDIA) sensitivity, as, for example, described in Barrentina (loc.cit), may be embloyed.

The method of screening for a target of a DDIA according to the invention may further comprise the step(s) of
(f) reintroducing said mutated gene(s)/allele(s) of one or more (potential) target(s) of a given DDIA as recovered according to step (e), supra, into an (immune) cell (preferably a naïve (immune) cell); and/or
(g) testing/confirming whether said mutated gene(s)/allele(s) of one or more (potential) target(s) of a given DDIA is capable of conferring resistance or reduced susceptibility to said (immune) cell (e.g. by contacting said (immune) cell with said DDIA and assaying the activity of the target encoded by said mutated gene(s)/allele(s)).

Also, in this context (lentiviral) transduction of (T)-cells may be performed, for example as described in Prommersberger (loc.cit). Stimulation of (T)-cells and measurement of their functionality may be performed, for example as described in Reinwald (loc.cit.) and/or measurements of (immune) cell proliferation may be performed, as, for example, described in Quah (loc.cit).

In one aspect, the present invention relates to method of screening for a target of a DDIA which is resistant against said DDIA or has reduced susceptibility to said DDIA, said method comprises the steps of
(a) providing (e.g. in vitro/by recombinant techniques) (a library of) (a) gene(s)/(an) allele(s) of one or more (potential) target(s) of a given DDIA with one or more mutations (e.g. point mutations and/or (small) deletions);
(b) introducing said (a library of) mutated gene(s)/allele(s) of one or more (potential) target(s) of a given DDIA as provided according to step (a) into a (pool of) (immune) cell(s) (preferably a (pool of) naïve (immune) cell(s));
(c) contacting said (pool of) (immune) cell(s) with a/said given DDIA;
(d) selecting an (immune) cell which is resistant against said DDIA or has reduced susceptibility to said DDIA;
(e) recovering the cell which has been selected according to step (d); and
(f) recovering from said cell as recovered according to step (d) (optionally including sequencing) said mutated gene(s)/allele(s) of one or more (potential) target(s) of a given DDIA, thereby identifying said target of a DDIA which is resistant against said DDIA or has reduced susceptibility to said DDIA.

In the context of the above-described method of screening for a resistant/less susceptible target of a DDIA, a pool of cells with a mutagenized potential target may be generated according to, for example, Neggers (loc.cit.). What has been said with respect to the other above-described screening method also applies here, mutatis muntandis. Likewise, the skilled person can also rely on the references and respective means and methods mentioned in this respect and herein elsewhere.

The above-described method of screening may further comprise the step of
(g) testing/confirming whether said mutated gene(s)/allele(s) of one or more (potential) target(s) of a given DDIA is(are) capable of conferring resistance or reduced susceptibility to said (immune) cell (e.g. by contacting said (immune) cell with said DDIA and assaying the activity of the target encoded by said mutated gene(s)/allele(s)).

Any of the above-described methods of screening may further comprise the step of identifying the target(s) with the highest resistance against said DDIA or lowest susceptibility to said DDIA, respectively.

Further, in the context of any of the above-described methods of screening, said one or more mutations may be introduced into said (a) gene(s)/(an) allele(s) of one or more (potential) target(s) of a given DDIA by using CRISPR/Cas (e.g. including the use of sgRNAs) and/or by using (lenti)viruses (e.g. expressing sgRNAs) (see, for example, Neggers (loc.cit.), Clue (loc.cit.), Prommersberger (loc.cit.)).

Any of the methods of screening according to the invention may further comprise the step of evaluating said screened target(s) in an (animal) model, for example in an (animal) model for tumor/cancer. Suitable (animal) models are known in the art and are described in the appended examples. For example, a suitable cellular model of PDAC are KPC cells (for example as described in Hingorani S R, Wang L, Multani A S, Combs C, Deramaudt T B, Hruban R H, Rustgi A K, Chang S, Tuveson D A (2005) Trp53R172H and KrasG12D cooperate to promote chromosomal instability and widely metastatic pancreatic ductal adenocarcinoma in mice. Cancer cell 7: 469-483). An example of a cellular model for CRC are cultured (human) colon cancer cells and (human) colon cancer organoids.

Suitable animal models to be employed in the context of the invention are known in the art (e.g. Roeschert loc.cit.) and are described herein elsewhere and in the appended examples (e.g. Examples 1 (e.g. “Model Systems”), and Example 2).

In the context of the methods of screening according to the invention,
(i) said (immune) cell may be a T-cell (preferred) or a natural killer (NK) cell, or a progenitor cell thereof, for example a cell as described herein elsewhere;
(ii) said target may be a target as defined herein elsewhere and/or
(iii) said DDIA may be a DDIA as defined herein elsewhere.

In one aspect, the present invention relates to a method of screening for an agent (DDIA) that is capable of inhibiting a target in a cell of a cancer/tumor and thereby inducing DNA damage and/or preventing resolution of DNA damage in said cell of a cancer/tumor, and that is incapable of inhibiting a mutant of said target which is resistant against said agent (DDIA) or has reduced susceptibility to said agent in an (immune) cell or progenitor cell thereof and thereby not inducing DNA damage and/or preventing resolution of DNA damage in said (immune) cell or progenitor cell thereof. Said method may comprise the steps of:
(a) contacting a cell of a cancer/tumor with an immune cell or progenitor cell thereof; and
(b) contacting said cell of a cancer/tumor and immune cell or progenitor cell thereof with the/an agent to be screened; and/or
(c) determining the growth/progression/proliferation of said cell of a cancer/tumor and/or the growth/progression/proliferation of said (immune) cell or progenitor cell thereof and/or assaying the activity of said target and/or mutant of said target.

A reduced growth/progression/proliferation of said cell of a cancer/tumor and/or an increased, less reduced or unimpaired growth/progression/proliferation of said (immune) cell or progenitor cell thereof and/or a less reduced or unimpaired activity of said mutant of said target, as compared to a control, is indicative of said agent being capable of inhibiting a target in a cell of a cancer/tumor and thereby inducing DNA damage and/or preventing resolution of DNA damage in said cell of a cancer/tumor and/or that is incapable of inhibiting a mutant of said target which is resistant against said agent or has reduced susceptibility to said agent in an (immune) cell or progenitor cell thereof and thereby not inducing DNA damage and/or preventing resolution of DNA damage in said (immune) cell or progenitor cell thereof.

“Reduced” growth/progression/proliferation etc. in this context means, for example, ≤5%, ≤10%, ≤20%, ≤30%, ≤40%, ≤50%, ≤75% or 90% of the growth/progression/proliferation etc. of a control (e.g. cancer/tumor cell and/or immune/progenitor cell) which has not been contacted with the DDIA and/or the (immune/progenitor) cell.

“Increased”, “less reduced”, “unimpaired” growth/progression/proliferation or activity means, for example, ≤5%, ≤10%, ≤20%, ≤30%, ≤40%, ≤50%, ≤75% or 90% as compared to a control (e.g. cancer/tumor cell and/or immune/progenitor cell which has not been contacted with the DDIA and/or the (immune/progenitor) cell.

In the context of the above method of screening for an agent, screening of cells/cell lines for drug (DDIA) sensitivity may be performed, as, for example, described in Barrentina (loc.cit.). What has been said with respect of the other above-described screening methods also applies here, mutatis mutandis. Likewise, the skilled person can also rely on the references and respective means and methods mentioned in this respect.

The methods of screening of the invention, in particular the methods of screening of an agent (DDIA), may further comprise the step of introducing (transplanting) the/a cell of a cancer/tumor and/or the/a (immune) cell or progenitor cell thereof into an (immune-compromised) (animal) model (e.g. C57BL/6 mice (preferred) or nude mice), or further comprising the use of an (immune-compromised) (animal) model (e.g. C57BL/6 mice (preferred) or nude mice) which comprises/carries said cell of a cancer/tumor and/or said immune cell or progenitor cell thereof. In this context, an improved score (e.g. survival of said (animal) model (as compared to a control) may be indicative of said agent being capable of inhibiting a target in a cell of a cancer/tumor and thereby inducing DNA damage and/or preventing resolution of DNA damage in said cell of a cancer/tumor and/or that is incapable of inhibiting a mutant of said target which is resistant against said agent or has reduced susceptibility to said agent in an immune cell or progenitor cell thereof and thereby not inducing DNA damage and/or preventing resolution of DNA damage in said immune cell or progenitor cell thereof.

“Improved” score in this context means, for example, ≤5%, ≤10%, ≤20%, ≤30%, ≤40%, ≤50%, 75% or 90% higher as in a control (e.g. cancer/tumor cell and/or immune/progenitor cell which has not been contacted with the DDIA and/or the (immune/progenitor) cell. The skilled person is able to choose suitable scores. For example, a suitable score may be survival of the animal or cellular model (e.g. in days (from the application of the agent on)), growth/dimension/volume of a tumor, amount of tumor cells, and the like.

In any of the methods of screening of the invention, in particular of a method of screening for an agent, the (model) cell/organoid of a cancer/tumor may express, or may be genetically engineered to express, a particular (human) antigen (e.g. B7H3 (preferred) or ROR1; or another well known human antigen). This renders the method of screening independent of any specific (immune) cell binding to a particular cancer/tumor antigen, like any specific CAR T-cell; and results in relyable and comparable results. In any of the methods of screening of the invention, in particular of a method of screening for an agent, the (immune) cell or progenitor cell thereof may express a receptor that specifically binds to the particular (human) antigen (e.g. B7H3 or ROR1). Recombinant T-cell receptors, artificial T-cell receptors and especially CARs are particularly preferred in this respect.

In the context of a screening method of the invention, in particular of a method of screening for an agent,
(i) the cancer/tumor may be a cancer/tumor as defined herein elsewhere;
(ii) the (immune) cell or progenitor cell thereof may be an (immune) cell or progenitor cell thereof as defined herein elsewhere;
(iii) the receptor may be a receptor as defined wherein elsewhere, preferably a CAR;
(iv) the (animal) model may be a mouse (preferred), a rat, a rabbit, a monkey, or another suitable laboratory animal model;
(v) the agent may be a DDIA as defined herein elsewhere;
(vi) the target may be a target as defined herein elsewhere or a target which is encoded by a mutated gene(s)/allele(s) of a target as defined herein elsewhere; and/or
(vii) the cell of a cancer/tumor may be a model cell of a cancer/tumor as defined herein elsewhere (e.g. a KPC cell (or arganoid) or a model cell (or arganoid) of CRC).

In principle, any suitable model (cellular model, or organoid model, animal model, etc.) may be used in accordance with the invention, in particular in the context of the methods of screening described herein. The person skilled in the art is able to chose (a) suitable model(s) when performing such a screening method. In particular, respective models are models of tumor/cancers, for example as described herein elsewhere, and models of (immune) cells (or progenitors thereof), for example as described herein elsewhere.

Particular examples of cellular/organoid models are:
cellular models of PDAC, e.g. KPC cells (e.g. as described in Hingorani loc.cit.);
CRC cells or organoids.

Animal models to be used may be syngeneic animals (e.g. mice), immune-compromised animals (e.g. mice) or immunocompetent animals (e.g. mice).

Examples of particular animal models which may be employed in accordance with the invention are:
TH-MYCN mice (Roeschert loc.cit.);
B6: C57BL/6 mice;
Rag1: Rag 1^{-/-} mice;
NRG mice;
syngeneic mice based on a metastatic murine cell line, carrying KRAS, p53, APC, and TGF beta mutations (Tauriello, Nature 554, 2018, 538-43).

A particular model system to be used in accordance with the invention are KPC cells (e.g. for screening MYC-dependent tumor/cancers like PDAC or neuroblastoma). Another particular model, in particular for measuring growth of metastases (e.g. during liver regeneration) are CRC cells/organoids.

Any of the methods of screening (or any other method) as described herein may be performed in vivo (at least partially, e.g. at least one or more of the respective steps) or in vitro (at least partially, e.g. at least one of the respective steps). In particular, when relying on cellular models or organoid models, the methods (e.g. one or more of the respective steps) may be performed in vitro. In particular, when relying on animal models, the screening methods (e.g. one or more of the respective steps) may be performed in vivo.

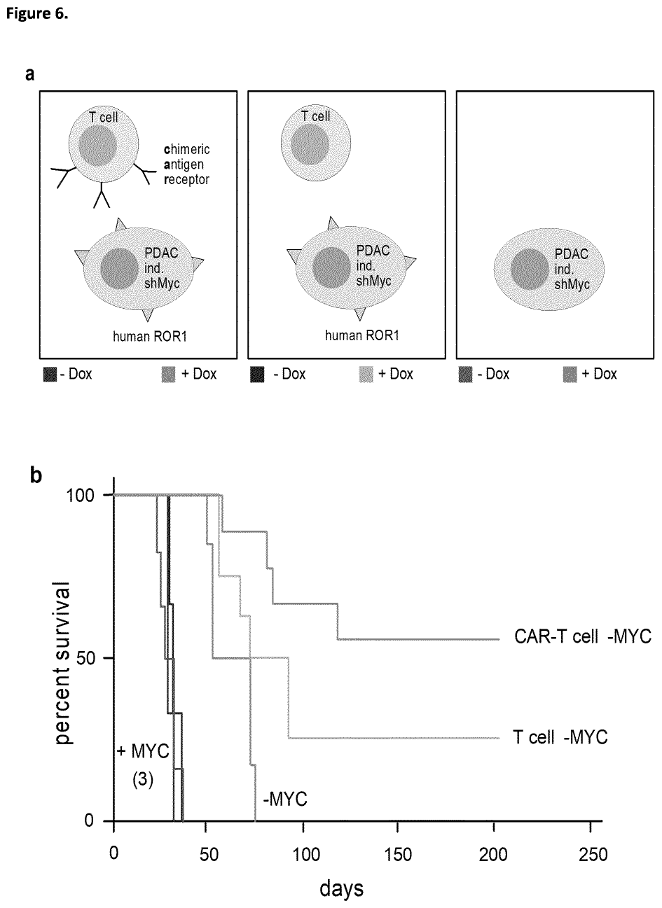

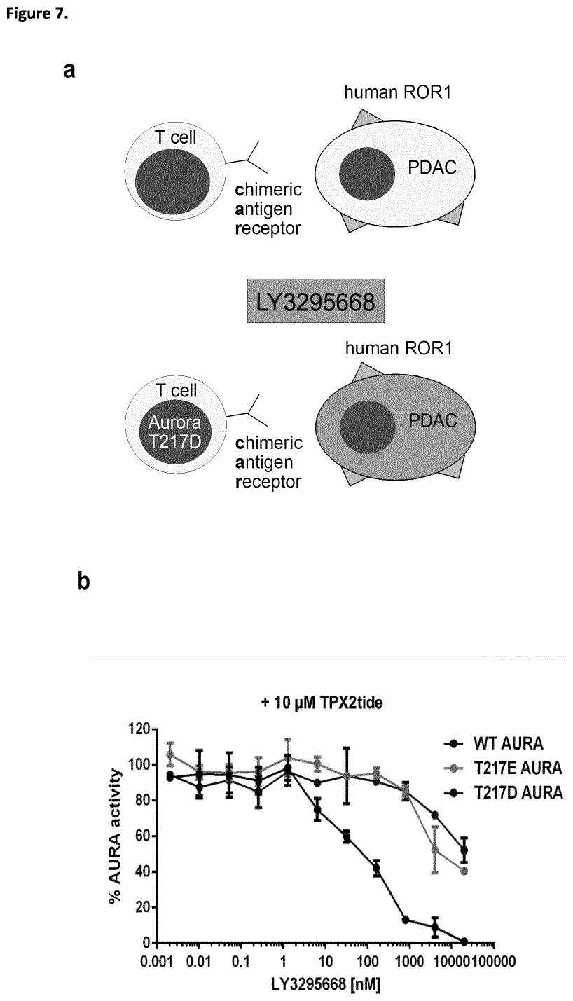

A general scheme, according to which the treatment methods (e.g. hybrid tumor therapy) and the screening methods according to the invention may be employed, is depicted in the appended FIG. 7a, and is also described in Example 7. The T cell depicted therein may also be any other (immune) cell or progenitor as described herein, the DDIA resistant/less susceptible Aurora-A kinase may be any other DDIA resistant/less susceptible target as described herein, the (model) tumor/cancer cell as depicted therein may be any other (model) tumor/cancer cell as described herein, the CAR as depicted herein may be any other receptor as described herein and/or the (human) antigen as depicted therein may be any other (human) antigen as described herein.

The present invention also relates to the following items:
1. An immune cell, or a progenitor cell thereof, which is resistant against a DNA damage-inducing agent (DDIA) or which exhibits reduced susceptibility to a DDIA.
2. The immune cell according to item 1, which is a T-cell (preferred) or a natural killer (NK) cell, or the progenitor cell according to item 1 which is a hemocytoblast ((omni- or multipotent) hematopoietic stem cell), a common lymphoid progenitor, a common myeloid progenitor, a lymphoblast or a myeloblast.
3. The immune cell according to item 1 or 2, which expresses a recombinant T-cell receptor and/or an artificial T-cell receptor.
4. The immune cell according to any one of items 1 to 3, which expresses a chimeric antigen receptor (CAR).
5. The immune cell according to item 3 or 4, wherein said receptor specifically binds to a tumor antigen.
6. The immune cell according to any one of items 3 to 5, wherein said receptor specifically binds to a tumor-specific antigen (TSA) or to a tumor-associated antigen (TAA).
7. The immune cell according to any one of items 1 to 6, wherein said immune cell is a CAR T-cell.
8. The immune cell or progenitor cell thereof according to any one of items 1 to 7, which is made resistant against said DDIA by (genetical) engineering or has reduced susceptibility to said DDIA due to (genetical) engineering.
9. The immune cell or progenitor cell thereof according to any one of items 1 to 8, which comprises at least one target of said DDIA which is resistant against said DDIA or has reduced susceptibility to said DDIA.
10. The immune cell or progenitor cell thereof according to item 9, wherein said target carries a mutation, or two or more mutations, which renders/render said target as being resistant against said DDIA or as having reduced susceptibility to said DDIA.
11. The immune cell or progenitor cell thereof according to item 9 or 10, which comprises at least one allele of said target, wherein said allele carries a mutation, or two or more mutations, which renders/render said target resistant against said DDIA or as having reduced susceptibility to said DDIA.
12. The immune cell or progenitor cell thereof according to any one of items 1 to 11, wherein said resistance or reduced susceptibility against said DDIA is a conditional resistance and reduced susceptibility, respectively (e.g. a resistance and reduced susceptibility, respectively, which is conditional to a FKB analogue, to auxin or an auxin derivative or to a steroid hormone).
13. The immune cell or progenitor cell thereof according to item 12, wherein said resistance or reduced susceptibility is conditional to doxycycline.
14. The immune cell or progenitor cell thereof according to item 13, wherein said target of said DDIA is conditionally expressed in the presence of doxycycline.
15. A pharmaceutical composition comprising the immune cell and/or a progenitor cell thereof according to any one of items 1 to 14.
16. A pharmaceutical composition, a kit or a combination (set of two/three components) comprising (e.g. in two/three different vials)
   (i) an immune cell and/or a progenitor cell thereof according to any one of items 1 to 14; and
   (ii) said DDIA.
17. A pharmaceutical composition according to item 15, the pharmaceutical composition, kit or combination according to item 16 or (a combination of)
   (i) an immune cell and/or a progenitor cell thereof according to any one of items 1 to 14; and
   (ii) said DDIA
   for use in treating a cancer and/or a tumor.
18. The pharmaceutical composition, kit, combination or (combination of) immune cell and/or progenitor cell thereof and DDIA for use according to item 17, wherein said tumor is a malignant and/or metastasizing tumor.
19. The pharmaceutical composition, kit, combination or (combination of) immune cell and/or progenitor cell thereof and DDIA for use according to item 17 or 18, wherein said treating comprises the treating of metastases and/or the prevention (of the growth) of metastases.
20. The pharmaceutical composition, kit, combination or (combination of) immune cell and/or progenitor cell thereof and DDIA for use according to any one of items 17 to 19, wherein said tumor is a solid tumor.
21. The pharmaceutical composition, kit, combination or (combination of) immune cell and/or progenitor cell thereof and DDIA for use according to any one of items 17 to 20, wherein said cancer and/or tumor is a Myc-driven cancer and/or tumor (e.g. a c-Myc-, L-Myc- and/or N-Myc-driven cancer and/or tumor).
22. The pharmaceutical composition, kit, combination or (combination of) immune cell and/or progenitor cell thereof and DDIA for use according to any one of items 17 to 21, wherein said cancer and/or tumor is
   (i) a pancreas cancer/carcinoma and/or tumor, in particular pancreatic ductal adenocarcinoma (PDAC);
   (ii) a colon cancer/carcinoma and/or tumor, in particular metastatic colorectal carcinoma (CRC); or
   (iii) (pediatric) neuroblastoma.
23. The pharmaceutical composition, kit, combination or (combination of) immune cell and/or progenitor cell thereof and DDIA for use according to any one of items 17 to 22, in particular according to item 22 (ii), wherein said treating comprises the treating of metastases and/or the prevention (of the growth) of metastases in the liver.
24. The immune cell or progenitor cell thereof according to any one of items 1 to 14, the pharmaceutical composition according to item 15, the pharmaceutical composition, kit or combination according to item 16 or the pharmaceutical composition, kit, combination or (combination of) immune cell and/or progenitor cell thereof and DDIA for use according to any one of items 17 to 23, wherein said DDIA is a transcription-replication conflict-inducing agent (TRCIA) (this is envisaged to include agents which prevent/target resolution of TRCs).
25. The immune cell or progenitor cell thereof according to any one of items 1 to 14 and 24, the pharmaceutical composition according to item 15 or 24, the pharmaceutical composition, kit or combination according to item 16 or 24 or the pharmaceutical composition, kit, combination or (combination of) immune cell and/or progenitor cell thereof and DDIA for use according to any one of items 17 to 24 (in particular according to any one of items 21 to 23), wherein said DDIA targets Myc and/or results in a reduction/depletion of (the expression of) Myc.
26. The immune cell or progenitor cell thereof according to any one of items 1 to 14, 24 and 25, the pharmaceutical composition according to any one of items 15, 24 and 25, the pharmaceutical composition, kit or combination according to any one of items 16, 24 and 25 or the pharmaceutical composition, kit, combination or (combination of) immune cell and/or progenitor cell thereof and DDIA for use according to any one of items 17 to 25, wherein the target of said DDIA is a target selected from the group consisting of
   (i) Aurora A kinase;
   (ii) Ataxia telangiectasia and Rad3-related (ATR) kinase;
   (iii) PAFc complexes;
   (iv) PAF1 (e.g. CDC73, LEO1, CTR9);
   (v) CDK9;
   (vi) CDK12; CDK13
   (vii) cyclinK/CDK12 complexes;
   (viii) splicing factors (e.g. SF3B1, RBM39) and/or transcription termination factors; e.g. SPT5, EXOsome
   (ix) SNRNP70;
   (x) CPSF1; CPSF2
   (xi) CPSF3;
   (xii) PNUTs/PP11 phosphatase complex;
   (xiii) NUAK1/ARK5;
   (xiv) RNA polymerase 1 (POL1);
   (xv) ATM kinase;
   (xvi) Na⁺/K⁺-ATPase, in particular human Na⁺/K⁺-ATPase;
   (xvii) USP28;
   (xviii) Topoisomerase I;
   (xix) Topoisomerase II; and
   (xx) Poly(ADP-ribose)-Polymerase.
27. The immune cell or progenitor cell thereof according to any one of items 1 to 14 and 24 to 26, the pharmaceutical composition according to any one of items 15 and 24 to 26, the pharmaceutical composition, kit or combination according to any one of items 16 and 24 to 26 or the pharmaceutical composition, kit, combination or (combination of) immune cell and/or progenitor cell thereof and DDIA for use according to any one of items 17 to 26, wherein said DDIA is selected from the group consisting of
   (i) an Aurora A kinase inhibitor (e.g. MLN8054, MLN8237 (Alisertib; Millennium), LY3295668);
   (ii) an ATR kinase inhibitor (e.g. AZD6738 (Astra-Zeneca), BAY 1895344 (Bayer));
   (iii) a PAFc complex inhibitor or an inhibitor of any subunit of the PAFc complex;
   (iv) a CDK9 inhibitor (e.g. AZD4573, NVP-2, CYC065 (fadracicikb), THAL-SNS-03);
   (v) a CDK12 inhibitor (e.g. SR4835, THZ-531);
   (vi) a cyclinK/CDK12 complexes inhibitor (e.g. CR-8);
   (vii) a splicing and/or termination complexes inhibitor (e.g. insidulam, SPI-21 (Bahat, Mol Cell 76, 2019, 617-31 e614), Pladienolide B, H3B-8800)
   (viii) a SNRNP70 inhibitor (e.g. . . . );
   (ix) a CPSF1 inhibitor (e.g. . . . );
   (x) a CPSF3 inhibitor (e.g. JTE-607);
   (xi) a PNUTs/PP11 phosphatase complex inhibitor (e.g. calyculin A);
   (xii) a NUAK1/ARK5 inhibitor (e.g. BAY-880 (Bayer), ON-123300, XMD-1571, HTH-01-015);
   (xiii) a POL1 inhibitor (e.g. CX-5461);
   (xiv) ATM kinase inhibitor (e.g. KU-60019, KU-559403, AZD1390);
   (xv) a (human) Na⁺/K⁺-ATPase inhibitor (e.g. coumarin, ouabain, digitoxin, cymarin, digoxin, acetyldigitoxin, deslanoside.);
   (xvi) a USP28 inhibitor (e.g. FT206, AZ1);
   (xvii) Topoisomerase I inhibitor (e.g. Irinotecan, topotecan, campthotecin);
   (xviii) Topoisomerase II inhibitor (e.g. etoposide, doxorubicin, daunorubicin); and
   (xix) Poly(ADP-ribose)-Polymerase inhibitor (e.g. olaparib, veliparib).
28. The pharmaceutical composition according to any one of items 15 and 24 to 27, the pharmaceutical composition, kit or combination according to any one of items 16 and 24 to 27 or the pharmaceutical composition, kit, combination or (combination of) immune cell and/or progenitor cell thereof and DDIA for use according to any one of items 17 to 27, wherein at least two different DDIAs are to be administered.
29. The pharmaceutical composition according to any item 28, the pharmaceutical composition, kit or combination according to item 28 or the pharmaceutical composition, kit, combination or (combination of) immune cell and/or progenitor cell thereof and DDIA for use according to item 28, wherein one of said two different DDIAs is (a low dose of) an ATR kinase inhibitor (preferred) or (a low dose of) an ATM kinase inhibitor.
30. The immune cell or progenitor cell thereof according to any one of items 9 to 14 and 24 to 27, the pharmaceutical composition according to any one of items 15 and 24 to 29, the pharmaceutical composition, kit or combination according to any one of items 16 and 24 to 29 or the pharmaceutical composition, kit, combination or (combination of) immune cell and/or progenitor cell thereof and DDIA for use according to any one of items 17 to 29, wherein said at least one target of said DDIA which is resistant against said DDIA or has reduced susceptibility to said DDIA and said DDIA, respectively, are selected from the group consisting of
   (i) Aurora A kinase T217E or T217D mutant (or another DDIA-resistant Aurora A kinase mutant) and MLN8054, MLN8237 or LY3295668, respectively (e.g. for use in the treatment of (pediatric) neuroblastoma);
   (ii) CDK12 C1039S mutant and THZ-531, respectively (e.g. for use in the treatment of CDK12-dependent tumors, like triple-negative breast cancer/tumor);
   (iii) RBM39 G268V mutant and indisulam, respectively (e.g. for use in the treatment of MYC or MYCN-driven tumors, like colon, pancreatic and small cell lung cancers/tumors);
   (iv) murine Na⁺/K⁺-ATPase (or another CG-resistant Na⁺/K⁺-ATPase; see herein elsewhere) and coumarin, oubain, digitoxin, cymarin, digoxin, acetyldigitoxin, deslanoside, or another (human) Na⁺/K⁺-ATPase inhibitor (e.g. another CG), respectively (e.g. for use in the treatment of MYC-dependent cancers/tumors, like colon and pancreatic cancers/tumors);
   (v) topoisomerase I with (a) mutation(s) that confer(s) resistance to (a) topoisomerase I inhibitor(s) and a topoisomerase I inhibitor, respectively, e.g. a topoisomerase I F361S, G363C and/or R364H mutant and campthotecin, respectively; or a topoisomerase I S365G, R621H and/or E710G mutant and irinotecan, respectively (e.g. for use in the treatment of . . . cancers/tumors);
   (vi) topoisomerase II with (a) mutation(s) that confer(s) resistance to (a) topoisornerase inhibitor(s) and a topoisomerase II inhibitor, respectively, e.g. a topoisomerase II P501, G776 and/or K505 mutant and etoposide, doxorubicin or mitoxantron, respectively; and
   (vii) a deletion of the cellular PARP gene and a PARP inhibitor, respectively (e.g. olaparib or veliparib) (e.g. for use in the treatment of pediatric tumors).
31. The pharmaceutical composition according to any one of items 15, 16 and 24 to 30 for use in controlling an immune cell therapy, wherein said immune cell therapy comprises the use of an immune cell or progenitor cell thereof according to any one of items 12 to 14, 24 to 27 and 30.
32. A method of screening for a target of a DDIA which is resistant against said DDIA or has reduced susceptibility to said DDIA, said method comprises the steps of:
   (a) generating a pool/library of (immune) cells with one or more mutations (e.g. point mutations and/or (small) deletions) in (a) gene(s)/(an) allele(s) of one or more (potential) target(s) of a given DDIA;
   (b) contacting said pool/library of (immune) cells with a/said given DDIA;
   (c) selecting cells of said pool/library of (immune) cells which are resistant against said DDIA or have reduced susceptibility to said DDIA (as compared to a control);
   (d) recovering the cells which have been selected according to step (c); and
   (e) recovering from said cells as recovered according to step (d) (optionally including sequencing) said mutated gene(s)/allele(s) of one or more (potential) target(s) of a given DDIA, thereby identifying said target of a DDIA (or CG) which is resistant against said DDIA or has reduced susceptibility to said DDIA.
33. The method of screening according to item 32, further comprising the steps of
   (f) reintroducing said mutated gene(s)/allele(s) of one or more (potential) target(s) of a given DDIA as recovered according to step (e) into an (immune) cell (preferably a naïve (immune) cell); and/or
   (g) testing/confirming whether said mutated gene(s)/allele(s) of one or more (potential) target(s) of a given DDIA is capable of conferring resistance or reduced susceptibility to said (immune) cell (e.g. by contacting said (immune) cell with said DDIA and assaying the activity of the target encoded by said mutated gene(s)/allele(s)).
34. A method of screening for a target of a DDIA which is resistant against said DDIA or has reduced susceptibility to said DDIA, said method comprises the steps of
   (a) providing (e.g. in vitro/by recombinant techniques) (a library of) (a) gene(s)/(an) allele(s) of one or more (potential) target(s) of a given DDIA with one or more mutations (e.g. point mutations and/or (small) deletions);
   (b) introducing said (a library of) mutated gene(s)/allele(s) of one or more (potential) target(s) of a given DDIA as provided according to step (a) into a (pool of) (immune) cell(s) (preferably a (pool of) naïve (immune) cell(s));
   (c) contacting said (pool of) (immune) cell(s) with a/said given DDIA;
   (d) selecting an (immune) cell which is resistant against said DDIA or has reduced susceptibility to said DDIA (as compared to a control);
   (e) recovering the cell which has been selected according to step (d); and
   (f) recovering from said cell as recovered according to step (d) (optionally including sequencing) said mutated gene(s)/allele(s) of one or more (potential) target(s) of a given DDIA, thereby identifying said target of a DDIA which is resistant against said DDIA or has reduced susceptibility to said DDIA.
35. The method of screening according to item 34, further comprising the step of
   (g) testing/confirming whether said mutated gene(s)/allele(s) of one or more (potential) target(s) of a given DDIA is(are) capable of conferring resistance or reduced susceptibility to said (immune) cell (e.g. by contacting said (immune) cell with said DDIA and assaying the activity of the target encoded by said mutated gene(s)/allele(s)).
36. The method of screening according to any one of items 32 to 35, further comprising the step of identifying the target(s) with the highest resistance against said DDIA or lowest susceptibility to said DDIA.
37. The method of screening according to any one of item 32 to 36, wherein said one or more mutations are introduced into said (a) gene(s)/(an) allele(s) of one or more (potential) target(s) of a given DDIA by using CRISPR/Cas (e.g. including the use of sgRNAs) and/or lentiviruses (e.g. expressing sgRNAs).
38. The method of screening according to any one of item 32 to 37, further comprising the step of evaluating said screened target(s) in an (animal) model.
39. The method of screening according to any one of items 32 to 38, wherein
   (i) said (immune) cell is a T-cell (preferred) or a natural killer (NK) cell, or a progenitor cell as defined in item 1 or 2;
   (ii) said target is a target as defined in any one of items 10 to 12, 26 and 30; and/or
   (iii) said DDIA is a DDIA as defined in any one of items 24, 25 and 27.
40. A method of screening for an agent (DDIA) that is capable of inhibiting a target in a cell of a cancer/tumor and thereby inducing DNA damage and/or preventing resolution of DNA damage in said cell of a cancer/tumor, and that is incapable of inhibiting a mutant of said target which is resistant against said agent or has reduced susceptibility to said agent in an (immune) cell or progenitor cell thereof and thereby not inducing DNA damage and/or preventing resolution of DNA damage in said (immune) cell or progenitor cell thereof, said method comprises the steps of:
   (a) contacting a cell of a cancer/tumor with an (immune) cell or progenitor cell thereof; and
   (b) contacting said cell of a cancer/tumor and (immune) cell or progenitor cell thereof with the/an agent to be screened; and
   (c) determining the growth/progression/proliferation of said cell of a cancer/tumor and/or the growth/progression/proliferation of said (immune) cell or progenitor cell thereof and/or assaying the activity of said target and/or mutant of said target,
   wherein a reduced growth/progression/proliferation of said cell of a cancer/tumor and/or an increased, less reduced or unimpaired growth/progression/proliferation of said (immune) cell or progenitor cell thereof and/or a less reduced or unimpaired activity of said mutant of said target (as compared to a control) is indicative of said agent being capable of inhibiting a target in a cell of a cancer/tumor and thereby inducing DNA damage and/or preventing resolution of DNA damage in said cell of a cancer/tumor and/or that is incapable of inhibiting a mutant of said target which is resistant against said agent or has reduced susceptibility to said agent in an (immune) cell or progenitor cell thereof and thereby not inducing DNA damage and/or preventing resolution of DNA damage in said (immune) cell or progenitor cell thereof.
41. The method of screening according to item 40, further comprising the step of introducing (transplanting) said cell of a cancer/tumor and/or said (immune) cell or progenitor cell thereof into an (immune-compromised) (animal) model (e.g. C57BL/6 mice (preferred) or nude mice), or further comprising the use of an (immune-compromised) (animal) model (e.g. C57BL/6 mice (preferred) or nude mice) which comprises/carries said cell of a cancer/tumor and/or said (immune) cell or progenitor cell thereof, wherein an improved clinical score (e.g. survival) of said (animal) model (as compared to a control) is indicative of said agent being capable of inhibiting a target in a cell of a cancer/tumor and thereby inducing DNA damage and/or preventing resolution of DNA damage in said cell of a cancer/tumor and/or that is incapable of inhibiting a mutant of said target which is resistant against said agent or has reduced susceptibility to said agent in an (immune) cell or progenitor cell thereof and thereby not inducing DNA damage and/or preventing resolution of DNA damage in said (immune) cell or progenitor cell thereof.
42. The method of screening according to item 40 or 41, wherein said cell of a cancer/tumor expresses, or is (genetically) engineered to express, a particular (human) antigen (e.g. B7H3 (preferred) or ROR1).
43. The method of screening according to any one of items 40 to 42, wherein said (immune) cell or progenitor cell thereof expresses a receptor that specifically binds to the particular (human) antigen as defined in item 42.
44. The method of screening according to any one of items 40 to 43, wherein
   (i) said cancer/tumor is defined as in any one of items 18 to 23;
   (ii) said (immune) cell or progenitor cell thereof is an (immune) cell or progenitor cell thereof as defined in any one of items 1 to 14;
   (iii) said receptor is defined as in any one of items 3 to 6, preferably a CAR;
   (iv) said (animal) model is a mouse;
   (v) said agent is a DDIA as defined in any one of items 24, 25 and 27;
   (vi) said target is a target as defined in any one of items 9 to 12 and 30 or a target which is encoded by a mutated gene(s)/allele(s) of a target as defined in item 26;
   (vii) said agent is a DDIA as defined in any one of items 24, 25 and 27; and/or
   (viii) said cell of a cancer/tumor is a model cell of a cancer/tumor (e.g. a KPC cell or a model cell of CRC).

The present invention is further described by reference to the following non-limiting figures and examples.

The Figures show:

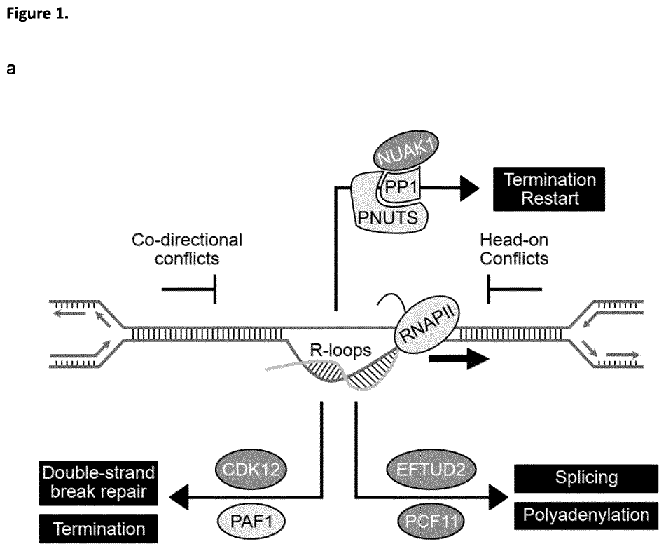

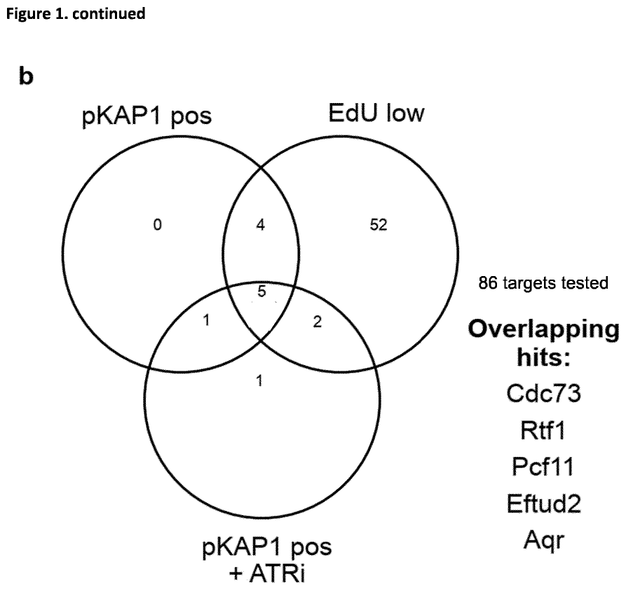

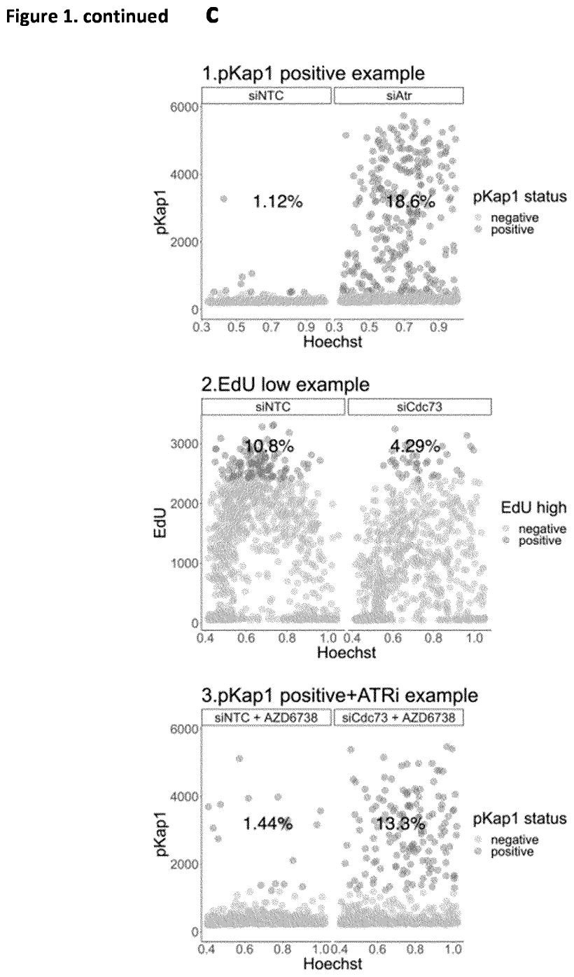

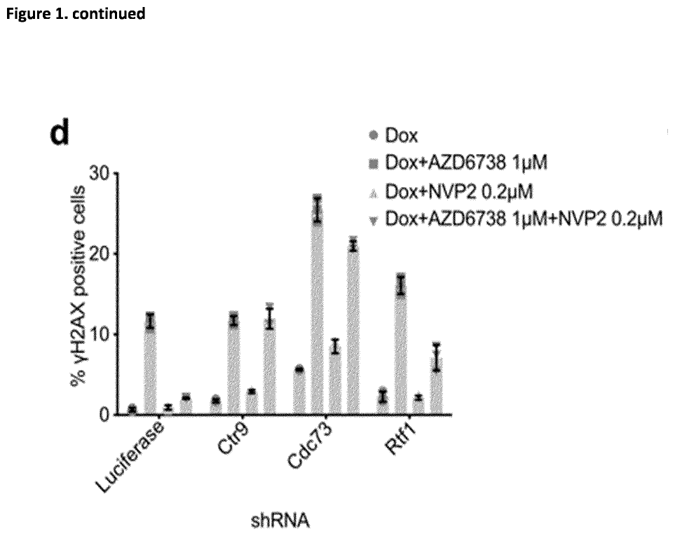

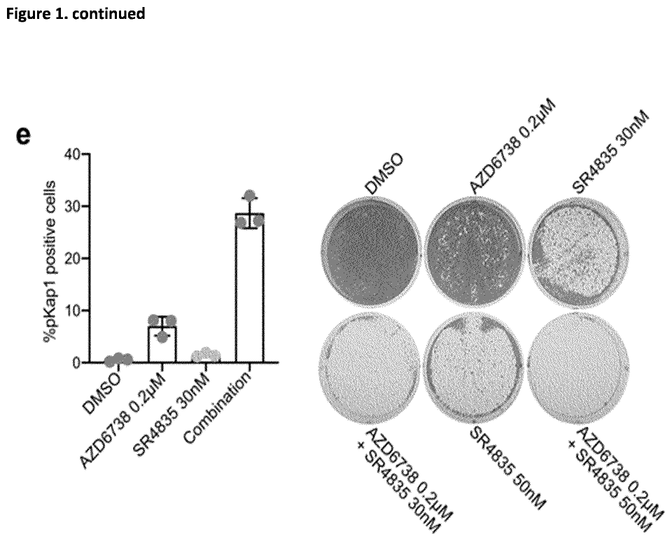

FIG. 1. Druggable pathways that resolve TRCs in pancreas carcinoma cells. a: Scheme of the pathways identified. b: Venn diagram showing the hits in three different microscopy-based assays out of 86 siRNAs screened in total. c: Assays used and examples for hits: top panel shows pKAP1-positive S-phase cells, indicating ATM activation in S-Phase; middle panel shows decrease in EdU incorporation, indicating decreased DNA synthesis; bottom shows strongly increased DNA damage in the presence of low concentration of ATR inhibitor (ATRi; AZD6738). d: Validation experiments: The panel shows the percentage of y-H2Ax-positive cells (cells with unrepaired DNA damage) upon incubation with low concentration of ATR inhibitor and shows that blockade of transcription elongation with a CDK9 inhibitor abolishes the damage, arguing it is due to elongation. In cells expressing shRNAs targeting PAF1 subunits, arresting RNA Polymerase before pause release does not protect from damage, demonstrating that PAF1 function is required before pause release to protect cells from replication stress. Similar assays (not shown) establish that CDK12 acts downstream of PAF1 in this pathway. e: DNA damage and colony assays in response to combined inhibition of CDK12 and ATR.

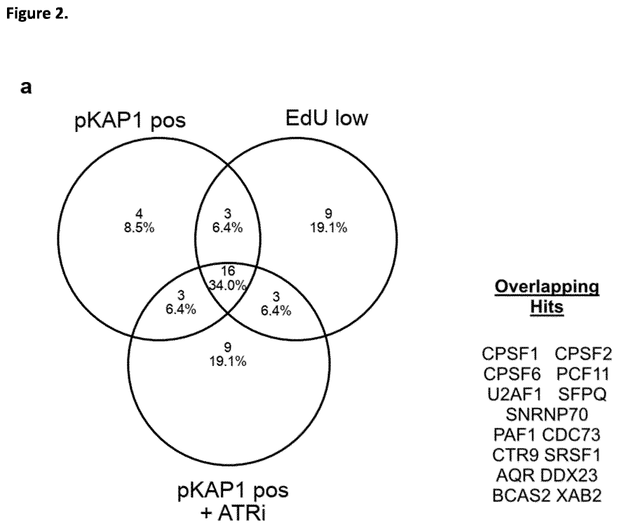

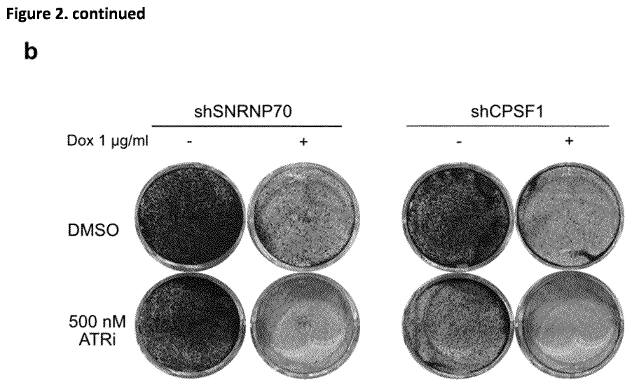

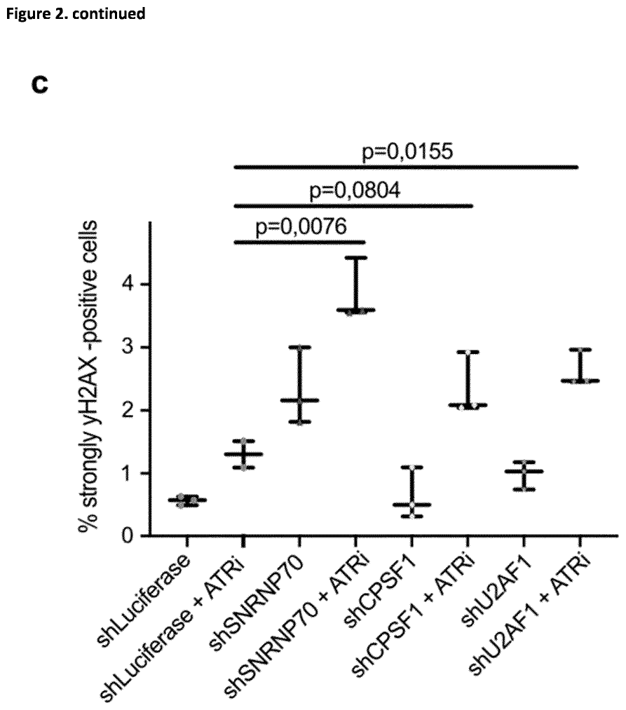

FIG. 2. Resolution of TRCs in colon carcinoma. a: Venn diagram illustrating hits using the same set of target genes and assays as in PDAC. b: Colony assays showing that depletion of a splicing protein, SNRNP70, and a polyadenylation factor, CPSF1, sensitizes colon tumor cells to low concentrations of ATR inhibitor. c: Quantitative gammaH2AX (gammaH2AX) immunofluorescence documenting induction of DNA damage by depletion of the indicated factors in conjunction with low concentrations of ATR inhibitor.

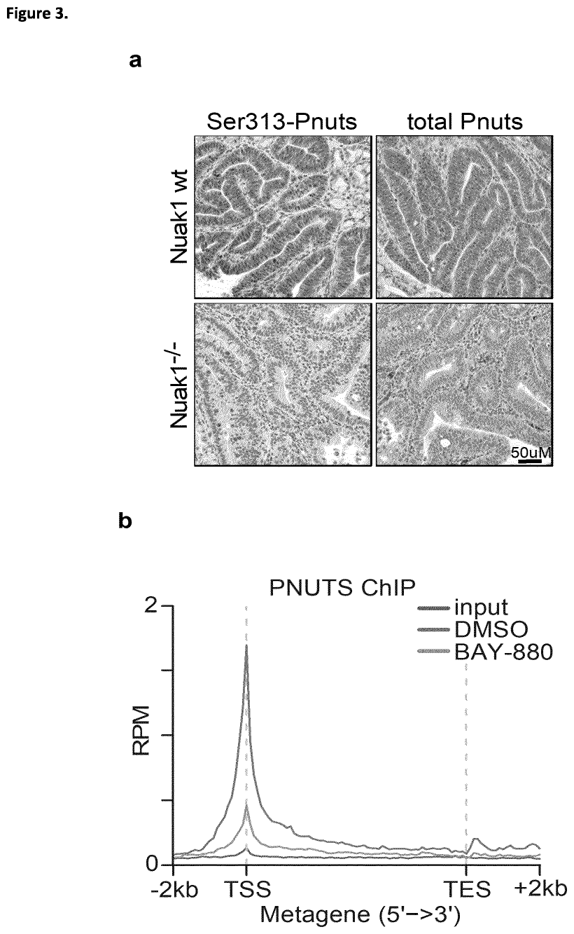

FIG. 3. Inhibition of NUAK1 causes TRCs. a: Histology of wildtype and NUAK1-deficient murine colon carcinoma showing absence of pS313 phosphorylation of PNUTS in NUAK1-deficient CRC. b: ChIP-sequencing data showing that chromatin association depends on NUAK1. Data show a metagene plot of all active genes (Cossa, Mol Cell 77, 2020, 1322-39). c: Proximity-ligation assays showing that NUAK1 inhibition increases the proximity between RNAPII and PCNA or pSer2 RNAPII and RAD9. d: Organoid assays of CRC showing that different NUAK1 inhibitors suppress growth of CRC organoids in conjunction with low dose ATR inhibitors; left: representative pictures; right: quantification of growth.

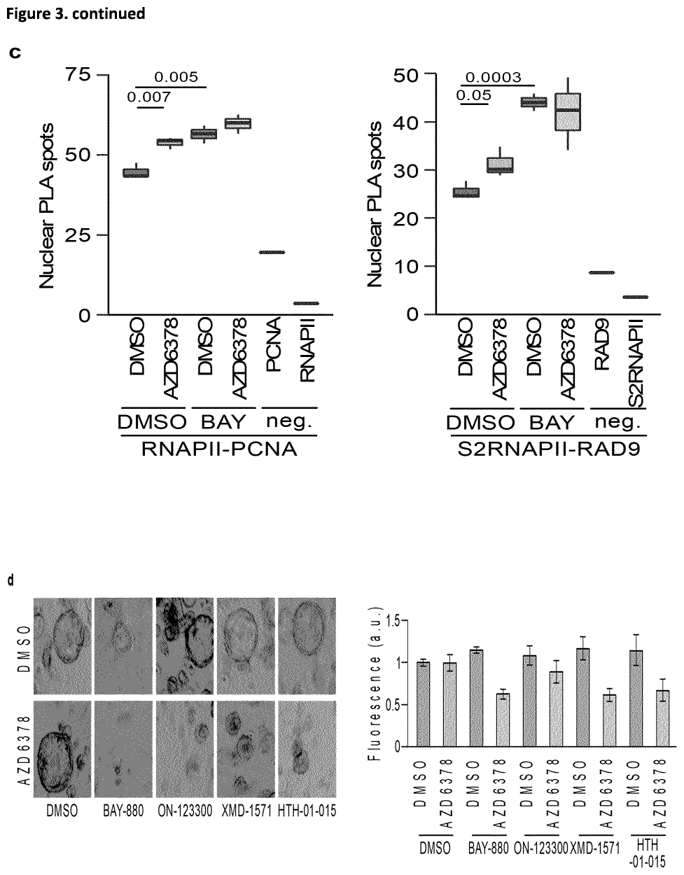

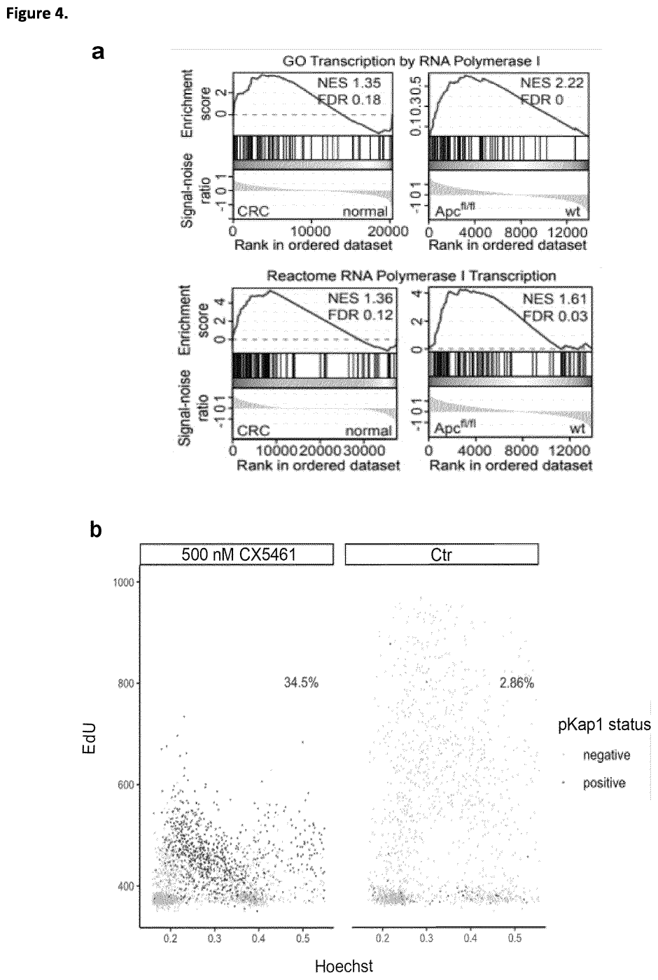

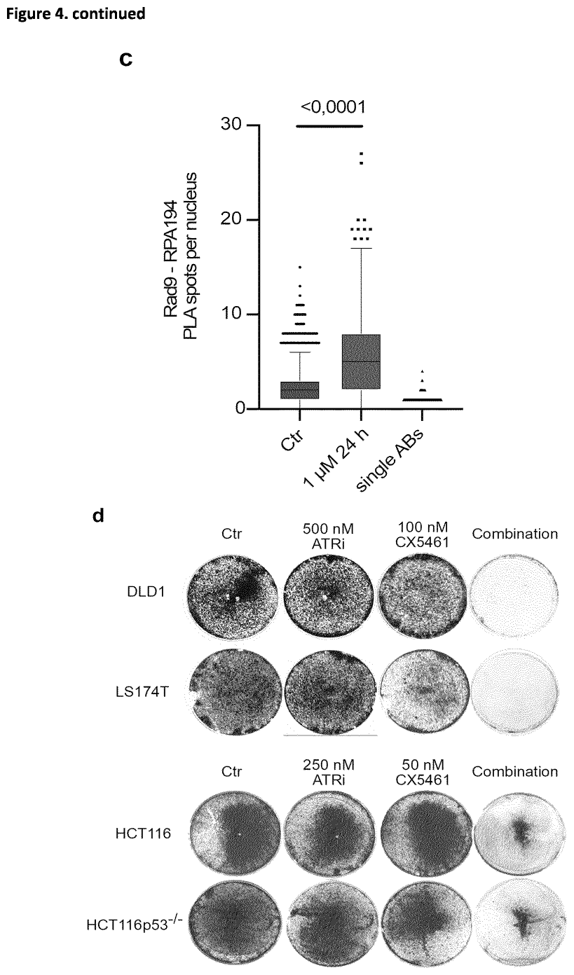

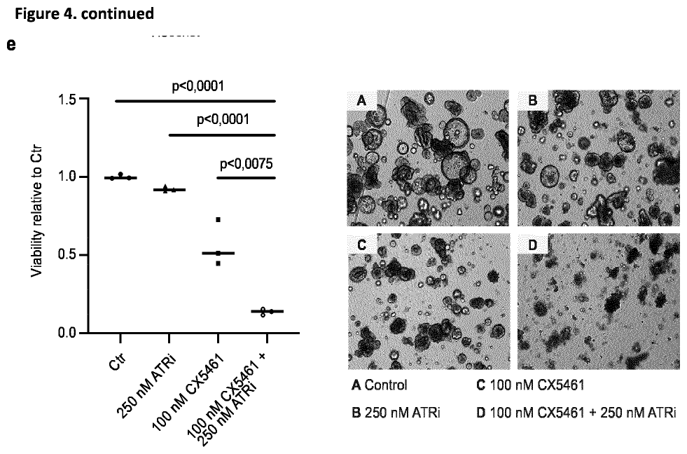

FIG. 4. Induction of TRCs by CX-5461. a: GSE analysis documenting APC-sensitive expression of the POL1 machinery (Schmidt, Nature cell biology 21, 2019, 1413-24). b: Two-dimensional EdU incorporation/Hoechst plots with phosphoKap1 positive cells stained in red showing induction of DNA damage by CX-5461 in S-phase. c: Proximity ligation assays for RPA194 and RAD9. d/e: Colony assays documenting suppression of growth of CRC cells in culture (d) and of CRC organoids (e) by combined ATR inhibition (AZD6738) and CX-5461.

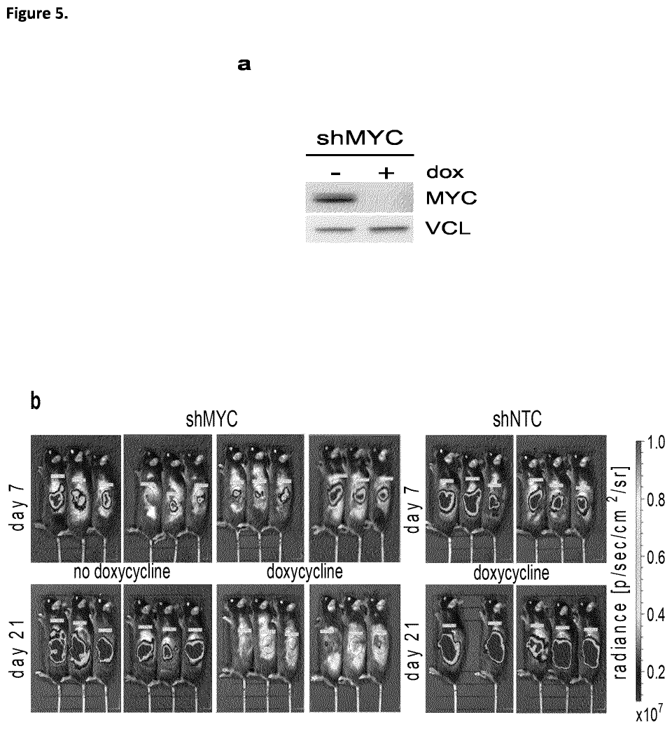

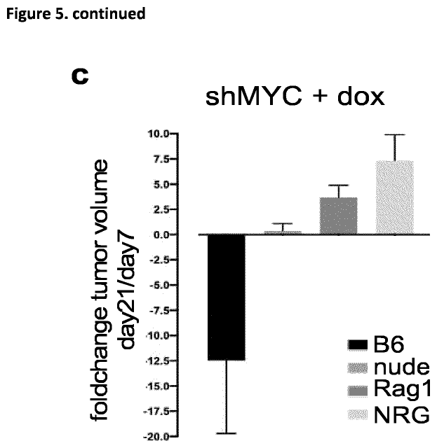

FIG. 5. T cell-mediated tumor regression of PDAC cells after MYC depletion. a: Immunoblots of KPC cells expressing shRNA targeting MYC. b: Tumor regression in immunocompetent mice as documented by luciferase imaging. c: Relative tumor growth during two weeks following MYC depletion upon transplantation of KPC cells into different host mice. B6: C57BL/6J mice (wt); Rag1: Rag1^{-/-} mice.

FIG. 6. Sensitization of PDAC growth to CAR T cells-mediated killing. a: Scheme of the experiments. KPC cells expressing a doxycyline-inducible are transduced to stably express ROR1 and mice are complemented with either naïve T-cells or a-ROR1 CAR T cells. b: Survival curves documenting no effect of CAR T cells in presence of MYC, but long-term survival upon MYC depletion.

FIG. 7. Hybrid T-cell/tumor cell therapies. a: Scheme of the experiments (see also text e.g. Example 7) b: Aurora-A kinase assays demonstrating that the T217D and T217E alleles confer resistance to LY3295668.

FIG. 8. Liver metastasis model and experimental surgery. a: Transplantation of CRC cells from organoids directly into the liver, leading to growth of a single “metastasis” in the liver. b: Multifocal growth of metastases after injection into spleen. c: Liver regeneration after resection. The upper panels show the resection, the lower panels document regeneration/hypertrophy after resection.

FIG. 9. Overview of cells which may be provided/used in accordance with the invention. The Figure is extracted from https://en.wikipedia.org/wiki/Haematopoiesis (Jun. 26, 2021).

FIG. 10. Mutation of AURKA rescues the effect of AURKA inhibition. a: Immunoblot of parental murine NHO2A cells compared to cells overexpressing (murine) AURKA*^{t}, (murine) AURKA^{T208D} or (murine) AURKA^{T208E}. b: Colony formation assay comparing (murine) AURKA*^{t}with (murine) AURKA^{T208D} expressing cells upon treatment with indicated concentrations of LY3295668 (AK01). c: BrdU/PI-FACS comparing (murine) AURKA*^{t} with (murine) AURKA^{T208D} expressing cells upon treatment with 1 μM AK01.

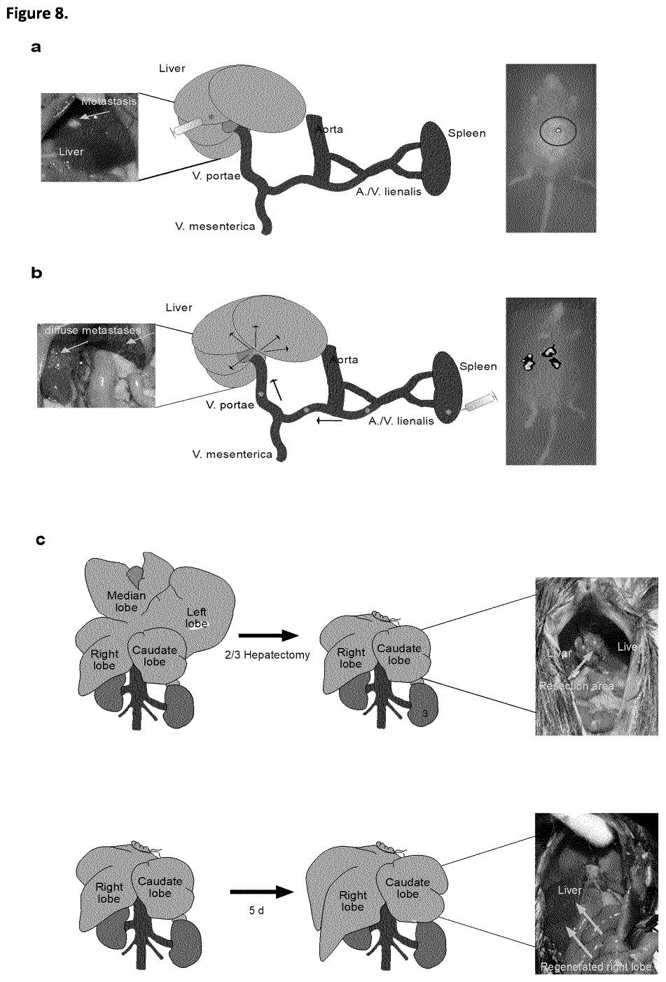

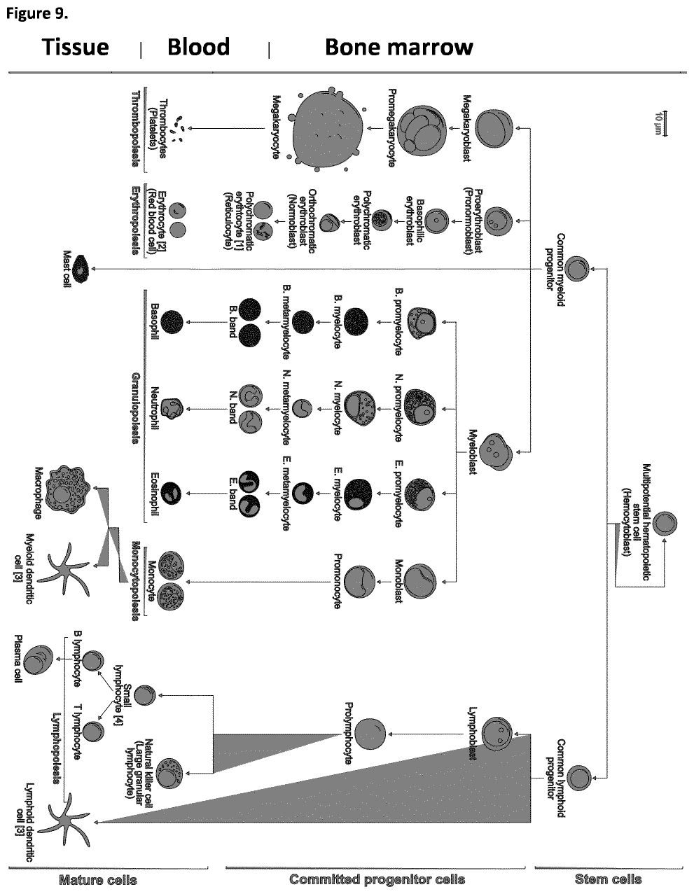

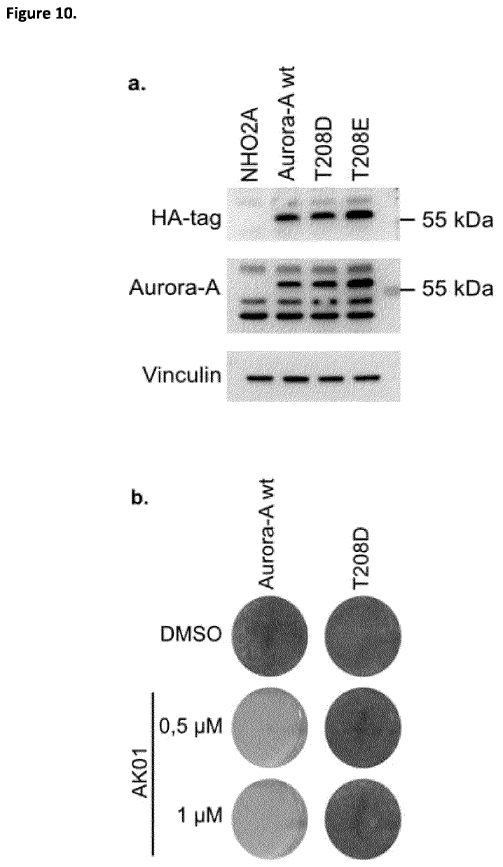

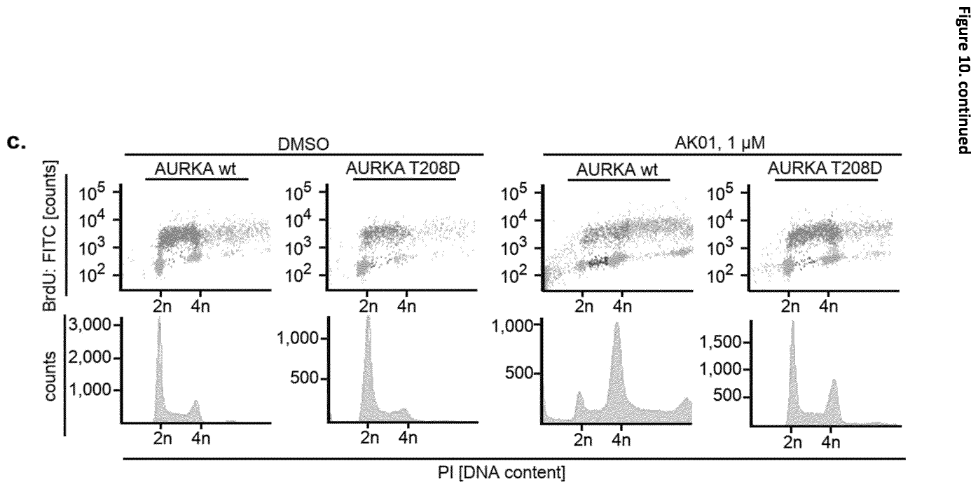

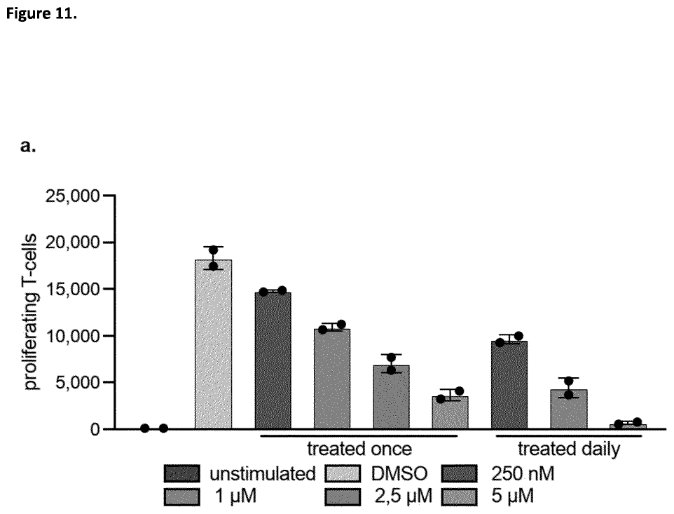

FIG. 11. Aurora-A inhibition impairs T-cell proliferation and activity. a: Quantification of proliferating T-cells upon 96 h of Aurora-A inhibitor treated only once at the beginning or daily (right). b: FACS measurement of activation markers CD69 (top) or CD25 (bottom) on the surface of CD8⁺ T cells upon treatment with indicated inhibitors.

FIG. 12. Efficacy of the hB7H3 CARs on eliminating tumor cells overexpressing hB7H3 in cocultivation assay. a: FACS staining showing T-cells (top)/CARs (bottom) (green; left squares) and tumor cells (orange; right squares) after co-cultivation for 72 h at the indicated effector to target ratio. b: Retroviral vector construct including the AURKA domain within the CAR construct.

FIG. 13. Role of Cardiac Glycosides (CGs). left: Tumor cells secrete immune suppressive lactate. middle: CGs inhibit lactate secretion & affect viability of T-cells. right: CGs inhibit lactate secretion & CG-resistent T-cells eradicate tumor.

CGs inhibit translation of MYC and activate MYC-repressed signaling pathways leading to recruitment and activation of T cells. In addition, CGs are efficient inhibitors of glycolysis, because treatment with CGs blocks the secretion of immunosuppressive lactate. However, conventionally, they also inhibit the metabolism of immune cells in the organism. The invention uncouples the effects of CGs on tumor cells and immune cells, thus enabling more effective immunotherapy.

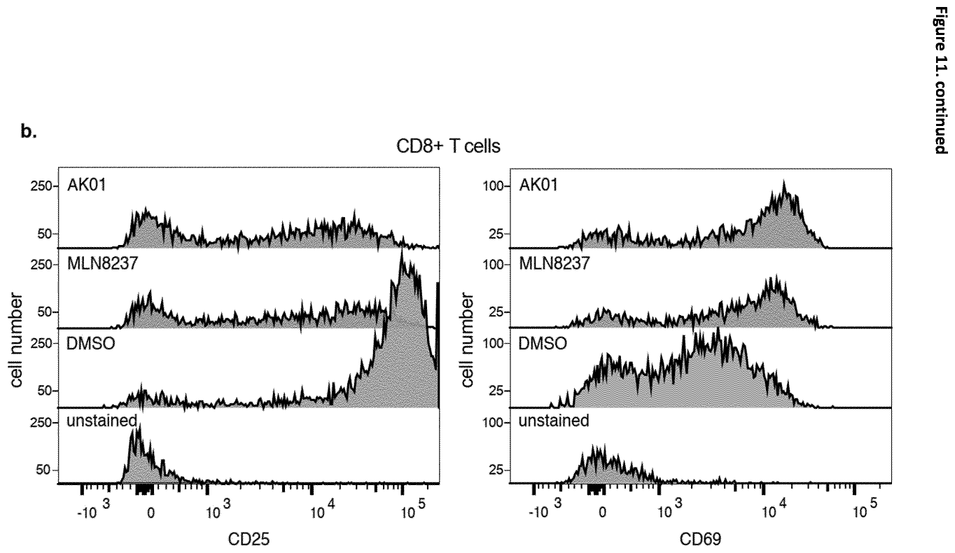

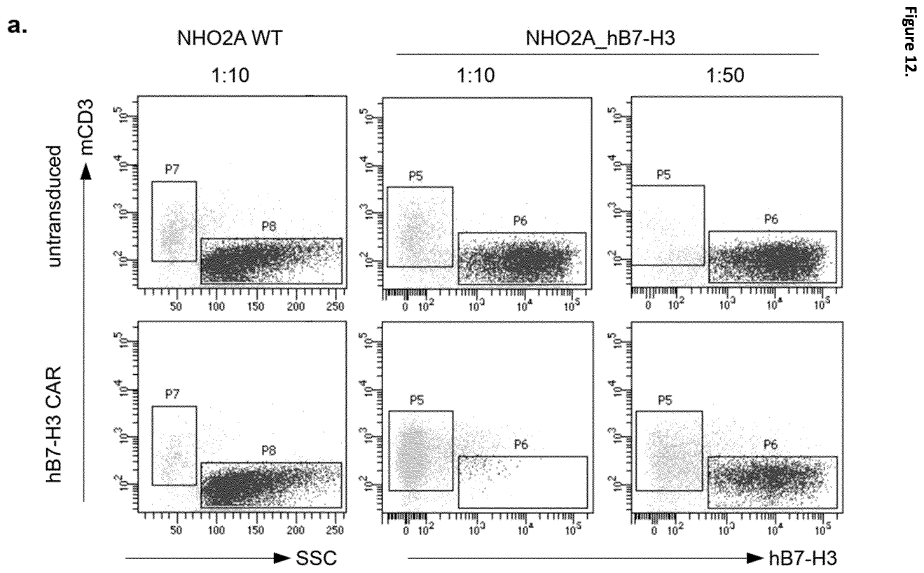

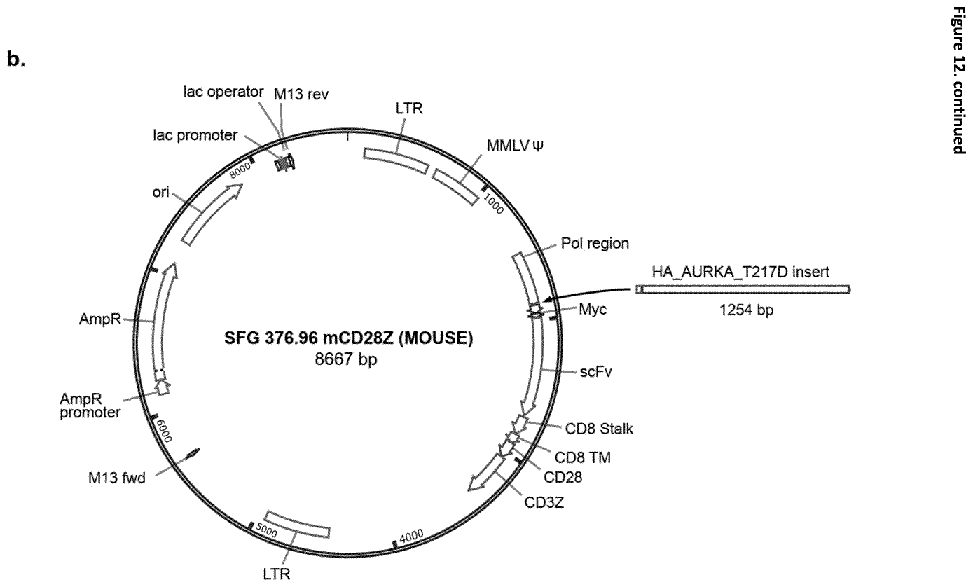

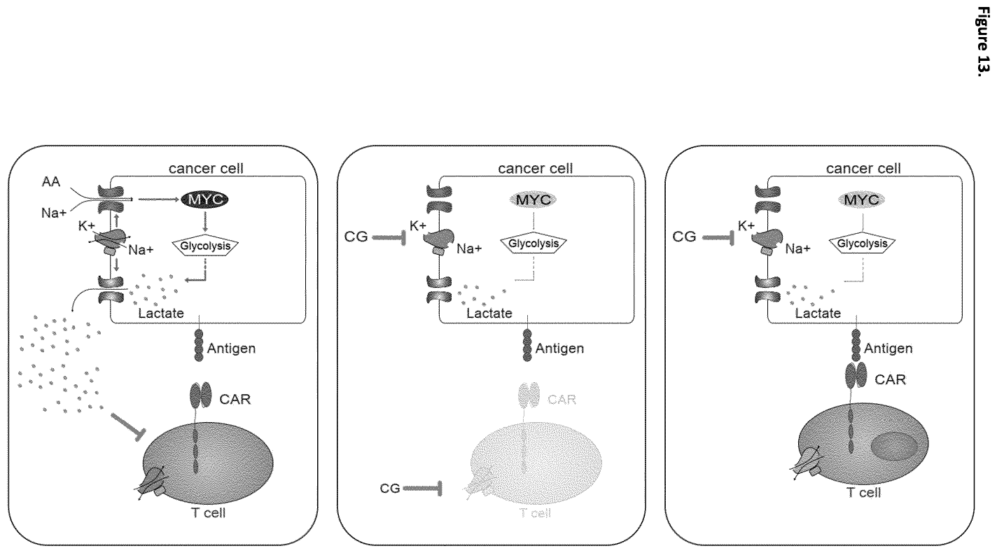

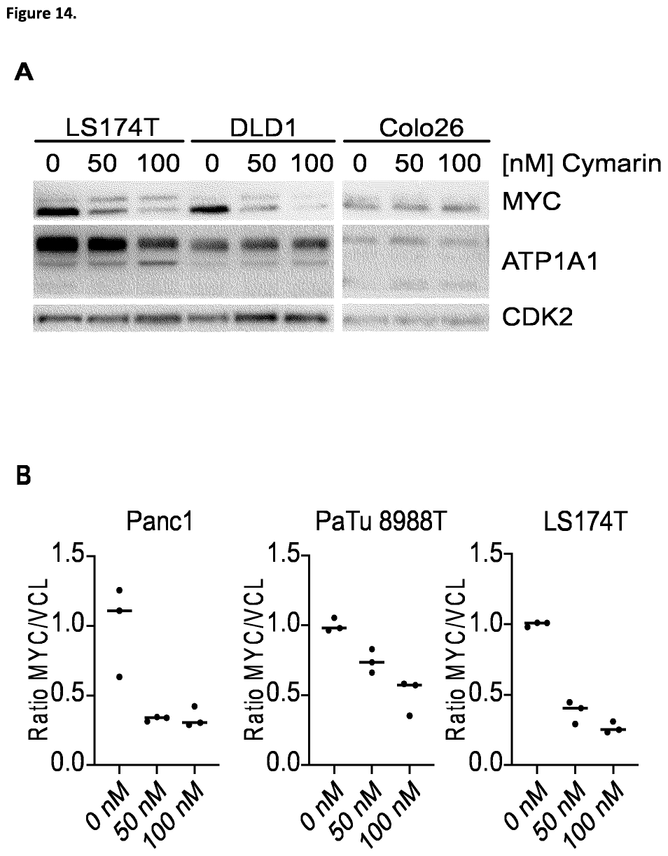

FIG. 14. CGs reduce the amount of MYC protein in human but not in murine tumor cells. A: Immunoblot of tumor cells after 24 h treatment with Cymarin. B: Quantification of MYC protein levels in human pancreatic tumor cells and colorectal tumor cells.

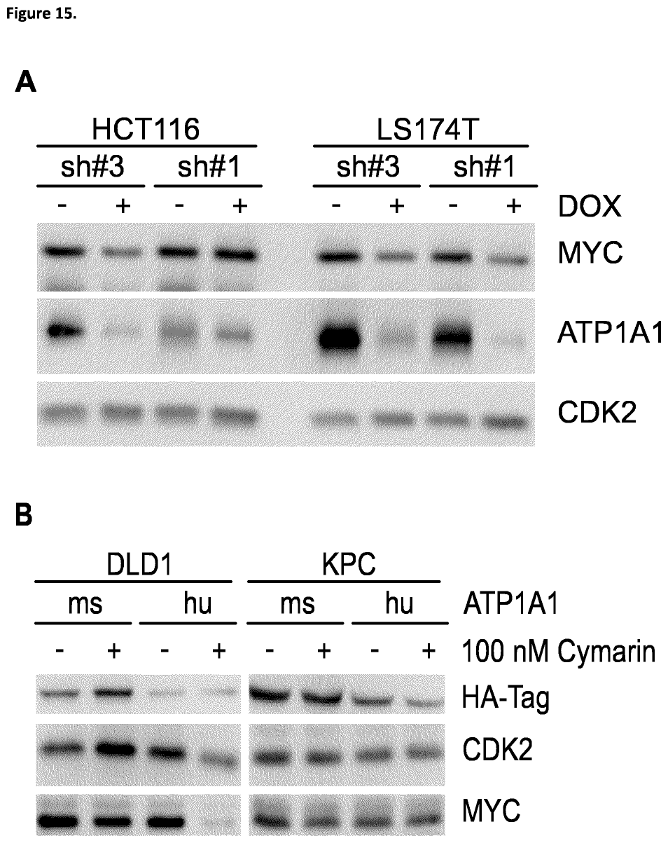

FIG. 15. The effect of CG on MYC protein levels is mediated by inhibition of the NA/K pump. A: Depletion of the ATP1A1 subunit of the NA/K pump leads to the reduction of MYC protein levels. B: Ectopic expression of murine ATP1A1 renders the expression of MYC insensitive from the addition of cymarin, a prototypical KG.

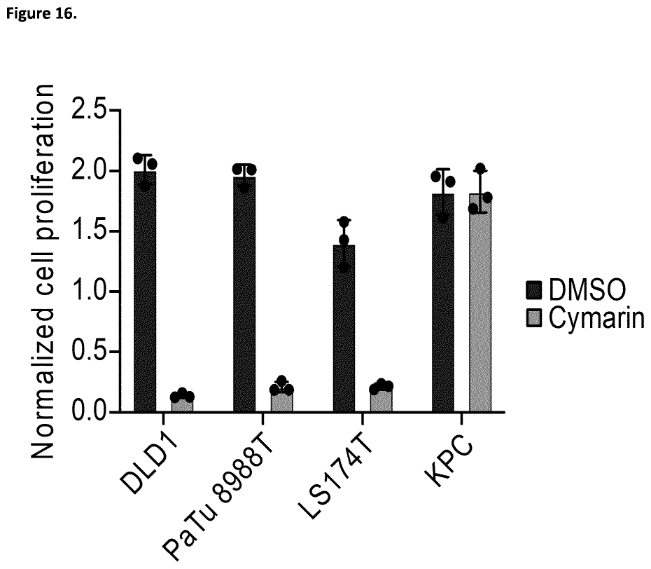

FIG. 16. Growth of human (DLD1, PaTu 8988T, Ls174T) and murine (KPC) tumor cells under control conditions (DMSO; left) and after treatment with cymarin (100 nM; right).

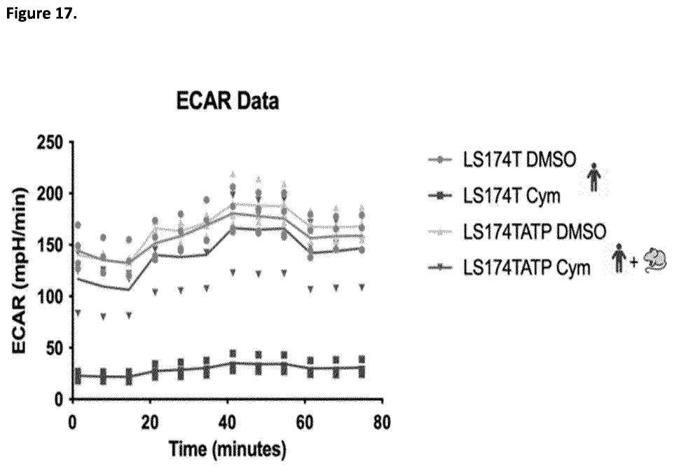

FIG. 17. The extracellular acidification rate (lactate secretion) in human LS174 colo-rectal tumor cells is significantly reduced by treatment with cymarin. This effect can be reduced by overexpression of the murine 1 isoform of Na⁺/K⁺-ATPase.

FIG. 18. The human and murine isoform of ATP1A1 share 97% homology. Inducing two mutations in a glutamine and an asparagine destroys binding site for CGs and makes the ATP1A1 resistant against the treatment with CGs.

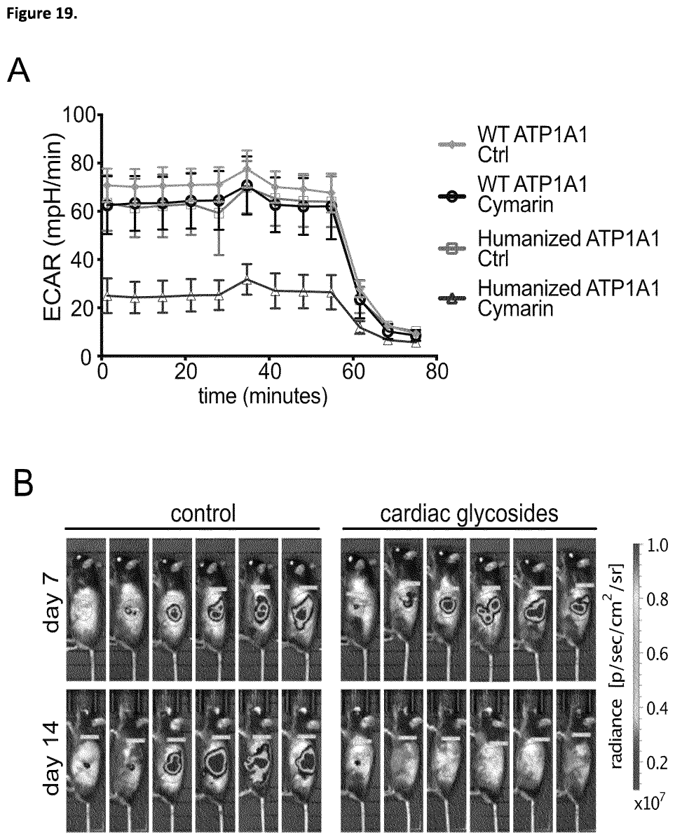

FIG. 19. A: Lactate secretion from pancreatic cancer cells with a humanized Na⁺/K⁺-ATPase (Clone2) and from control cells (Clone 1). B: Growth of PDAC tumors with a humanized Na⁺/K⁺-ATPase after treatment with KG. Cells express a luciferase gene and luciferase signal reflects tumor size.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Further, reference is made herein to the following amino acid sequences (and also to the respective coding nucleotide sequences):

>sp|P05023|AT1A1_HUMAN Sodium/potassium-transporting ATPase subunit alpha-1
OS = Homo sapiens OX = 9606 GN = ATP1A1 PE = 1 SV = 1
SEQ ID NO. 1
MGKGVGRDKYEPAAVSEQGDKKGKKGKKDRDMDELKKEVSMDDHKLSLDELHRKYGTDLS
RGLTSARAAEILARDGPNALTPPPTTPEWIKFCRQLFGGFSMLLWIGAILCFLAYSIQAA
TEEEPQNDNLYLGVVLSAVVIITGCFSYYQEAKSSKIMESFKNMVPQQALVIRNGEKMSI
NAEEVVVGDLVEVKGGDRIPADLRIISANGCKVDNSSLTGESEPQTRSPDFTNENPLETR
NIAFFSTNCVEGTARGIVVYTGDRTVMGRIATLASGLEGGQTPIAAEIEHFIHIITGVAV
FLGVSFFILSLILEYTWLEAVIFLIGIIVANVPEGLLATVTVCLTLTAKRMARKNCLVKN
LEAVETLGSTSTICSDKTGTLTQNRMTVAHMWFDNQIHEADTTENQSGVSFDKTSATWLA
LSRIAGLCNRAVFQANQENLPILKRAVAGDASESALLKCIELCCGSVKEMRERYAKIVEI
PFNSTNKYQLSIHKNPNTSEPQHLLVMKGAPERILDRCSSILLHGKEQPLDEELKDAFQN
AYLELGGLGERVLGFCHLFLPDEQFPEGFQFDTDDVNFPIDNLCFVGLISMIDPPRAAVP
DAVGKCRSAGIKVIMVTGDHPITAKAIAKGVGIISEGNETVEDIAARLNIPVSQVNPRDA
KACVVHGSDLKDMTSEQLDDILKYHTEIVFARTSPQQKLIIVEGCQRQGAIVAVTGDGVN
DSPALKKADIGVAMGIAGSDVSKQAADMILLDDNFASIVTGVEEGRLIFDNLKKSIAYTL
TSNIPEITPFLIFIIANIPLPLGTVTILCIDLGTDMVPAISLAYEQAESDIMKRQPRNPK
TDKLVNERLISMAYGQIGMIQALGGFFTYFVILAENGFLPIHLLGLRVDWDDRWINDVED
SYGQQWTYEQRKIVEFTCHTAFFVSIVVVQWADLVICKTRRNSVFQQGMKNKILIFGLFE
ETALAAFLSYCPGMGVALRMYPLKPTWWFCAFPYSLLIFVYDEVRKLIIRRRPGGWVEKE
TYY
>sp|Q8VDN2|AT1A1_MOUSE Sodium/potassium-transporting ATPase subunit alpha-1
OS = Mus musculus OX = 10090 GN = Atpla1 PE = 1 SV = 1
SEQ ID NO. 2
MGKGVGRDKYEPAAVSEHGDKKGKKAKKERDMDELKKEVSMDDHKLSLDELHRKYGTDLS
RGLTPARAAEILARDGPNALTPPPTTPEWVKFCRQLFGGFSMLLWIGAILCFLAYGIRSA
TEEEPPNDDLYLGVVLSAVVIITGCFSYYQEAKSSKIMESFKNMVPQQALVIRNGEKMSI
NAEDVVVGDLVEVKGGDRIPADLRIISANGCKVDNSSLTGESEPQTRSPDFTNENPLETR
NIAFFSTNCVEGTARGIVVYTGDRTVMGRIATLASGLEGGQTPIAEEIEHFIHLITGVAV
FLGVSFFILSLILEYTWLEAVIFLIGIIVANVPEGLLATVTVCLTLTAKRMARKNCLVKN
LEAVETLGSTSTICSDKTGTLTQNRMTVAHMWFDNQIHEADTTENQSGVSFDKTSATWFA
LSRIAGLCNRAVFQANQENLPILKRAVAGDASESALLKCIEVCCGSVMEMREKYSKIVEI
PFNSTNKYQLSIHKNPNASEPKHLLVMKGAPERILDRCSSILLHGKEQPLDEELKDAFQN
AYLELGGLGERVLGFCHLLLPDEQFPEGFQFDTDDVNFPVDNLCFVGLISMIDPPRAAVP
DAVGKCRSAGIKVIMVTGDHPITAKAIAKGVGIISEGNETVEDIAARLNIPVNQVNPRDA
KACVVHGSDLKDMTSEELDDILRYHTEIVFARTSPQQKLIIVEGCQRQGAIVAVIGDGVN
DSPALKKADIGVAMGIVGSDVSKQAADMILLDDNFASIVTGVEEGRLIFDNLKKSIAYTL
TSNIPEITPFLIFIIANIPLPLGTVTILCIDLGTDMVPAISLAYEQAESDIMKRQPRNPK
TDKLVNERLISMAYGQIGMIQALGGFFTYFVILAENGELPFHLLGIRETWDDRWVNDVED
SYGQQWTYEQRKIVEFTCHTAFFVSIVVVQWADLVICKTRRNSVFQQGMKNKILIFGLFE
ETALAAFLSYCPGMGAALRMYPLKPTWWFCAFPYSLLIFVYDEVRKLIIRRRPGGWVEKE
TYY

The invention will now be described by reference to the following examples which are merely illustrative and are not to be construed as a limitation of the scope of the present invention.

### Example 1

### Materials and Methods (Especially Pertaining to Examples 2 to 7)

Generation of a Pool of Cells with a Mutagenized Target Protein

The generation of (a pool of) cells with a mutagenized target protein, the target identification (in particular the identification of targets of (small molecule) DDIAs) and/or the scanning of essential genes is, for example, performed by using CRISPR-Cas mutogenesis. This is, for example, described in Neggers (Nat Commun 9(1), 2018, 502).

Screening of Libraries, e.g. Lentiviral Libraries

The screening of libraries, e.g. lentiviral libraries is, for example, performed by using pooled sh RNA and, CRISPR screens. This is, for example disclosed in Cluse (Methods Mol Biol 1725, 2018, 201-227).

### Transduction of T-Cells or Other Cells Described Herein

For gene transfer, retroviral transduction is, for example, used, or the sleeping beauty transperson technology (e.g. as described in Monjezi, Leukemia 31, 2017, 186-94). The transduction of T-cells is, for example, performed as a lentiviral transduction; this is, for example, Prommersberger (Current Protocols in Immunology, 128, 2020, e93).

Stimulation of T-Cells, or Other Cells Described Herein, and Measurement of their Functionality

The stimulation of (T)-cells and measurement of their functionality is, for example, performed as in Reinwald (The Journal of Immunology 180 (9), 2008, 5890-5897).

### Measurements of (Immune) Cell Proliferation

The measurements of (immune) cell proliferation (e.g. lymphocyte proliferation) is, for example, performed by using a fluorescent dye, in particular an intracellular fluorescent dye (e.g. carboxyfluorescein diacetate succinimidyl ester). This is, for example, described in Quah (Nat Protoc 2, 2007, 2049-2056).

### Screening of Cells/Cell Lines for Drug Sensitivity

The screening of cells/cell lines for (anti-cancer drug (DDIA)) sensitivity, in particular of tumor/cancer cells/cell lines, is, for example, performed as in Barretina (Nature 483(7391), 2012, 603-607).

### Model Systems

### Pancreas Ductal Adenocarcinoma (PDAC)

The pancreas model of KPC tumors, i.e. KPC cells, is an orthotopic transplant model, for which extensive experimental experience exists and multiple transplant experiments can routinely be carried out. The genotype of the cells is as follows: Pft1a/Cre; Kras+/LSL-G12D; p53loxP/R172H (Hingorani, Cancer cell 7, 2005, 469-83). While in the work up to now this single cell line was used, the spectrum is now broadened, and other cell lines are brought in.

### (Metastatic) Colorectal Carcinoma (CRC)

Cultured (human or mouse) colon cancer cells or (human or mouse) colon cancer organoids are used as cellular models of CRC (e.g. as described herein elsewhere).

### Liver Metastases of CRC

For metastatic tumors, particularly those with liver metastases, a two-step surgical procedure has been proposed to prevent postoperative liver failure (Lang loc.cit.). In the first step a small part of the liver is cleaned from metastases and portal blood flow to the larger, non-cleaned lobe is cut. While this “cured” section regenerates, the other lobe partially contributes to sustain sufficient liver function. When the “cured” lobe reaches a functionally sufficient volume the still metastases carrying part can be removed, but tumor progression in the tumor-bearing lobe can cause unresectability. When combined with this surgical strategy, any molecular strategy that suppresses the growth of colon metastases in one half while allowing liver regeneration even for a limited time period therefore has the potential to cure a significant fraction of patients. mouse model has been established that mimics this clinical situation. In particular, a syngeneic model with metastatic murine cell line has been used as established. This model carries KRAS, p53, APC and TGFbeta receptor mutations (Tauriello, Nature 554, 2018, 538-43). As for the PDAC model, additional cell lines are obtained to validate the findings. The metastatic murine cell line is transplanted either directly into the liver (cf. FIG. 8b), or injected into the spleen, leading to multifocal colonization of multiple lobes (cf. FIG. 8b). This has been combined with liver resection and the subsequent regeneration has been quantified (cf. FIG. 8c). Further, this model can now be used to measure growth of metastases during liver regeneration following resection.

### Material and Methods (Especially Pertaining to Example 8)

### Cells and Transfection

The murine neuroblastoma cell line NHO2A was cultivated in RPMI1640 supplemented with 10% FCS, penicillin and streptomycin. Analogous to the already published human Aurora-A mutant (T217D), the murine Aurora-A mutant (T208D) was constructed. (murine) Aurora-A^{wt} and (murine) Aurora-A^{T208D} were cloned into the lentiviral pRRL overexpression vector. This vector was used to stably overexpress Aurora-A in the murine neuroblastoma cell line NHO2A.

### Proliferation Assay

T cells were isolated using the Pan T cell isolation kit (Miltenyi Biotec) according to manufacturer's recommendations. Cells were stained with a proliferation dye (Thermo Fisher Scientific) and activated with α-CD3 (0.75 mg/ml, bound to the plate) and α-CD28 (1 mg/ml, dissolved) (Invitrogen). For each test unit of the plate, 3×10⁵ cells in 200 μl were needed. Proliferation was measured after 96 h. Cells were seeded in IMDM GlutaMAX™ (Thermo Fisher Scientific) supplemented with 10% FCS, penicillin and streptomycin, 0.1 mM 2(B)-mercaptoethanol (Gibco) and 10 ng/ml IL-7 (PeproTech).

### Designing CAR T Cells

SFG-gammaretroviral vector (RRID: Addgene_22493) was used for designing CAR T cells. The anti B7H3 CAR-T was synthesized within SFG and contains the following components: IL-2 signal peptide, the single chain variable fragment TE9 ScFv (376.96 B7-H3 antibody), CD8 hinge and transmembrane, co-stimulatory CD28 endodomain, and intracellular signaling domain CD3zeta. The construct was a kind gift of John Anderson. (murine) Aurora-A^{wt} and (murine) Aurora-A^{T208D} was designed to be included into the construct.

### Generation of Retroviral Supernatant and Transduction of Murine CD4+ and CD8+ T-Cells

One day prior to transfection 2×10⁶ ecotrophic phoenix cells were seeded on 10 cm Nunc plates in DMEM supplemented with 10% FCS, penicillin and streptomycin. Shortly before transfection medium was changed to 2% FCS containing media and cells were transfected with retroviral plasmid and Genejuice transfection agent (Merck). Retroviral supernatant was harvested 48 and 72 hours after transfection. Isolated T-cells were activated and seeded on 12-well plates one day prior to transduction. 80% of media from T-cells was carefully removed and 1-3 ml of retroviral supernatant supplemented with 10 g/ml Polybrene was added to the T-cells. Transduction was performed by spinoculation at 1,500×g and 32° C. for 90 min. Retrovirus was replaced 2-4 hours after spinoculation with full T-cell media. Transduction efficiency and viability were measured using flow cytometry 24 hours post transduction.

### Analysis of CAR T-Cell Effector Function In Vitro

To analyze the effect, CAR T-cells were co-cultivated with different effector target ratios (E:T) with NHO2A cells overexpressing a truncated hB7-H3 construct. For the cocultivation assay, 5×10⁴ tumor cells were seeded in 24-well plates and left to settle for 2-3 hours. Infected T-cells were harvested, media from tumor cells was aspirated and T-cells were cultivated onto tumor cells in IMDM supplemented with 10% FCS, penicillin/streptomycin and 2(B)-mercaptoethanol. Coculture was harvested with trypsin after 72 hours and T-cells were stained for α-CD3 and tumor cells for α-hB7-H3. Wildtype NHO2A cells were distinguished from T-cells by size.

### Material and Methods (Especially Pertaining to Example 9)

### Immunoblot

After treatment for 24 h, cells were harvested in RIPA buffer containing protease and phosphatase inhibitor cocktails. The protein concentration was determined using BCA or Bradford assay. 15 μg of protein was loaded on a 10% SDS gel. After electrophoresis, proteins were transferred to 0.45 μm PVDF membrane and incubated with primary antibodies at 4° C. overnight. The signal was detected using peroxidase-conjugated species-specific secondary antibodies and visualized at LAS-4000 Luminescent Image Analyzer (Fujifilm).

### Proliferation Measurement

For the proliferation measurement cells were treated for 96 h with 100 nM of Cymarin or DMSO respectively and then counted using CASY cell counter.

### Measurement of Extracellular Fluxes Using Seahorse XF96

20.000 cells per well were seeded in a XF 96-well cell culture microplate in 80 ml of culture medium, and incubated overnight at 37° C. and 5% CO₂. The culture medium was replaced with 180 ml of bicarbonate-free RPMI and cells were incubated at 37° C. for 30 minutes before the measurement. The oxygen consumption rates (OCR) were measured using an XF96 Extracellular Flux Analyzer (Agilent), first with no additions, then after addition of oligomycin (1 μM), carbonyl cyanide 4-(trifluoromethoxy) phenylhydrazone (FCCP, 0.5 μM) and rotenone+antimycin A (1 μM). After the measurement the values were normalized on the total protein concentration per well, determined by BCA assay.

### Cardiac Glycosides

Cymarin Sigma; Ouabain Sigma; Digitoxin Sigma

### Amino Acid Sequences

murine Na⁺/K⁺-ATPase (SEQ ID NO.2; see also FIG. 18 (relevant part of SEQ ID NO.2)); human Na⁺/K⁺-ATPase (SEQ ID NO.1; see also FIG. 18 (relevant part of SEQ ID NO.1))

### Example 2: Targeting Resolution of TRCs in PDAC and CRC

Targeting the mechanisms that resolve TRCs is considered a viable strategy to inflict tumor cell-specific DNA damage and/or to sensitize tumors to immune cell-mediated killing. Several of the mechanisms described herein appear to be specific for MYCN-driven tumors. For example, (c-)MYC (herein also just “MYC”) binds only very weakly to Aurora-A (Dauch, Nat Med 22, 2016, 744-53). Therefore, pathways have been identified that resolve TRCs in models of MYC driven tumors, which encompasses the vast majority of all tumors. Specifically, there was a focus on conflicts in a cell line established from the KPC model of pancreas carcinoma (Hingorani, loc.cit.). This reflects human PDAC. In this model, an siRNA screen of 86 candidate genes was performed using several parallel microscope-based assays to measure the occurrence of TRCs (FIG. 1).

In order to determine whether the pathways are conserved among murine and human tumor cells, and to define precisely the critical components in colon cancer cells, these screens were also performed in cultured human colon cancer cells. Some results were further validated in colon cancer organoids. These screens yielded a virtually identical, but slightly broader hit list (FIG. 2). While these assays (and further analysis to be performed) confirm the relevance of the MYCN-driven pathways described before, the strongest hits of the screens define three distinct molecular mechanisms:

First, PAF1c complexes are required to resolve TRCs, consistent with their role in yeast (Poli, Genes & development 30, 2016, 337-54). In mammalian cells, they are recruited to core promoters by the MYC oncoprotein (Endres, Mol Cell S1097-2765(20), 2021, 30956-4; Jaenicke, Mol Cell 61, 2016, 54-67). Recruitment of PAF1 to promoters has two functions: PAF1c recruits the double-strand break repair machinery via the ubiquitin-ligases RNF20 and RNF40 (Endres loc.cit.), but this pathway is not critical for resolving TRCs. Rather, cyclinK/CDK12 complexes, which are recruited by PAF1c to promoters (Yu, Science 350, 2015, 1383-6) are downstream of PAF1c in the replication pathway (Gaballa, unpublished).

Second, splicing and termination complexes are also critical for resolving TRCs, since the screens in both models (PDAC and CRC) identify components of the splicing machinery. A close relationship between defects in splicing and replication stress has been noted before in myelodysplastic syndromes and is thought to reflect the accumulation of R-loops due to inefficient mRNA processing (Chen, Mol Cell 69, 2018, 412-25).

Third, chromatin association of the PNUTS/PP11 phosphatase complex is controlled by the NUAK1/ARK5 kinase that has been characterized as a synthetic lethal interaction with MYC (Cossa loc. cit.; Liu, Nature 483, 2012, 608-12). PNUTS/PP1 has emerged as an essential regulator of replication conflicts (Landsverk, Cell reports 33, 2020, 108469; Landsverk, Nucleic Acids Res 47, 2019, 1797-1813); and these findings have been confirmed and are extended for NUAK1.

Based on the similarity to the results obtained in MYCN-driven tumors, the common denominator of these three pathways is considered to be that they are all required to suppress the accumulation of R-loops during S-phase. Importantly, each pathway contains components that are druggable with molecules that are either currently available or in development. This allows to confirm the therapeutic validity of this consideration by using small molecule inhibitors.

### Example 3: Induction of TRCs by Small Molecules (Small Molecules DDIAs)

The functional analyses described above, as well as the additional work on an inhibitor of RNA Polymerase I transcription described below, identify four different strategies to induce TRCs that can be realized with currently available small molecules (to be used as the DDIAs). Besides the mechanistical studies these can be used in vivo in mouse tumor models, and they are, in part, already in human use.

First, SR4835 is a specific and potent inhibitor of the CDK12 kinase (cf. FIG. 1; also described in Quereda, Cancer cell 36, 2019, 545-58). It can be combined with inhibitors of the ATR or ATM kinases. Available data suggest that this inhibitor has low toxicity. Additional CDK12 inhibitors that covalently modify a specific cysteine in CDK12 are in development (Zhang, Nat Chem Biol 12, 2016, 876-84).

Second, inhibitors of mRNA splicing are available that can be used in mice and are in clinical trials (Bowling, Cell 184, 2021, 384-403). In addition, a previously characterized anti-tumor sulfonamide was found to suppress tumor growth since it acts as a “glue” molecule that links a splicing factor, RBM39, with an ubiquitin-ligase, causing degradation of RBM39 (Han, Science 356, 2017). Furthermore, an inhibitor of the polyadenylation complex CPSF3 is in development and may, for example, be used for proof-of-principle experiments (Kakegawa, Biochem Biophys Res Commun 518, 2019, 32-7).

Third, the work on NUAK1 resulted in a specific inhibitor established with Bayer (BAY-880) (Cossa, Mol Cell 77, 2020, 1322-39). Several other NUAK1 inhibitors that can be used in preclinical models are currently available.

Fourth, Work described below shows that an available inhibitor of the SL1 initiation complex for RNA Polymerase I that transcribes ribosomal RNA genes, CX-5461 (Drygin, Cancer research 71, 2011, 1418-30), induces TRCs.

In addition to the data described above for the CDK12 inhibitor (FIG. 1), further data which indicate that TRCs can be triggered have been obtained. In this context, the following strategies have been applied:

### Small-Molecule Inhibitors of the NUAK1 Kinase

NUAK1/ARK5 was identified as a kinase which is required for the survival of cells with deregulated MYC expression (Liu, Nature 483, 2012, 608-12). While initial data suggested a role for NUAK1 in the cytosol, the recent findings show that NUAK1 almost exclusively localizes in the nucleus. Indeed, it was found that NUAK1 regulates early steps of transcription since it phosphorylates PNUTS, a regulatory subunit of nuclear protein phosphatase 1 (PP1) complexes (Cossa loc.cit.). The critical phosphorylation site (S313) has been identified, a phospho-specific antibody has been raised and it was shown that it can be used as a reliable marker for NUAK1 activity in vivo (FIG. 3a). Further, it was shown that phosphorylation by NUAK1 is required for chromatin association of PNUTS (FIG. 3b). The PNUTS/PP1 complex is a phosphatase for Ser5 of the carboxy-terminal repeat of RNAPII. Phosphorylation at this site is required for RNAPII to clear the promoter, suggesting that dephosphorylation by PNUTS/PP1 promotes termination close to the promoter. Recent observations demonstrate that PNUTS/PP1-mediated early termination is required to co-ordinate transcription with DNA replication (Landsverk, 2020, loc.cit.; Landsverk, 2019, loc.cit.).

Since phosphorylation by NUAK1 is required for chromatin association of PNUTS/PP1, this indicates that inhibition of NUAK1 can trigger TRCs. It has further been validated that inhibition of NUAK1 causes conflicts between RNA Polymerase II and DNA replication forks (FIG. 3c) and suppresses the proliferation of pancreas cells (data not shown) and colon cancer organoids in co-operation with inhibition with low concentration of ATR (FIG. 3d). Importantly, several NUAK1 inhibitors were used to validate growth suppression of organoids in conjunction with ATR, minimizing the danger of an off-target activity (FIG. 3d). In sum, inhibition of NUAK1 emerges as a valid strategy to trigger TRCs.

### TRCs Induced by Inhibition of RNA Polymerase I

Deregulation of the WNT pathway in CRC not only deregulates transcription by RNAPII, but also by RNA polymerase I, which transcribes ribosomal RNAs in the nucleolus; this leads to enhanced synthesis of ribosomal RNA (Morral, Cell stem cell 26, 2020, 845-61). This is mediated by a beta-catenin dependent upregulation of multiple components of the ribosomal RNA synthesis machinery (FIG. 4a). Further to these observations, the effect of CX-5461, an inhibitor of RNA polymerase I transcription on the growth of CRC metastases, was analyzed. CX-5461 does not inhibit binding of RNA polymerase I to its promoters, but rather “freezes” it at the promoter and prevents the transition of RNA polymerase I into active elongation (Mars, NAR Cancer 2, 2029, zcaa032). This observation was confirmed. Indeed, CX-5461 causes massive DNA damage during DNA replication in conjunction with ATR inhibition (FIG. 4b) and induces conflicts with DNA replication as measured by an increased proximity between RPA194, a subunit of RNA polymerase I, and RAD9, a marker for stalling replication forks (FIG. 4c). Most importantly, combining low concentrations of CX-5461 and an ATR inhibitor almost completely suppressed colony formation of several human CRC cell lines in a p53-independent manner (FIG. 4d) as well as of murine CRC organoids (FIG. 4e). Since there is an ongoing debate on the primary mechanism of CX-5461 action (Bruno, PNAS 117, 2020, 4053-60; Sanij, Nat Commun 11, 2020, 2641; Xu, Nat Commun 8, 2017, 14432), genetic work to validate its precise mechanism of action, and to further confirm that combining POL1 and ATR inhibition is a valid strategy to induce TRCs, is performed.

### Example 4: TCRs can Trigger T Cell-Dependent Killing In Vivo

TRCs and the ensuing DNA damage not only trigger tumor cell-intrinsic responses, but can also stimulate T cells to recognize and kill tumor cells. Evidence for this comes from the observation that tumor regression of MYCN-driven neuroblastoma cells upon treatment with Aurora-A/ATR inhibitors is paralleled by activation of the STING pathway and a massive infiltration of immune cells. Transplantation experiments of tumor cells into different immune-compromised mice documents that the therapeutic efficacy of the treatment depends on T cells (Roeschert loc.cit.). Furthermore, expression of an inducible shRNA targeting endogenous MYC in KPC cells recapitulates virtually all expected features described for MYC depletion in culture (FIG. 5). For example, MYC is required for the rapid growth of KPC cells. As compared to cell culture, however, tumors in vivo grow about 100-fold more slowly and MYC is not required for tumor growth in vivo per se. Rather, the dependence on MYC in vivo is dependent on an intact immune compartment: there is often a complete tumor regression when MYC is depleted upon transplantation in syngeneic mice, while tumors grow almost unimpaired upon MYC depletion in tumors transplanted into the most immune-compromised mice (NRG). As in the neuroblastoma model, T cells are the key effector cells for regression. The subsequent mechanistic analyses show unequivocally that MYC is stringently required for preventing TRCs in these cells since it is upstream of the PAF1/CDK12 pathway described above. This supports a model in which both processes are linked and indicates that cells with DNA damage resulting from unresolved TRCs are eliminated by the immune system. In this model, MYC suppresses TRCs not only to promote cell cycle progression but also to escape recognition of damaged cells by the immune system.

Parallel to these mechanistic analyses, it was explored whether these findings can be exploited for the development of future tumor therapies. Specifically, it was tested whether PDAC cells can be sensitized to an attack by CAR T cells. Since no CAR T cells are available that recognize KPC cells, a human antigen, ROR1, against which well-characterized CART cells, which currently entered clinical trials, are available (Hudecek, loc.cit.; Wallstabe, loc.cit.), was expressed on the tumor cells. B7H3 may analogously be used in this respect. The experimental system that was set up is shown in FIG. 6. Briefly, in in vitro experiments, KPC cells expressing human ROR1 were rapidly eliminated by murine T cells engineered to express a CAR against human ROR1, but not by control T cells. Intriguingly, transplantation of CAR T cells into mice which carry a KPC tumor expressing human ROR1 had absolutely no effect on tumor growth and survival; this is considered to reflect the immune-evasive properties of these tumors. This situation changed drastically when MYC is depleted by doxycycline-mediated induction of shRNA; this causes a significant expansion of life-span by itself, but transplantation of T cells or, even more, CAR T cells drastically expands life span, with a fraction of mice remaining tumor-free for a prolonged time. Of note: normal T cells may recognize human ROR1 in this model, so the effect of naïve cells may be overestimated. These data show that depletion of MYC can break the immune escape mechanisms of these tumors and provide the proof-of-principle that this can now be used to benchmark all targeted strategies.

### Example 5: Triggering TRCs In Vivo

TRCs are triggered by using small molecules (as DDIAs) in vivo, the responses of PDAC tumors and CRC metastases to these treatments are determined using the experimental systems outlined above and the relative contributions of tumor cell intrinsic and immune cell mediated responses are determined. In this context, four chemical strategies are explored (see also Example 3, supra):
NUAK1/ATR inhibition
Spliceosome/ATR inhibition
POL1/ATR inhibition
CDK12/ATR inhibition

In practical terms, two major aspects are addressed to carry out these experiments:

First, the dosing schedule and in vivo activities are well established for almost all compounds. This is also done for the NUAK1 inhibitors. Currently, three NUAK1 inhibitors are available and the identification of a reliable biomarker (pS303 phosphorylation) enables to measure their in vivo efficacy. Mass spectrometry methods have also been established that can measure the stability of a compound in vivo and concentrations in the target tissue. Together, this allows to establish the necessary schedule. In this context, the required amounts of the inhibitors to be used are also synthesized; and the same methods allow to perform quality control and purity checks.

Second, the correct combinations with inhibitors of checkpoint are optimized. While work up to now has been performed with inhibitors of the ATR kinase, ATR is activated specifically in response to head-on conflicts. In contrast, co-directional conflicts, which occur because the replication fork moves faster than RNA polymerases, activate ATM (Hamperl, Cell 170, 2017, 774-86). Thus, at least some of the strategies provided herein are considered to be even more potent when combined with ATM inhibition. This is determined in tissue culture, followed by the relevant in vivo work.

### Tumor Growth and Survival

Both, the KPC cells (used to model human PDAC) and the CRC cells/aganoids that are used in these experiments, are labeled with luciferase and the imaging technologies are established, allowing to monitor tumor growth in a longitudinal and non-invasive manner. Survival curves have been established and extensively characterized in the PDAC model and are also established in the metastatic model. The use of a transplant model allows to use transplant tumor cells in different strains of host mice. As described above, the contribution of the host immune system to responses in PDAC cells have already been established; and similar experiments are carried out to assess the contribution of the host immune system to therapeutic responses for liver metastases (resulting from CRC).

### Develop Assays for Validating On-Target Activity

On target activity assays are available for all inhibitors used. For both, ATR kinase and CDK12, specific phospho-antibodies are commercially available, and these tools have been generated also for NUAK1. Inhibition of the spliceosome can be measured by next generation sequencing, in particular in combination with 4sU-labeling in tissue culture. This information is used to identify introns for which appropriate PCR primers can be used to assess effects of spliceosome inhibitors in tissue samples recovered from tumors. Similarly, the rate of rRNA synthesis can be estimated using primers that cover an intron in the pre-rRNA that is rapidly spliced out after synthesis. This can be sued to measure the effect of POL1 inhibitors in vivo. Assays to document the occurrence of TRCs are available, and are further expanded.

From the work on the neuroblastoma model, it is deduced that double-strand break formation occurs in a highly tumor-specific manner. This can be assessed by standard markers (y-H2AX and pKAP1), comparing tumor tissue to highly proliferative normal tissue, like the transit amplifying cells in the gut or hematopoietic (stem) cells in the bone marrow. The required direct sequencing technologies (BLISS sequencing) for double-strand breaks have also been established (Endres loc.cit.; Yan, Nat Commun 8, 2017, 15058). It has also been shown that a monoclonal antibody used to detect R-loops (S9.6) can be used in histology. This is validated using appropriate controls. In addition to that, the staining is established with a recombinant, fluorescently-labeled protein encompassing the RNA/DNA hybrid-binding domain of RNAseH1. This is a valid detection reagent for R-loops in histological sections. Direct evidence for TRCs is obtained by proximity ligation assays using antibodies for RNA Polymerases I and II on the one hand, and for either PCNA, which marks unstressed replication forks, or RAD1/9, which marks staling forks. These assays are established and validated for tissue culture experiments and it is explored whether these can be used in vivo.

### Immune Cell Involvement

Immune-competent (syngeneic) models are used to assess contribution of immune cells; histology is established for Tcells (and major subpopulation), B-cells, macrophages and NK cells. FACS-based assays has been established for several more immune cell markers (e.g. FoxP1 to detect regulatory immune cells).

### Example 6: Functional Screens In Vivo

Genetic screens and mechanistic analyses are performed to determine the precise relationship between triggering TRCs and immune cell-mediated killing of tumor cells and to understand how these can be improved.

The findings (i) that triggering TRCs in a model of murine neuroblastoma causes T-cell dependent tumor eradication (Roeschert loc.cit.); and (ii) that depletion of MYC in PDACs has little effect on tumor growth in vivo, but also causes both, TRCs and T cell-mediated tumor eradication, indicate that there is a mechanistic link between both processes. Without being bound by theory, this could be explained as follows: First, stalling of DNA polymerases generates single-stranded DNA that is recognized in the cytosol by the STING pathway leading to signaling to the immune system (Coquel, Nature 557, 2018, 57-61). Second, it has been shown that the deregulated transcription in PDAC cells lead to the accumulation of intron-derived double-stranded RNA that is exported from cells and recognized by a TLR3-dependent signaling pathway in a paracrine manner (Krenz loc. cit.). Further, recent work has demonstrated a similar pathway downstream of inhibitors of the spliceosome (Bowling, Cell 184, 2021, 384-403).

It is explored if, or which of, these mechanisms operate, and how tumors can be sensitized best to immune cell mediated killing by targeting these mechanisms.

Use is made of in vivo functional screening technologies that have been established for the PDAC model and is establish also for the CRC model. In the PDAC model, approximately 50,000 cells can be injected, which translates into about 500 sgRNAs corresponding to about 100 genes that can be analyzed in a single experiment. This is sufficient to screen very focused libraries and pilot screens have successfully been completed. In particular, two groups of genes are screened:

The first group of genes is based on the recent identification of the MYC and MYCN protein interactomes (Baluapuri, Mol Cell 77, 2019, 1322-39; Buchel, Cell reports 21, 2017, 3483-97). Comparison with interactomes identified in other laboratories identifies a consensus interactome of MYC proteins that can been screened in a single library (Baluapuri loc.cit.). It was also shown (see above) that the major function of endogenous MYC in PDAC is to enable tumor cells to escape from the immune system. Together, these findings indicate that some complexes of MYC and MYCN are critical, either directly or indirectly, to prevent TRCs and enables tumor cells to escape T cell-mediated immune surveillance. With this library, it is directly searched for these complexes.

The second group of genes is based on the concept that aberrant nucleic acid species, such as cytosolic ssDNA or dsRNA, mediate the sensitization of immune cell-mediated killing. Innate immune and dsRNA processing pathways are surveyed.

For assay conditions, there is specifically less interest in any complex or gene that is required for growth of KPC cells and of CRC metastases in immune-compromised hosts. This is because it is assumed that these are probably essential genes. To address also this, recipient cells are transplanted into NRG mice and every target that is depleted here is discarded. In contrast, there is particular interest in shRNAs that are specifically depleted under the following conditions:
In the presence, but not in the absence, of low concentrations of an ATR inhibitor.
In syngeneic mice, but not in NRG mice, in the absence or presence of low concentrations of an ATR inhibitor.
In the presence of CAR T-cells targeting a human antigen (e.g. ROR1 or B7H3), but not in the absence of CAR T-cells.

Any hits are followed up by mechanistic experiments to decipher the precise mechanism of action and by experiments to find ways to mimic the effects with small molecules (as the respective DDIAs).

### Example 7: Hybrid Tumor/T Cell Strategies

Hybrid targeting strategies are developed that exploit the fact that DNA damage is inflicted by inhibiting defined cellular targets. This enables to confer resistance to T cells and CAR T cells (and to other (immune) cells disclosed herein) to the treatment used; and to confirm that this enhances T-cell-mediated immune responses. The specific strategy has three elements (FIG. 7a):

First, the system that has already been established is exploited, in which human ROR1 (or another human antigen (e.g. B7H3)) is expressed in tumor cells, and a ROR1-specific CAR is expressed in murine T-cells. This renders the experiment independent of any specific CAR T-cell construct that in itself may be more or less functional and makes results comparable.

Second, the fact that both, PDAC and CRC cells, are susceptible to inhibition of Aurora-A kinase is used, and a base-line how the tumor models used respond to treatment with the most up-to-date inhibitor, LY3295668 (Gong, Cancer Discov 9, 2019, 248-63), is established.

Third, the fact that there are two Aurora-A alleles, T217D and T217E, which have been demonstrated to be resistant against available Aurora-A inhibitors is exploited (Sloane loc.cit.). As has been previously shown, this renders cells resistant against Alisertib and Alisertib-based PROTACs (Adhikari, Nat Chem Biol 16, 2020, 1179-88; Brockmann, Cancer cell 24, 2013, 75-89). It has now been demonstrated that this renders the kinase activity resistant against LY3295668 as well, which is the Aurora-A inhibitor that is currently moving into the clinic (FIG. 7b) (Gong loc.cit.).

Further, T-cells depend on Aurora-A for receptor signaling and proliferation (Blas-Rus, Nat Commun 7, 2016, 11389; Bustos-Moran, Scientific reports 9, 2019, 2211). The Aurora-A T217D and T217E alleles are transferred into CAR T-cells and their cytotoxic effects, cytokine secretion and proliferation are measured. These alleles are transferred into ROR1 (or, e.g. B7H3) CAR T-cells, and the experiments shown above are repeated in the presence and absence of LY3295668 with appropriate controls. For gene transfer, either retroviral transduction is used or the sleeping beauty-based transposon technology developed in the Danhof/Hudecek laboratory (Monjezi, loc.cit.). The evaluation of the experiment uses the parameters described before, measures T-cell infiltration, tumor growth by luciferase and survival. Similar experiments are performed in the CRC model.

These experiments for indicating that such hybrid strategies enhance the efficacy of T cell-based immune therapies are expanded to all inhibitors used in the assays. Specifically, high-throughput CRISPR-based mutagenesis screens have recently become available to inflict tiling mutants or point mutants at high frequency into any target gene of choice (Cuella-Martin, Cell 184, 2021, 1081-97; He, Nat Commun 10, 2019, 4541; Neggers loc.cit.; Cluse loc.cit.). Such screens are used in T-cell lines to select resistance alleles for the targets of all inhibitors used. In this context, cells are infected with large pools of lentiviruses that express collections of sgRNAs that cause point mutations or small deletions, resistant cells are recovered and the sequences of the target genes in the growing cells are recovered. The deconvolution of such high throughput screens has been established. The mutated alleles are subsequently be reintroduced into naïve cells and tested for their ability to confer drug resistance to CAR T-cells. Any positive allele are then be used in conjunction with the appropriate drug or drug combination to determine which allele enhances CAR T-cell efficacy most potently.

### Example 8

An inhibitor-resistant allele of Aurora-A was used (murine Aurora-A^{T208D} or murine Aurora-A^{T208E}). To validate that the inhibitor indeed targets Aurora-A, (murine) Aurora-A^{wt} and (murine) Aurora-A^{T208D} were overexpressed in a murine neuroblastoma cell line (NHO2A). The kinase resulting from this mutant allele retains catalytic activity but is insensitive to the Aurora-A inhibitor LY3295668. It was shown that overexpressing AURKA^{T208D} rescues proliferation and cell cycle effects in neuroblastoma cells treated with the LY3295668 AURKA inhibitor, which unequivocally demonstrates that LY3295668-dependent inhibition of cell proliferation is an on-target activity of the compound (FIG. 10).

To test the influence of Aurora-A kinase inhibitors on T-cells, proliferation assays were established using CFSE labeling. In first in vitro assays, it was indeed seen that the treatment of T cells with Aurora-A inhibitor impacts the proliferation and activation capacity of T cells (FIG. 11). Since the overexpression of AURKA^{T208D} provides a survival benefit of the tumor cells upon Aurora-A inhibition, resistent (CAR-)Tcells expressing AURKA^{T208D} or the human Aurora-A kinase mutant ^{T217D} can now be established.

Further, the efficacy of hB7H3 CARs on eliminating tumor cells overexpressing hB7H3 has been tested in cocultivation assay (FIG. 12a). For this purpose, the allele AURKA^{T208D} was included in a CAR construct targeting human B7H3 (see FIG. 12b).

Further, infection efficacy is improved and, as next steps, cocultivation and proliferation assays with the CARs expressing AURKA^{T208D} are performed comparing the performance upon treatment with Aurora-A inhibitor and demonstrating the benefit of the resistant CAR T-cells.

### Example 9

Treatment of human tumor cell lines and organoids with CGs leads to a significant reduction in translation of the proto-oncogene MYC, a marked growth disadvantage of treated cells, and downregulation of several immune evasion mechanisms (cf. FIG. 13, left and middle).

An immunoblot of tumor cells after 24 h treatment with cymarin was made (FIG. 14A). Quantification of MYC protein levels in human pancreatic tumor cells and colorectal tumor cells was performed (FIG. 14B). It was shown that CGs (cymarin) reduce the amount of MYC protein in human but not in murine tumor cells (FIG. 14).

Further, it was shown that the depletion of the ATP1A1 subunit of the NA/K pump leads to the reduction of MYC protein levels (FIG. 15A). Ectopic expression of murine ATP1A1 was shown to render the expression of MYC insensitive from the addition of cymarin, a prototypical KG (FIG. 15B). In summary, the effect of CG on MYC protein levels was shown to be mediated by the inhibition of the NA/K pump (FIG. 15).

Further, growth of human colon (DLD1, Ls174T) and pancreatic (PaTu 8988T) and murine pancreatic (KPC) tumor cells under control conditions (DMSO; left) and after treatment with cymarin (100 nM; right) was shown (FIG. 16).

Further, the extracellular acidification rate (lactate secretion) in human LS174 colo-rectal tumor cells was shown to be significantly reduced by treatment with cymarin (FIG. 17). This effect can be reduced by overexpression of the murine 1 isoform of Na⁺/K⁺-ATPase (FIG. 17).

By replacing the endogenous murine ATPase with a human ATPase, MYC expression and lactate secretion in murine pancreatic cancer cells can be inhibited by CGs (FIG. 19A). Such humanized PDAC tumors are completely eradicated by treatment with CGs after transplantation into mice (FIG. 19B). Thus, CGs represent a potential therapeutic agent for cancers/tumors. However, from the known function of Na⁺/K⁺-ATPase and CGs, it is expected that also human immune cells are sensitive to growth inhibition by CGs and that therefore CGs alone will have weak/no effect on immune cell-mediated tumor therapies.

Further, the Na⁺/K⁺-ATPase of human immune cells (e.g. CAR-T-cells) is altered (e.g., by ectopic expression of murine Na⁺/K⁺-ATPase or by CRISPR/Cas9 mutation of endogenous Na⁺/K⁺-ATPase) so that they are resistant to CGs.

Patients with solid tumors are systemically treated with CGs at relevant doses and transfused in parallel with engineered immune cells (e.g. CAR-T-cells) that are resistant to treatment with CGs. This leads to a decrease in MYC protein and immune evasion mechanisms in the tumor with preserved functionality and expansion potential of the modified immune cells which can then eradicate the tumor.

Reference is further made herein to the following table:

TABLE 1
Examples of tumors/cancers and respective markers (derived on
Jul. 20, 2021 from https://en.wikipedia.org/wiki/Tumor_marker).

| Tumor marker | Associated tumor types |
|---|---|
| Alpha | germ cell tumor, hepatocellular carcinoma |
| fetoprotein (AFP) | |
| CA15-3 | breast cancer |
| CA27.29 | breast cancer |
| CA19-9 | Mainly pancreatic cancer, but also colorectal cancer and other types of gastrointestinal |
| | cancer. |
| CA-125 | Mainly ovarian cancer, but may also be elevated in for example endometrial |
| | cancer, fallopian tube cancer, lung cancer, breast cancer and gastrointestinal cancer. May |
| | also increase in Endometriosis. |
| Calcitonin | medullary thyroid carcinoma |
| Calretinin | mesothelioma, sex cord-gonadal stromal tumour, adrenocortical carcinoma, synovial |
| | sarcoma |
| Carcinoembryonic | gastrointestinal cancer, cervix cancer, lung cancer, ovarian cancer, breast cancer, urinary |
| antigen | tract cancer |
| CD34 | hemangiopericytoma/solitary fibrous tumor, pleomorphic lipoma, gastrointestinal |
| | stromal tumor, dermatofibrosarcoma protuberans |
| CD99MIC 2 | Ewing sarcoma, primitive neuroectodermal tumor, hemangiopericytoma/solitary fibrous |
| | tumor, synovial sarcoma, lymphoma, leukemia, sex cord-gonadal stromal tumour |
| CD117 | gastrointestinal stromal tumor, mastocytosis, seminoma |
| Chromogranin | neuroendocrine tumor |
| Chromosomes 3, 7, | bladder cancer |
| 17, and 9p21 | |
| Cytokeratin (various | Many types of carcinoma, some types of sarcoma |
| types: TPA, TPS, | |
| Cyfra21-1) | |
| Desmin | smooth muscle sarcoma, skeletal muscle sarcoma, endometrial stromal sarcoma |
| Epithelial | many types of carcinoma, meningioma, some types of sarcoma |
| membrane | |
| antigen (EMA) | |
| Factor | vascular sarcoma |
| VIII, CD31 FL1 | |
| Glial fibrillary acidic | glioma (astrocytoma, ependymoma) |
| protein (GFAP) | |
| Gross cystic | breast cancer, ovarian cancer, salivary gland cancer |
| disease fluid | |
| protein | |
| (GCDFP-15) | |
| hPG80 | breast cancer, ovarian cancer, prostate cancer, kidney cancer, colorectal cancer, liver |
| | cancer |
| HMB-45 | melanoma, PEComa (for example angiomyolipoma), clear cell carcinoma, adrenocortical |
| | carcinoma |
| Human chorionic | gestational trophoblastic disease, germ cell tumor, choriocarcinoma |
| gonadotropin (hCG) | |
| immunoglobulin | lymphoma, leukemia |
| inhibin | sex cord-gonadal stromal tumour, adrenocortical carcinoma, hemangioblastoma |
| keratin (various | carcinoma, some types of sarcoma |
| types) | |
| lymphocyte | lymphoma, leukemia |
| marker (various | |
| types) | |
| MART-1 (Melan-A) | melanoma, steroid-producing tumors (adrenocortical carcinoma, gonadal tumor) |
| Myo D1 | rhabdomyosarcoma, small, round, blue cell tumour |
| muscle-specific | myosarcoma (leiomyosarcoma, rhabdomyosarcoma) |
| actin (MSA) | |
| neurofilament | neuroendocrine tumor, small-cell carcinoma of the lung |
| neuron-specific | neuroendocrine tumor, small-cell carcinoma of the lung, breast cancer |
| enolase (NSE) | |
| placental alkaline | seminoma, dysgerminoma, embryonal carcinoma |
| phosphatase (PLAP) | |
| prostate-specific | prostate |
| antigen (PSA) | |
| PTPRC (CD45) | lymphoma, leukemia, histiocytic sarcoma |
| S100 protein | melanoma, sarcoma (neurosarcoma, lipoma, chondrosarcoma), astrocytoma, gastrointes |
| | tinal stromal tumor, salivary gland cancer, some types of adenocarcinoma, histiocytic |
| | tumor (dendritic cell, macrophage) |
| smooth muscle | gastrointestinal stromal tumor, leiomyosarcoma, PEComa |
| actin (SMA) | |
| synaptophysin | neuroendocrine tumor |
| thymidine kinase | lymphoma, leukemia, lung cancer, prostate cancer |
| thyroglobulin (Tg) | post-operative marker of thyroid cancer (but not in medullary thyroid cancer) |
| thyroid | all types of thyroid cancer, lung cancer |
| transcription | |
| factor-1 (TTF-1) | |
| Tumor M2-PK | colorectal cancer, Breast cancer, renal cell carcinoma lung cancer, pancreatic |
| | cancer, esophageal cancer, stomach cancer, cervical cancer, ovarian cancer, |
| vimentin | sarcoma, renal cel carcinoma, endometrial cancer, lung |
| | carcinoma, lymphoma, leukemia, melanoma |

1. An immune cell, or a progenitor cell thereof, which is resistant against a DNA damage-inducing agent (DDIA) or which exhibits reduced susceptibility to a DDIA.

2. The immune cell according to claim 1, which is a T-cell or a natural killer (NK) cell, or the progenitor cell according to claim 1 which is a hemocytoblast ((omni- or multipotent) hematopoietic stem cell), a common lymphoid progenitor, a common myeloid progenitor, a lymphoblast or a myeloblast.

3. The immune cell according to claim 1, which expresses a recombinant T-cell receptor and/or an artificial T-cell receptor.

4. The immune cell according to claim 1, which expresses a chimeric antigen receptor (CAR).

5. The immune cell according to claim 3, wherein said receptor specifically binds to a tumor antigen.

6. The immune cell according to claim 3, wherein said receptor specifically binds to a tumor-specific antigen (TSA) or to a tumor-associated antigen (TAA).

7. The immune cell according to claim 1, wherein said immune cell is a CAR T-cell.

8. The immune cell or progenitor cell thereof according to claim 1, which is made resistant against said DDIA by (genetical) engineering or has reduced susceptibility to said DDIA due to (genetical) engineering.

9. The immune cell or progenitor cell thereof according to claim 1, which comprises at least one target of said DDIA which is resistant against said DDIA or has reduced susceptibility to said DDIA.

10. The immune cell or progenitor cell thereof according to claim 9, wherein said target carries a mutation, or two or more mutations, which renders/render said target as being resistant against said DDIA or as having reduced susceptibility to said DDIA.

11. The immune cell or progenitor cell thereof according to claim 9, which comprises at least one allele of said target, wherein said allele carries a mutation, or two or more mutations, which renders/render said target resistant against said DDIA or as having reduced susceptibility to said DDIA.

12. The immune cell or progenitor cell thereof according to claim 1, wherein said resistance or reduced susceptibility against said DDIA is a conditional resistance and reduced susceptibility, respectively (e.g. a resistance and reduced susceptibility, respectively, which is conditional to a FKB analogue, to auxin or an auxin derivative or to a steroid hormone).

13. The immune cell or progenitor cell thereof according to claim 12, wherein said resistance or reduced susceptibility is conditional to doxycycline.

14. The immune cell or progenitor cell thereof according to claim 13, wherein said target of said DDIA is conditionally expressed in the presence of doxycycline.

15. A pharmaceutical composition comprising an immune cell and/or a progenitor cell thereof according to claim 1.

16. A pharmaceutical composition, a kit or a combination (set of two/three components) comprising (e.g. in two/three different vials)
(i) an immune cell and/or a progenitor cell thereof according to claim 1; and
(ii) said DDIA.

17. A method of treating a cancer and/or a tumor in a patient, the method comprising administering to the patient the pharmaceutical composition, kit or combination according to claim 16.

18. The method according to claim 17, wherein said tumor is a malignant and/or metastasizing tumor.

19. The method according to claim 17, wherein said treating comprises the treating of metastases and/or the prevention (of the growth) of metastases.

20. The method according to claim 17, wherein said tumor is a solid tumor.

21. The method according to claim 17, wherein said cancer and/or tumor is a Myc-driven cancer and/or tumor (e.g. a c-Myc-, L-Myc- and/or N-Myc-driven cancer and/or tumor).

22. The method according to claim 17, wherein said cancer and/or tumor is
(i) a pancreas cancer/carcinoma and/or tumor, in particular pancreatic ductal adenocarcinoma (PDAC);
(ii) a colon cancer/carcinoma and/or tumor, in particular metastatic colorectal carcinoma (CRC); or
(iii) (pediatric) neuroblastoma.

23. The method according to claim 17, wherein said treating comprises the treating of metastases and/or the prevention (of the growth) of metastases in the liver.

24. The pharmaceutical composition, kit or combination according to claim 16, wherein said DDIA is a transcription-replication conflict-inducing agent (TRCIA) (this is envisaged to include agents which prevent/target resolution of TRCs).

25. The pharmaceutical composition, kit or combination according to claim 16, wherein said DDIA targets Myc and/or results in a reduction/depletion of (the expression of) Myc.

26. The pharmaceutical composition, kit or combination according to claim 16, wherein the target of said DDIA is a target selected from the group consisting of
(i) Aurora A kinase;
(ii) Na⁺/K⁺-ATPase, in particular human Na⁺/K⁺-ATPase;
(iii) Ataxia telangiectasia and Rad3-related (ATR) kinase;
(iv) PAFc complexes;
(v) PAF1 (e.g. CDC73, LEO1, CTR9);
(vi) CDK9;
(vii) CDK12; CDK13
(viii) cyclinK/CDK12 complexes;
(ix) splicing factors (e.g. SF3B1, RBM39) and/or transcription termination factors; e.g. SPT5, EXOsome
(x) SNRNP70;
(xi) CPSF1; CPSF2
(xii) CPSF3;
(xiii) PNUTs/PPI1 phosphatase complex;
(xiv) NUAK1/ARK5;
(xv) RNA polymerase 1 (POL1);
(xvi) ATM kinase;
(xvii) USP28;
(xviii) Topoisomerase I;
(xix) Topoisomerase II; and
(xx) Poly(ADP-ribose)-Polymerase.

27. The pharmaceutical composition, kit or combination according to claim 16, wherein said DDIA is selected from the group consisting of
(i) an Aurora A kinase inhibitor (e.g. LY3295668, MLN8054, MLN8237 (Alisertib; Millennium));
(ii) a (human) Na⁺/K⁺-ATPase inhibitor (e.g. coumarin, ouabain, digitoxin, cymarin, digoxin, acetyldigitoxin, deslanoside);
(iii) an ATR kinase inhibitor (e.g. AZD6738 (Astra-Zeneca), BAY 1895344 (Bayer));
(iv) a PAFc complex inhibitor or an inhibitor of any subunit of the PAFc complex;
(v) a CDK9 inhibitor (e.g. AZD4573, NVP-2, CYC065 (fadraciclib), THAL-SNS-03);
(vi) a CDK12 inhibitor (e.g. SR4835, THZ-531);
(vii) a cyclinK/CDK12 complexes inhibitor (e.g. CR-8);
(viii) a splicing and/or termination complexes inhibitor (e.g. insidulam, SPI-21 (Bahat, Mol Cell 76, 2019, 617-31 e614), Pladienolide B, H3B-8800)
(ix) a SNRNP70 inhibitor;
(x) a CPSF1 inhibitor;
(xi) a CPSF3 inhibitor (e.g. JTE-607);
(xii) a PNUTs/PPI1 phosphatase complex inhibitor (e.g. calyculin A);
(xiii) a NUAK1/ARK5 inhibitor (e.g. BAY-880 (Bayer), ON-123300, XMD-1571, HTH-01-015);
(xiv) a POL1 inhibitor (e.g. CX-5461);
(xv) ATM kinase inhibitor (e.g. KU-60019, KU-559403, AZD1390);
(xvi) a USP28 inhibitor (e.g. FT206, AZ1);
(xvii) Topoisornerase I inhibitor (e.g. Irinotecan, topotecan, campthotecin);
(xviii) Topoisomerase II inhibitor (e.g. etoposide, doxorubicin, daunorubicin); and
(xix) Poly(ADP-ribose)-Polymerase inhibitor (e.g. olaparib, veliparib).

28. The method according to claim 17, wherein at least two different DDIAs are to be administered.

29. The method according to claim 28, wherein one of said two different DDIAs is (a low dose of) an ATR kinase inhibitor (preferred) or (a low dose of) an ATM kinase inhibitor.

30. The immune cell or progenitor cell thereof according to claim 9, wherein said at least one target of said DDIA which is resistant against said DDIA or has reduced susceptibility to said DDIA and said DDIA, respectively, are selected from the group consisting of
(i) Aurora A kinase T217E or T217D mutant (or another DDIA-resistant Aurora A kinase mutant) and or LY3295668, MLN8054 or MLN8237, respectively (e.g. for use in the treatment of (pediatric) neuroblastoma);
(ii) murine Na⁺/K⁺-ATPase or another Na⁺/K⁺-ATPase with a glutamine (R) at a position which is homolog to position 118 of the murine or human Na⁺/K⁺-ATPase (SEQ ID NOs.2 and 1, respectively) and/or with an asparagine (D) at a position which is homolog to position 129 of the murine or human Na⁺/K⁺-ATPase (SEQ ID NOs.2 and 1, respectively) and coumarin, oubain, digitoxin, cymarin, digoxin, acetyldigitoxin, deslanoside, or another (human) Na⁺/K⁺-ATPase inhibitor (e.g. another CG), respectively (e.g. for use in the treatment of MYC-dependent cancers/tumors, like colon and pancreatic cancers/tumors);
(iii) CDK12 C1039S mutant and THZ-531, respectively (e.g. for use in the treatment of CDK12-dependent tumors, like triple-negative breast cancer/tumor);
(iv) RBM39 G268V mutant and indisulam, respectively (e.g. for use in the treatment of MYC or MYCN-driven tumors, like colon, pancreatic and small cell lung cancers/tumors);
(v) topoisomerase I with (a) mutation(s) that confer(s) resistance to (a) topoisomerase I inhibitor(s) and a topoisomerase I inhibitor, respectively, e.g. a topoisomerase I F361S, G363C and/or R364H mutant and campthotecin, respectively; or a topoisomerase I S365G, R621H and/or E710G mutant and irinotecan, respectively (e.g. for use in the treatment of . . . cancers/tumors);
(vi) topoisomerase II with (a) mutation(s) that confer(s) resistance to (a) topoisomerase inhibitor(s) and a topoisomerase II inhibitor, respectively, e.g. a topoisomerase II P501, G776 and/or K505 mutant and etoposide, doxorubicin or mitoxantron, respectively; and
(vii) a deletion of the cellular PARP gene and a PARP inhibitor, respectively (e.g. olaparib or veliparib) (e.g. for use in the treatment of pediatric tumors).

31. A method of controlling an immune cell therapy, the method comprising administering to a patient an immune cell or progenitor cell thereof according to claim 12 and said DDIA.

32-44. (canceled)


SEQUENCE LISTING (2 sequences)

SEQ ID NO: 1 - protein, 1023 aa, Homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 2 - protein, 1023 aa, Mus musculus
MGKGVGRDKYEPAAVSEHGDKKGKKAKKERDMDELKKEVSMDDHKLSLDELHRKYGTDLSRGLTPARAAEILARDGPNALTPPPTTPEWVKFCRQLFGGFSMLLWIGAILCFLAYGIRSATEEEPPNDDLYLGVVLSAVVIITGCFSYYQEAKSSKIMESFKNMVPQQALVIRNGEKMSINAEDVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAEEIEHFIHLITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWFALSRIAGLCNRAVFQANQENLPILKRAVAGDASESALLKCIEVCCGSVMEMREKYSKIVEIPFNSTNKYQLSIHKNPNASEPKHLLVMKGAPERILDRCSSILLHGKEQPLDEELKDAFQNAYLELGGLGERVLGFCHLLLPDEQFPEGFQFDTDDVNFPVDNLCFVGLISMIDPPRAAVPDAVGKCRSAGIKVIMVTGDHPITAKAIAKGVGIISEGNETVEDIAARLNIPVNQVNPRDAKACVVHGSDLKDMTSEELDDILRYHTEIVFARTSPQQKLIIVEGCQRQGAIVAVTGDGVNDSPALKKADIGVAMGIVGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPFHLLGIRETWDDRWVNDVEDSYGQQWTYEQRKIVEFTCHTAFFVSIVVVQWADLVICKTRRNSVFQQGMKNKILIFGLFEETALAAFLSYCPGMGAALRMYPLKPTWWFCAFPYSLLIFVYDEVRKLIIRRRPGGWVEKETYY